(19) United States
(12) Patent Application Publication
(10) Pub. No.: US 2010/0286215 A1
(43) Pub. Date: Nov. 11, 2010

(54) BIS-AROMATIC COMPOUNDS USEFUL IN THE TREATMENT OF INFLAMMATION

(75) Inventors: Benjamin Pelcman, Uppsala (SE); Peter Nilsson, Uppsala (SE); Martins Katkevics, Riga (LV)

Correspondence Address: K&L Gates LLP, 1900 MAIN STREET, SUITE 600, IRVINE, CA, 92614-7319, US

(73) Assignee: BIS-AROMATIC COMPOUNDS USEFUL IN THE TREATMENT OF INFLAMMATION

(21) Appl. No.: 12/676,575

(22) PCT Filed: Sep. 3, 2008
(86) PCT No.: PCT/GB08/02964

§ 371 Date: May 11, 2010

Related U.S. Application Data
(60) Provisional application No. 60/935,849, filed on Sep. 4, 2007.

(51) Int. Cl. A61K 31/44; C07C 229/00; A61K 31/195; C07C 233/00; C07D 213/78; C07D 333/68; A61K 31/381; C07D 257/04; A61K 31/41; A61P 11/00; A61P 29/00

(52) U.S. Cl. 514/355; 560/19; 514/561; 560/27; 514/563; 546/315; 549/57; 514/443; 560/13; 514/562; 548/250; 514/381

(57) ABSTRACT

There is provided compounds of formula (I): wherein Y, ring A, D₁, D₂, D₃, L¹, Y¹, L², Y², L³ and Y³ have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

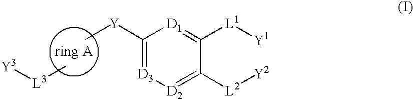

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRas) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRas have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of a prior-published document in this specification should not necessarily be taken as an acknowledgement that the document is part of the state of the art or is common general knowledge.

Various biaryl compounds, which are linked together with a carbonyl group, have been disclosed in journal articles by Antonov et al., Vysokomolekulyarnye Soedineniya (a Russian journal article), Seriya A (1990), 32(2), 310-315; Bogachev et al., ibid (1987), 29(11), 2333-9; Varma at al, Angewandte Makromolekulare Chemie (1988), 157, 59-78; Inou Hiroshi et al., Kagaku to Kogyo (2002), 76(3), 135-140; Sen at al, Journal of Polymer Chemistry, Vol. 34, 25-31 (1996) 25; and Douglas E. Fjare, Macromolecules (1993), 26, 5143-5148. Such compounds have also been disclosed in U.S. Pat. No. 4,892,578 and Russian Patents SU 749859 and SU 78-2620201. However, none of these documents disclose that these compounds have a medical use ascribed to them.

US patent application US 2005/0014169 and international patent application WO 2004/076640 both disclose various biaryl compounds that may act as nuclease inhibitors, with the latter document further stating that the compounds disclosed therein may be useful in the treatment of cancer. However, there in no mention in either document that the compounds disclosed therein may be useful in the treatment of inflammation.

International patent application WO 2006/125593 and European patent application EP 1 113 000 both disclose compounds that may have potential use in the treatment of inflammation. However, the former document predominantly relates to biaryl ring systems that are not further substituted with aromatic groups, and the latter predominantly relates to biaryl compounds that do not contain a carboxylic acid group, or isostere thereof.

International patent applications WO 2006/104957, WO 2006/055625, WO 2005/042520 and WO 01/023347 as well as US patent applications US 2005/0277640 and US 2007/0066660 all disclose various biaryl compounds in which the biaryl group is linked with a carbonyl group (so forming, for example, a benzophenone structure). However, none of these documents mention that the compounds disclosed therein may be useful as inhibitors of LTC₄ synthase, and therefore of use in the treatment of inflammation.

Unpublished PCT application PCT/GB2008/00072 discloses various biphenyl compounds that may be useful in the treatment of inflammation. However, the two phenyl rings are linked together with via a methylene group.

There is no disclosure in any of the prior art documents of biaryl compounds that are linked together with a carbonyl group, in which there is a carboxylic acid (or isostere thereof) and an aryl substituent (attached via a linker group or directly) on one of the aromatic rings of the biaryl system, and an aryl substituent (also attached via a linker group or directly) on the other aromatic ring, for use as LTC₄ synthase inhibitors, and therefore for use in the treatment of inflammation or respiratory disorders.

### DISCLOSURE OF THE INVENTION

According to the invention, there is provided a compound of formula I,

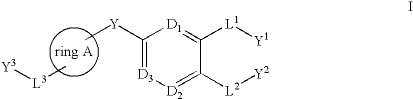

wherein
Y represents —C(O)— or —C(═N—OR²⁸)—;
R²⁸ represents hydrogen or C₁₋₆ alkyl optionally substituted by one or more halo atoms;
each of D₁, D₂ and D₃ respectively represent —C(R^{1a})═, —C(R^{1b})═ and —C(R^{1c})═, or,
each of D₁, D₂ and D₃ may alternatively and independently represent —N═;
ring A represents:

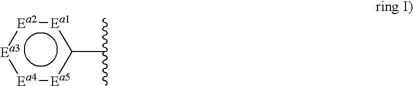

each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{a1}, E^{a2}, E^{a3}, E^{a4} and E^{a5} may alternatively and independently represent —N═;
one of R^{2b}, R^{2c} and R^{2d} represents the requisite -L³-Y³ group, and the others independently represent hydrogen, -L^{1a}-Y^{1a} or a substituent selected from X¹;

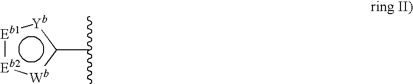

E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or

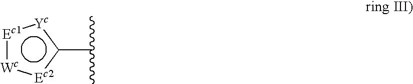

E^{c1} and E^{c2} each respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c}, independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and/or R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and/or —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} (and/or —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and/or —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, and R^{15b} independently represent H, —CH₃ or —CH₂CH₃,
Y¹ and Y^{1a} independently represent, on each occasion when used herein, —N(H)SO₂R^{9a}, —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹ⁱ or any one of the following groups:

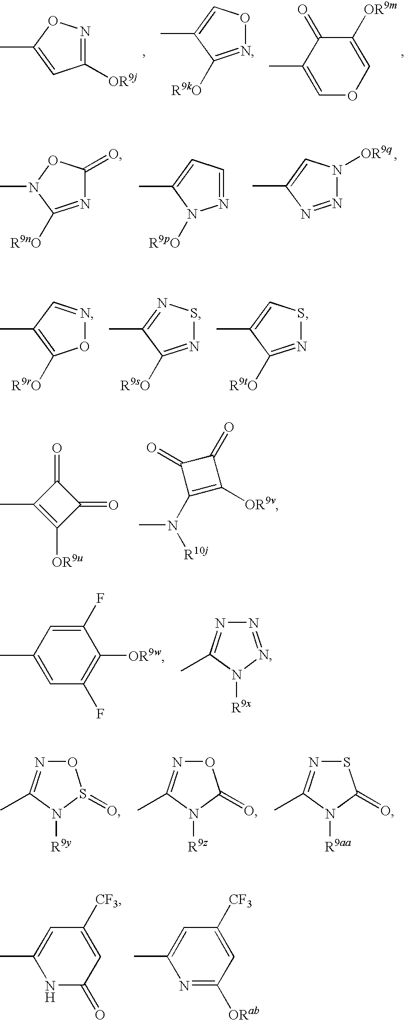

R^{9a} to R^{9z}, R^{9aa}, R^{9ab}, R^{10f}, R^{10g}, R¹⁰ⁱ and R^{10j} independently represent, on each occasion when used herein, C₁₋₈ alkyl or a heterocycloalkyl group, both of which are optionally substituted by one or more substituents selected from G¹ and/or Z¹; or
R^{9b} to R^{9z}, R^{9aa}, R^{9ab}, R^{10f}, R^{10g}, R⁹ⁱ and R¹⁰ⁱ, independently represent, on each occasion when used herein, hydrogen; or
any pair of R^{9f} and R^{10f}, R^{9g} and R^{10g}, and R⁹ⁱ and R¹⁰ⁱ, may be linked together to form, along with the atom(s) to which they are attached, a 3- to 6-membered ring, which ring optionally contains a further heteroatom (such as nitrogen or oxygen), in addition to the nitrogen atom to which these substituents are necessarily attached, and which ring is optionally substituted by one or more substituents selected from F, Cl, ═O, —OR^{5h} and R^{5a};
one of Y² and Y³ represents an aryl group or a heteroaryl group (both of which groups are optionally substituted by one or more substituents selected from A) and the other represents either:
(a) an aryl group or a heteroaryl group (both of which groups are optionally substituted by one or more substituents selected from A); or
(b) C₁₋₁₂ alkyl optionally substituted by one or more substituents selected from G¹ and/or Z¹;

A represents, on each occasion when used herein:

I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)₂A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)₂A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents, on each occasion when used herein, ═O, ═S, ═NOR^{18b}, ═NS(O)₂N(R^{19f})R^{18c}, ═NCN or ═C(H)NO₂;
R^{16a}; R^{16b}; R^{16c}; R^{17a}; R^{17b}; R^{17c}; R^{17d}; R^{17e}; R^{17f}; R^{18a}; R^{18b}; R^{18c}; R^{19a}; R^{19b}, R^{19c};
R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹-R^{20a};
wherein A¹¹ represents a single bond or a spacer group selected from —C(O)A¹²-, —S—, —S(O)₂A¹³-, —N(R^{21a})A¹⁴, or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═NOR^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ and L^{1a} independently represent a single bond or —(CH₂)ₚ-Q-(CH₂)_{q}—;
Q represents —C(R^{y1})(R^{y2})—, —C(O)— or —O—;
R^{y1} and R^{y2} independently represent H, F or X⁴; or
R^{y1} and R^{y2} may be linked together to form a 3- to 6-membered ring, which ring optionally contains a heteroatom, and which ring is optionally substituted by one or more substituents selected from F, Cl, ═O and X⁵;
L² and L³ independently represent a single bond or a spacer group selected from —(CH₂)ₚ—C(R^{y3})(R^{y4})—(CH₂)_{q}-A¹⁶-, —C(O)A¹⁷-, —S—, —SC(R^{y3})(R^{y4})—, —S(O)₂A¹⁸-, —N(R^{w})A¹⁹- or —OA²⁰-, in which:
A¹⁶ represents a single bond, —O—, —N(R^{w})—, —C(O)—, or —S(O)ₘ—;
A¹⁷ and A¹⁸ independently represent a single bond, —C(R^{y3})(R^{y4})—, —O—, or —N(R^{w});
A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;
p and q independently represent, on each occasion when used herein, 0, 1 or 2;
m represents 0, 1 or 2;
R^{y3} and R^{y4} independently represent, on each occasion when used herein, H, F or X⁶; or
R^{y3} and R^{y4} may be linked together to form a 3- to 6-membered ring, which ring optionally contains a heteroatom, and which ring is optionally substituted by one or more substituents selected from F, Cl, ═O and X⁷;
R^{w} represents, on each occasion when used herein, H or X⁸;

X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, —CN, C₁₋₄ alkyl (optionally substituted by one or more substituents selected from fluoro, chloro, and ═O), —N(R^{24c})R^{25b} and —OR^{24d})), aryl or heteroaryl (which latter two groups are optionally substituted by one or more substituents selected from halo, —CN, C₁₋₄ alkyl (optionally substituted by one or more substituents selected from fluoro, chloro and ═O), —N(R^{26a})R^{26b} and —OR^{26c});

R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt thereof,
provided that:
when D₁, D₂ and D₃ all represent —C(H)═; ring A represents ring (I); E^{a1}, E^{a2}, E^{a3}, E^{a4} and E^{a5} respectively represent —C(H)═, —C(R^{2b})═, —C(R^{2c})═, —C(R^{2d})═ and —C(H)═; R^{2d} represents H; L¹ and L^{1a} both represent single bonds; Y¹ and Y^{1a} both represent —C(O)OR^{9b}; R^{9b} represents H:
(A) R^{2c} represents -L³-Y³; R^{2b} represents -L^{1a}-Y^{1a}; L² and L³ both represent —N(R^{w})A¹⁹-; R^{w} represents H; A¹⁹ represents —C(O)—, then Y² and Y³ do not both represent 1-naphthyl;
(B) L² and L³ both represent —C(O)A¹⁷-, A¹⁷ represents —N(R^{w})—; R^{w} represents
(i) R^{2b} represents -L³-Y³; R^{2c} represents -L^{1a}-Y^{1a}, then:
   (I) Y² and Y³ do not both represent 4-pyridyl, 2-pyridyl, 4-methylphenyl or 4-methoxyphenyl;
   (II) Y² and Y³ do not both represent phenyl substituted in the meta-position by a G¹ substituent in which G¹ is chloro, and in the para-position by methyl substituted by G¹, in which G¹ represents -A¹-R^{16a}; A¹ represents a single bond, and R^{16a} represents a heterocycloalkyl group that is 2-isoxazolidinyl group substituted in the 3-position with a Z³ group that is ═O and at the 4-position with two G³ groups in which G³ represents -A¹¹-R^{20a}, A¹¹ is a single bond; and R^{20a} represents —CH₃;
(ii) R^{2c} represents -L³-Y³; R^{2b} represents -L^{1a}-Y^{1a}, then:
   (I) Y² and Y³ do not both represent 4-bromophenyl, phenyl, 4-methylphenyl, 4-methoxyphenyl, 3-nitro-4-aminophenyl or 3-nitro-4-hydroxy-phenyl, or, one of Y² or Y³ does not represent 4-bromophenyl when the other represents unsubstituted phenyl;
   (II) when Y² and Y³ both represent phenyl substituted by A:
      (1) A represents G¹; G¹ represents -A¹-R^{16a}: R^{16a} represents phenyl substituted by G³; G³ represents -A¹¹-R^{20a}; -A¹¹ represents —N(R^{21a})A¹⁴; A¹⁴ represents —C(O)—; R^{21a} represents H; and R^{20a} represents an alkyl group terminally substituted at the same carbon atom with both a ═O and a —OR^{22b} group, in which R^{22b} is hydrogen when:
      (a) A and G³ are both in the para-position, and R^{20a} represents either a C₄ alkyl group that is —CH═C(CH₃)₂ or a C₃ alkyl group that is —C(H)═C(H)—CH₃ (both of which are terminally substituted at one of the CH₃ groups), then when A¹ represents —OA⁵-, then A⁵ does not represent a single bond;
      (b) A and G³ are both in the para-position, and R^{20a} represents —CH═C(CH₃)₂ (terminally substituted at one of the CH₃ groups), then when A¹ represents —S(O)₂A³, then A³ does not represent a single bond;
      (c) A and G³ are both in the meta-position, and R^{20a} represents a —C(H)═C(H)—CH₃ (terminally substituted at the CH₃ group), then when A¹ represents —S(O)₂A³, then A³ does not represent a single bond;
      (2) A represents methyl substituted by G¹; G¹ represents -A¹-R^{16a}, A¹ represents a single bond, R^{16a} phenyl substituted in the para-position by G³; G³ represents -A¹¹-R^{20a}; -A¹¹ presents —N(R^{21a})A¹⁴; A¹⁴ represents —C(O)—; R^{21a} represents H; and R^{20a} represents either a C₄ alkyl group that is —CH₂—C(═CH₂)—CH₃ or a C₃ alkyl group that is —C(H)═C(H)—CH₃, then the latter two alkyl groups are not both terminally substituted at the respective —CH₃ moieties with both a ═O and a —OR^{22b} group, in which R^{22b} is hydrogen,
         which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

The term “halo”, when used herein, includes fluoro, chloro, bromo and iodo. Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl) and, more preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazopyridyl (such as imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiazolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which X¹ and X² both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{3a} and —C(O)R^{3b} in which R^{3b} represents R^{3a}, then the identities of the two R^{3a} groups are not to be regarded as being interdependent. Likewise, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{5a} to R^{5h}” is employed herein, this will be understood by the skilled person to mean R^{4a}, R^{4b}, R^{4c}, R^{4d}, R^{4e}, R^{4f}, R^{4g} and R^{4h} inclusively.

For the avoidance of doubt, when the term “an R⁵ group” is referred to herein, we mean any one of R^{5a} to R^{5k}, R^{5m}, R⁵ⁿ or R^{5p}.

For the avoidance of doubt, where it is stated herein that “any pair of R^{16a} to R^{16c} and R^{17a} to R^{17f} . . . may . . . be linked together”, we mean that any one of R^{16a}, R^{16b} or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—) or R^{16c} and R^{17f} may be linked together with the nitrogen atom to which they are necessarily attached to form a ring as hereinbefore defined.

The skilled person will appreciate that, given that there is an essential ‘-L³-Y³’ group present in the compound of formula I, then when, for example, ring A represents ring I), then at least one of —C(R^{2b})═, —C(R^{2c})═ and —C(R^{2d})═ must be present, in which the any one of the relevant R^{2b}, R^{2c} and R^{2d} groups represents the essential -L³-Y³ group.

Compounds of the invention that may be mentioned include those in which:

Y¹ and Y^{1a} independently represent, on each occasion when used herein, —N(H)SO₂R^{9a}, —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹ⁱ or any one of the following groups:

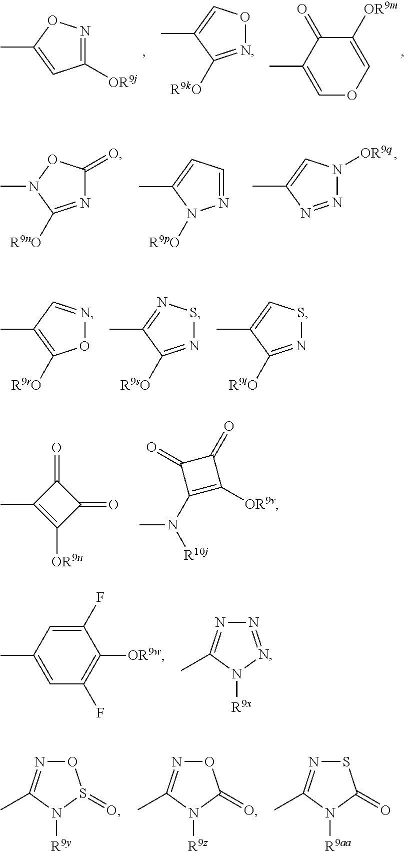

M¹ and M² independently represent —CH₂CH₃, or, preferably, —CH₃, —CF₃ or —N(R^{15a})R^{15b};
R^{11a} and R^{13a} independently represent —CHF₂ or, preferably H, —CH₃, —CH₂CH₃ or —CF₃;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d})) aryl or heteroaryl (which latter two groups are optionally substituted by one or more substituents selected from halo, C₁₋₄ alkyl (optionally substituted by one or more substituents selected from fluoro, chloro and ═O), —N(R^{26a})R^{26b} and —OR^{26c});
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from chloro or, preferably, fluoro and/or ═O.

Further compounds of the invention that may be mentioned include those in which:

Y² and Y³ independently represent an aryl group or a heteroaryl group, both of which groups are optionally substituted by one or more substituents selected from A;
Y represents —C(O)—.

Further compounds of the invention that may be mentioned include those in which:

one of Y² and Y³ represents an aryl group or a heteroaryl group (both of which groups are optionally substituted by one or more substituents selected from A) and the other represents C₁₋₁₂ alkyl optionally substituted by one or more substituents selected from G¹ and/or Z¹; and/or
Y represents —C(═N—OR²⁸).

Compounds of the invention that may be mentioned include those in which, for example, when D₁, D₂ and D₃ respectively represent —C(R^{1a})═, —C(R^{1b})═ and —C(R^{1c})═; ring A represents ring (I) and Eel, E^{a1}, E^{a3}, E^{a4} and E^{a5} respectively represent —C(H)═, —C(R^{2b})═, —C(R^{2b})═, —C(R^{2d})═ and —C(H)═, then:

when Y² and Y³ both represent a heteroaryl (e.g. a 4- to 10-membered heteroaryl) group, then L¹ and, if present, L^{1a}, independently represent a single bond, —(CH₂)ₚ-Q-(CH₂)_{q}— in which Q represents —C(O)—, or, —(CH₂)ₚ-Q-(CH₂)_{q}— in which p represents 1 or 2 and Q represents —O—;
when R^{3a} represents C₁₋₆ alkyl substituted with two substituents, then those substituents are not ═O and —OR^{8a} substituted at a terminal carbon atom of the alkyl group (so forming a —C(═O)OR^{3a} group);
when Y² and Y³ both represent a heteroaryl group, then L² and L³ do not both represent single bonds.

Further compounds of the invention that may be mentioned include those in which, for example, when D₁, D₂ and D₃ respectively represent —C(R^{1a})═, —C(R^{1b})═ and —C(R^{1c})═; ring A represents ring (I) and E^{a1}, E^{a2}, E^{a3}, E^{a4} and E^{a5} respectively represent —C(H)═, —C(R^{2b})═, —C(R^{2c})═, —C(R^{2d})═ and —C(H)═, then:

L¹ represents a single bond, —(CH₂)ₚ-Q-(CH₂)_{q}— in which Q represents —C(O)—, or, —(CH₂)ₚ-Q-(CH₂)_{q}— in which p represents 1 or 2 and Q represents —O—;
Q represents —C(O)—;
R^{5a} represents, on each occasion when used herein, C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} or —OS(O)₂N(R^{8g})R^{8h};
R^{5a} represents, on each occasion when used herein, C₁₋₆ alkyl optionally substituted by one or more substituents selected from halo, —CN, —N₃, ═O, —N(R^{8b})R^{8c}, —S(O)ₙR^{8d}, —S(O)₂N(R^{8e})R^{8f} or —OS(O)₂N(R^{8g})R^{8h};
(e.g. one of) L² and L³ independently represent a spacer group selected from —(CH₂)ₚ—C(R^{y3})(R^{y4})—(CH₂)_{q}-A¹⁶-, —C(O)A¹⁷-, —S—, —SC(R^{y3})(R^{y4})—, —S(O)₂A¹⁸-, —N(R^{w})A¹⁹- or —OA²⁰-;
(e.g. one of) Y² and Y³ represent an aryl group optionally substituted as defined herein.

Further compounds of the invention that may be mentioned include those in which, for example, when D₁, D₂ and D₃ respectively represent —C(H)═, —C(R^{1b})═ and —C(H)═; ring A represents ring (I) and E^{a1}, E^{a2}, E^{a3}, E^{a4} and E^{a5} respectively represent —C(H)═, —C(R^{2b})═, —C(R^{2c})═, —C(R^{2d})═ and —C(H)═, when R^{1b} or, if present, X¹ represent —N(R^{5d})C(O)R^{6c}, and R^{6c} represents R^{5a}, then R^{5a} represents a linear or branched C₁₋₆ alkyl group optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} or —OS(O)₂N(R^{8g})R^{8h}.

Yet further compounds of the invention that may be mentioned include those in which:

when, for example, ring A represents ring (I), L² or L³ represent —N(R^{w})A¹⁹-, in which A¹⁹ represents a single bond and R^{w} represents H, then Y² or Y³ (as appropriate) do not represent a benzimidazolyl (e.g. benzimidazol-2-yl) group.

Preferred compounds of the invention include those in which:

one (e.g. D₁ or D₃) or none of D₁, D₂ and D₃ represent —N═;
D₁, D₂ and D₃ respectively represent —C(R^{1a})═, —C(R^{1b})═ and —C(R^{1c})═;
R^{1a} and R^{1c} independently represent hydrogen;
when ring A represents ring (I), then two, preferably, one or, more preferably, none of E^{a1}, E^{a2}, E^{a3}, E^{a4} and E^{a5} represent —N═;
E^{a1}, E^{a2}, E^{a3}; E^{a4} and E^{a5} respectively represent —C(H)═, —C(R^{2b})═, —C(R^{2c})═, —C(R^{2d})═ and —C(H)═;
R^{2c} represents the requisite -L³-Y³ group;
only one of R^{2b}, R^{2c} and R^{2d} (e.g. R^{2b}) may represent -L^{1a}-Y^{1a};
one of R^{2b} and R^{2d} (e.g. R^{2b}) represents hydrogen or -L^{1a}-Y^{1a}, and the other represents hydrogen or a substituent selected from X¹;
when one of R^{2b}, R^{2c} and R^{2d} represents -L^{1a}-Y^{1a}, then it is preferably tetrazolyl or, more preferably, —COOR^{9b}, in which R^{9b} is preferably H;
R^{3c} and R^{3d} independently represent unsubstituted C₁₋₆ (e.g. C₁₋₃) alkyl, or, preferably, hydrogen;
for example when ring A represents ring (II) then, one of R^{3a} and R^{3b} represents a substituent X² or, more preferably, H or -L^{1a}-Y^{1a}, and the other represents the requisite -L³-Y³ group;
R^{4b} and R^{4c} independently represent unsubstituted (e.g. C₁₋₃) alkyl, or, preferably, hydrogen;
for example when ring A represents ring (III) then, one of R^{4a} and, if present, R^{4d} represents a substituent X³ or, more preferably, H or -L^{1a}-Y^{1a}, and the other represents the requisite -L³-Y³ group;
when any one of R^{3a}, R^{3b}, R^{3c}, R^{3d}, R^{4a}, R^{4b}; R^{4c} or R^{4d} (e.g. R^{3a}; R^{3b}; R^{4a} or R^{4d}) represents -L^{1a}-Y^{1a}, then it is preferably a 5-tetrazolyl group or —COOR^{9b}, in which
R^{9b} is preferably H;
X¹, X² and X³ independently represent halo (e.g. chloro or fluoro), —R^{5a}, —CN, —NO₂ and —OR^{5h};
Z^{1a} and Z^{2a} independently represent —R^{5a};
when any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ are linked together, they form a 5- or 6-membered ring optionally substituted by F, —OCH₃ or, preferably, ═O or R^{5a}, and which ring optionally contains an oxygen or nitrogen heteroatom (which nitrogen heteroatom may be optionally substituted, for example with a methyl group, so forming e.g. —N(H)— or —N(CH₃)—);
R^{5c}, R^{5j} and R^{6e} independently represent R^{5a};
when R^{5a}, R^{8a}, R^{8b}, R^{8d}, R^{8e} and represent C₁₋₆ alkyl optionally substituted by one or more halo substituents, then those halo substituents are preferably Cl or, more preferably, F;
R^{5a} represents C₁₋₆ (e.g. C₁₋₄) alkyl optionally substituted by one or more substituents selected from Cl, —N₃, ═O, —N(R^{8b})R^{8c} and, preferably, F and —OR^{8a};
m and n independently represent 2;
when any one of R^{8a}, R^{8b}, R^{8d}, R^{8e} and R^{8g} represents C₁₋₆ alkyl substituted by halo, then preferred halo groups are chloro and, preferably, fluoro;
R^{8a}, R^{8b}, R^{8d}, R^{8e} and R^{8g} independently represent H or C₁₋₃ alkyl optionally substituted by one or more fluoro atoms;
R^{8c}, R^{8f} and R^{8h} independently represent H, —S(O)₂CH₃, —S(O)₂CF₃ or C₁₋₃ alkyl optionally substituted by one or more fluoro atoms, or the relevant pairs (i.e. R^{8b} and R^{8c}, R^{8e} and R^{8f} or R^{8g} and R^{8h}) are linked together as defined herein;
when R^{8b} and R^{8c}, R^{8e} and R^{8f} or R^{8g} and R^{8h} are linked together, they form a 5- or 6-membered ring, optionally substituted by F, ═O or —CH₃;
M¹ and M² independently represent —CH₃ or —CF₃;
R^{11a}, R^{12a}, R^{12b}, R^{13a}, R^{14a}, R^{14b}, R^{15a} and R^{15b} independently represent H or —CH₃;
Y¹ and Y^{1a} independently represent —C(O)OR^{9b}, —S(O)₂N(R¹⁰ⁱ)R⁹ⁱ or 5-tetrazolyl;
when Y¹ and/or Y^{1a} represents —P(O)(OR^{9d})₂, then, preferably, one R^{9d} group represents hydrogen and the other represents an alkyl group as defined herein (so forming a —P(O)(O-alkyl)(OH) group);
when any pair of R^{9f} and R^{10f}, R^{9g} and R^{10g}, R⁹ⁱ and R¹⁰ⁱ are linked together to form a 3- to 6-membered ring as hereinbefore defined, that ring is optionally substituted by one or more substituents selected from Cl or, preferably, F, ═O and/or R^{5a};
R^{9a} represents C₁₋₄ (e.g. C₁₋₃) alkyl optionally substituted by one or more halo (e.g. fluoro) atoms;
R^{9b} to R^{9z}, R^{9 aa}, R^{9ab}, R^{10f}, R^{10g}, R¹⁰ⁱ and R^{10j} independently represent hydrogen or C₁₋₄ (e.g. C₁₋₃) alkyl optionally substituted by one or more halo (e.g. fluoro) atoms;
R^{9b} represents H;
R¹⁰ⁱ represents H;
R⁹ⁱ represents hydrogen or C₁₋₃ alkyl (such as methyl, ethyl and isopropyl);
A represents aryl (e.g. phenyl) optionally substituted by B; C₁₋₆ alkyl optionally substituted by G¹ and/or Z¹; or G¹;
G¹ represents halo, cyano, N₃, —NO₂ or -A¹-R^{16a};
A¹ represents —C(O)A², —N(R^{17a})A⁴- or —OA⁵-;
A² represents a single bond or —O—;
A⁴ represents —C(O)N(R^{17d})—, —C(O)O— or, more preferably, a single bond or —C(O)—;
A⁵ represents —C(O)— or, preferably, a single bond;
Z¹ represents ═NCN, preferably, ═NOR^{16b} or, more preferably, ═O;
B represents heteroaryl (e.g. oxazolyl, thiazolyl, thienyl or, preferably, pyridyl) or, more preferably, aryl (e.g. phenyl) optionally substituted by G²; C₁₋₆ alkyl optionally substituted by G² and/or Z²; or, preferably G²,
G² represents cyano or, more preferably, halo, —NO₂ or -A⁶-R^{18a};
A⁶ represents a single bond, —N(R^{19a})A⁹- or —OA¹⁰-;
A⁹ represents —C(O)N(R^{19d})—, —C(O)O— or, more preferably, a single bond or —C(O)—;
A¹⁰ represents a single bond;
Z² represents ═NCN, preferably, ═NOR^{18b} or, more preferably, ═O;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from hydrogen, aryl (e.g. phenyl) or heteroaryl (which latter two groups are optionally substituted by G³) or C₁₋₆ (e.g. C₁₋₄) alkyl (optionally substituted by G³ and/or Z³), or the relevant pairs are linked together as hereinbefore defined;
when any pair of R^{16a} to R^{16c} and R^{17a} to R^{17f}, or R^{18a} to R^{18c} and R^{19a} to R^{19f} are linked together, they form a 5- or 6-membered ring, optionally substituted by one or more (e.g. one or two) substituents selected from G³ and/or Z³;
G³ represents halo or -A¹¹-R^{20a};
A¹¹ represents a single bond or —O—;
Z³ represents ═O;
R^{20a}, R^{20b}, R^{20c}, R^{21a}, R^{21b}, R^{21c}, R^{21d}, R^{21e} and R^{21f} are independently selected from H, C₁₋₃ (e.g. C₁₋₂) alkyl (e.g. methyl) optionally substituted by one or more halo (e.g. fluoro) atoms, or optionally substituted aryl (e.g. phenyl), or the relevant pairs are linked together as defined herein;
when any pair of R^{20a} to R^{20c} and R^{21a} to R^{21f} are linked together, they form a 5- or 6-membered ring, optionally substituted by one or more (e.g. one or two) substituents selected from halo (e.g. fluoro) and C₁₋₂ alkyl (e.g. methyl);
R^{y1} and R^{y2} independently represent hydrogen or methyl, or, they are linked together to form a 3-membered cyclopropyl group;
either one of p and q represents 1 and the other represents 0, or, more preferably, both of p and q represent 0;
Q represents —C(R^{y1})(R^{y2})— or —C(O)—;
L² and L³ independently represent —OA²⁰-, particularly, —S—, —SC(R^{y3})(R^{y4})— or, preferably, —(CH₂)ₚ—C(R^{y3})(R^{y4})—(CH₂)_{q}-A¹⁸-, —S(O)₂A¹⁸- or —N(R^{w})A¹⁹-;
A¹⁸ represents a single bond or, preferably, —C(O)—;
A¹⁹ represents —N(R^{w})— or, preferably, a single bond;
A¹⁹ represents —C(R^{y3})(R^{y4})—, —C(O)O—, —C(O)C(R^{y3})(R^{y4})— or, preferably, a single bond, —C(O)—, —C(O)N(R^{w})— or —S(O)₂—;
A²⁰ represents a single bond or —C(R^{y3})(R^{y4})—;
R^{y3} and R^{y4} independently represent H or X⁶, or, are linked together to form a 3-membered cyclopropyl group;
R^{w} represents H or X⁸;
X⁴ to X⁸ independently represent C₁₋₃ alkyl (optionally substituted by fluoro) or aryl (e.g. phenyl) optionally substituted by fluoro;
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} independently represent hydrogen or C₁₋₂ alkyl optionally substituted by ═O or, more preferably, one or more fluoro atoms.

More preferred compounds of the invention include those in which:

when ring A represents ring (I), in which there is one —N═ group present, then E^{a1}, E^{a3} or E^{a5} represents such a substituent;
when ring A represents ring (II), then W^{b} may represent —N(R^{3d})— (so forming a pyrrolyl or imidazolyl ring) or, more preferably, when Y^{b} represents —C(R^{3c})═, then W^{b} preferably represents —O— or, particularly, —S-(so forming a furanyl or, particularly, a thienyl ring) or when Y^{b} represents —N═, then W^{b} preferably represents —O— or —S-(so forming, for example, an oxazolyl or thiazolyl ring);
R^{3c} and R^{3d} independently represent H;
when ring A represents ring (III), then W^{c} preferably represents —N(R^{4d})—;
R^{4d} represents H;
R^{8c}, R^{8f} and R^{8h} independently represent H or C₁₋₃ alkyl optionally substituted by one or more fluoro atoms;
X¹, X² and X³ independently represent fluoro, chloro, —CN, methyl, ethyl, isopropyl, difluoromethyl, trifluoromethyl, —NO₂, methoxy, ethoxy, difluoromethoxy and/or trifluoromethoxy;
R^{y1} and R^{y2} independently represent hydrogen;
A represents G¹ or C₁₋₆ alkyl (e.g. C₁₋₄ alkyl) optionally substituted by G¹ and/or Z¹;
A¹ represents —N(R^{17a})A⁴- or —OA⁵-;
G² represents halo or -A⁶-R^{18a}.

Preferred rings that ring A may represents include furanyl (e.g. 2-furanyl), thienyl (e.g. 2-thienyl), oxazolyl (e.g. 2-oxazolyl), thiazolyl (e.g. 2-thiazolyl), pyridyl (e.g. 2- or 4-pyridyl), pyrrolyl (e.g. 3-pyrrolyl), imidazolyl (e.g. 4-imidazolyl) or, preferably, phenyl.

Preferred rings that the D₁ to D₃-containing ring may represent include 2- or 4-pyridyl (relative to the point of attachment to the —C(O)— moiety) or, preferably, phenyl.

Preferred aryl and heteroaryl groups that Y² and Y³ may independently represent include optionally substituted (i.e. by A) phenyl, naphthyl (e.g. 5,6,7,8-tetrahydronaphthyl), pyrrolyl, furanyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include benzothienyl (e.g. 7-benzothienyl), 1,3-benzodioxolyl, particularly, naphthyl (e.g. 5,6,7,8-tetrahydronaphthyl or, preferably, 1-naphthyl or 2-naphthyl), more particularly, 2-benzoxazolyl, 2-benzimidazolyl, 2-benzothiazolyl, thienyl, oxazolyl, thiazolyl, pyridyl (e.g. 2- or 3-pyridyl), and, most preferably, phenyl.

Preferred substituents on Y² and Y³ groups include:

halo (e.g. fluoro, chloro or bromo);
cyano;

### —NO₂;

C₁₋₆ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²⁶;

### —C(O)R²⁶;

### —C(O)OR²⁶; and

### —N(R²⁶)R²⁷;

wherein R²⁶ and R²⁷ independently represent, on each occasion when used herein, H, C₁₋₆ alkyl, such as C₁₋₄ alkyl (e.g. ethyl, n-propyl, t-butyl or, preferably, n-butyl, methyl or isopropyl) optionally substituted by one or more halo (e.g. fluoro) groups (so forming e.g. a perfluoroethyl or, preferably, a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms).

Preferred compounds of the invention include those in which:

D₁ and D₃ independently represent —C(H)═;
D₂ represents —C(R^{1b})═;
R^{1b} represents H;
ring A represents ring (I);
E^{a1} and E^{a5} independently represent —C(H)═;
E^{a2}, E^{a3}, and E^{a4} respectively represent —C(R^{2b})═, —C(R^{2c})═ and —C(R^{2d})═;
R^{2b} represents H or -L^{1a}-Y^{1a};
R^{2c} represents the requisite -L³-Y³ group;
R^{2d} represents H;
L¹ and L^{1a} independently represent a single bond;
L¹ and L^{1a} are the same;
Y¹ and Y^{1a} independently represent 5-tetrazolyl (which is preferably unsubstituted) or, preferably, —C(O)OR^{9b};
Y¹ and Y^{1a} are the same;
when Y¹ represents 5-tetrazolyl, then R^{2b} to R^{2d} (e.g. R^{2b}) do not represent -L^{1a}-Y^{1a} (but preferably represent hydrogen);
R^{9b} represents C₁₋₆ alkyl (e.g. ethyl or methyl) or H;
when, for example, Y¹ and Y^{1a} are the same, then R^{9b} represents C₁₋₆ alkyl (e.g. ethyl or, preferably, methyl) or, more preferably, H;
L² and L³ independently represent —OA²⁰- or, preferably, —N(R^{w})A¹⁹-;
at least one of L² and L³ represents —N(R^{w})A¹⁹-;
L² and L³ may be different (for example when R^{2b} represents H) or L² and L³ are the same (for example when R^{2b} represents -L^{1a}-Y^{1a});
A¹⁹ represents a single bond, —S(O)₂—, —C(O)— or —C(O)N(R^{w})—;
A²⁰ represents a single bond;
R^{w} represents C₁₋₃ alkyl (e.g. methyl) or H;
Y² and Y³ independently represent heteroaryl (such as 6-membered monocyclic heteroaryl group in which the heteroatom is preferably nitrogen or a 9-membered bicyclic heteroaryl group in which there is one or two heteroatom(s) preferably selected from sulfur and oxygen; so forming a pyridyl group, e.g. 2-pyridyl or 3-pyridyl, benzothienyl, e.g. 7-benzothienyl, or benzodioxoyl, e.g. 4-benzo[1,3]dioxoyl) or, preferably, aryl (e.g. naphthyl, such as 5,6,7,8-tetrahydronaphthyl, or, preferably, phenyl) both of which are optionally substituted by one or more (e.g. one or two) substituents selected from A; at least one of Y² and Y³ represents aryl (e.g. phenyl) optionally substituted as defined herein;
Y² and Y³ may be different (for example when R^{2b} represents H) or Y² and Y³ are the same (for example when R^{2b} represents -L^{1a}-Y^{1a});
when Y² or Y³ represent C₁₋₁₂ alkyl, then it is preferably a C₁₋₆ alkyl group (e.g. an unsubstituted acyclic C₁₋₆ alkyl group, a part-cyclic C₁₋₆ alkyl group, such as cyclopentylmethyl, or, a cyclic C₃₋₆ alkyl group, such as cyclohexyl), optionally substituted by one or more G¹ substituent(s), in which G¹ is preferably -A¹-R^{16a}; A¹ is a single bond and R^{16a} is a (preferably unsubstituted) C₁₋₆ (e.g. C₁₋₄) alkyl group (e.g. tert-butyl);

A represents G¹ or C₁₋₆ (e.g. C₁₋₄) alkyl (e.g. butyl (such as n-butyl) or methyl) optionally substituted by one or more substituents selected from G¹;

G¹ represents halo (e.g. chloro or fluoro), NO₂ or -A¹-R^{16a};
A¹ represents a single bond or, preferably, —OA⁵-;
A⁵ represents a single bond;
R^{16a} represents hydrogen or C₁₋₆ (e.g. C₁₋₄) alkyl optionally substituted by one or more substituents selected from G³ (e.g. R^{16a} may represent ethyl or, preferably, butyl (such as tert-butyl or, preferably n-butyl), propyl (such as isopropyl) or methyl);
G³ represents halo (e.g. fluoro; and hence e.g. R^{16a} may represent trifluoromethyl or perfluoroethyl);
when Y² and/or Y³ represent an optionally substituted phenyl group, then that phenyl group may be substituted with a single substituent (e.g. at the para- (or 4-) position) or with two substituents (e.g. with one at the para-position and the other at the meta- or ortho- (3- or 2-) position, so forming for example a 3,4-substituted, 2,4-substituted or 2,5-substituted phenyl group);
R²⁸ represents hydrogen or unsubstituted C₁₋₃ (e.g. C₁₋₂) alkyl (e.g. methyl).

Preferred substituents on Y² or Y³ groups (for instance, when they represent heteroaryl groups or, preferably, aryl group, such as phenyl) include 1,1,2,2-tetrafluoroethoxy, 2,2,2-trifluoroethoxy, preferably ethoxy, methoxy and, more preferably, halo (e.g. chloro and fluoro), —NO₂, trifluoromethyl, butyl (e.g. n-butyl), trifluoromethoxy, isopropoxy, n-butoxy and hydroxy.

When Y² or Y³ represents optionally substituted C₁₋₁₂ alkyl, then that group is preferably cyclohexyl (e.g. (4-tert-butyl)cyclohexyl), hexyl (e.g. n-hexyl) or cyclopentylmethyl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which Y represents —C(O)—, oxidation of a compound of formula II,

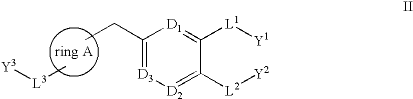

wherein ring A, D₁, D₂, D₃, L¹, Y¹, L², Y², L³ and Y³ are as hereinbefore defined, in the presence of a suitable oxidising agent, for example, KMnO₄, optionally in the presence of a suitable solvent, such as acetone, and an additive such as magnesium sulfate;
(ii) for compounds of formula I in which L² and/or L³ represents —N(R^{w})A¹⁹- in which R^{w} represents H (and, preferably, Y is —C(O)— and/or R²⁸ is C₁₋₆ alkyl optionally substituted by one or more halo atoms), reaction of a compound of formula III,

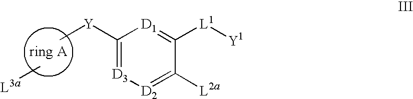

or a protected derivative thereof (e.g. an amino-protected derivative or a keto-protecting group, such as a ketal or thioketal) wherein L^{2a} represents —NH₂ or —N(R^{w})A¹⁹-Y², L^{3a} represents —NH₂ or —N(R^{w})A¹⁹-Y³, provided that at least one of L^{2a} and L^{3a} represents —NH₂, and Y, ring A, D₁, D₂, D₃, L¹ and Y¹ are as hereinbefore defined, with:
(A) when A¹⁹ represents —C(O)N(R^{w})—, in which R^{w} represents H:
   (a) a compound of formula IV,

Y^{a}—N═C═O IV
; or
(b) with CO (or a reagent that is a suitable source of CO (e.g. Mo(CO)₆ or Co₂(CO)₈)) or a reagent such as phosgene or triphosgene in the presence of a compound of formula V,

Y^{a}—NH₂ V

wherein, in both cases, Y^{a} represents Y² or Y³ (as appropriate/required) as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). The skilled person will appreciate that for preparation of compounds of formula I in which -L²-Y² represents —C(O)N(H)—Y² and -L³-Y³ represents —C(O)N(H)—Y³ and Y² and Y³ are different, two different compounds of formula IV or V (as appropriate) will need to be employed in successive reaction steps. For the preparation of such compounds starting from compounds of formula III in which both of L^{2a} and L^{3a} represent —NH₂, then mono-protection (at a single amino group) followed by deprotection may be necessary, or the reaction may be performed with less than 2 equivalents of the compound of formula IV or V (as appropriate);
(B) when A¹⁹ represents —S(O)₂N(R^{w})—:
   (a) ClSO₃H, followed by PCl₅, and then reaction with a compound of formula V as hereinbefore defined;
   (b) SO₂Cl₂, followed by reaction with a compound of formula V as hereinbefore defined;
   (c) a compound of formula VA,

Y^{a}—N(H)SO₂Cl VA
wherein Y^{a}Y^{a} is as hereinbefore defined;
(d) ClSO₂N═C═O, optionally in the presence BrCH₂CH₂OH, following by reaction in the presence of a compound of formula V as hereinbefore defined (which reaction may proceed via a 2-oxazolidinone intermediate),
   for example under standard reaction conditions, for e.g. such as those described hereinbefore in respect of process step (ii)(A) above (e.g. employing a Cu or Pd catalyst under Goldberg coupling or Buchwald-Hartwig reaction conditions), followed by standard oxidation reaction conditions (for example, reaction in the presence of an oxidising reagent such as meta-chloroperbenzoic acid in the presence of a suitable solvent such as dichloromethane e.g. as described in Journal of Organic Chemistry, (1988) 53(13), 3012-16, or, KMnO₄, e.g. as described in Journal of Organic Chemistry, (1979), 44(13), 2055-61. The skilled person will also appreciate that the compound of formula VA may need to be prepared, for example from a corresponding compound of formula V as defined above, and SO₂ (or a suitable source thereof) or SOCl₂;
   (C) when A¹⁹ represents a single bond, with a compound of formula VI,

Y^{a}-L^{a} VI

wherein L^{a} represents a suitable leaving group such as chloro, bromo, iodo, a sulfonate group (e.g. —OS(O)₂CF₃, —OS(O)₂OH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y^{a} is as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation;
(D) when A¹⁹ represents —S(O)₂—, —C(O)—, —C(R^{y3})(R^{y4})—, —C(O)—C(R^{y3})(R^{y4})— or —C(O)O—, with a compound of formula VII,

Y^{a}-A^{19a}-L^{a} VII

wherein A^{19a} represents —S(O)₂—, —C(O)—, —C(R^{y3})(R^{y4})—, —C(O)—C(R^{y3})(R^{y4})— or —C(O)O—, and r and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(iii) for compounds of formula I in which one of L² and L³ represents —N(R^{w})C(O)N(R^{w})— and the other represents —NH₂ (or a protected derivative thereof) or —N(R^{w})C(O)N(R^{w})—, in which R^{w} represents H (in all cases), and, preferably, Y is —C(O)— and/or R²⁸ is C₁₋₆ alkyl optionally substituted by one or more halo atoms, reaction of a compound of formula VIII,

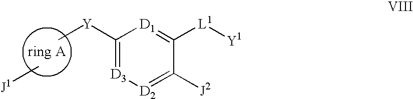

wherein one of J¹ or J² represents —N═C═O and the other represents —NH₂ (or a protected derivative thereof) or —N═C═O (as appropriate), and Y, ring A, D₁, D₂, D₃, L¹ and Y¹ are as hereinbefore defined, with a compound of formula V as hereinbefore defined, under reaction conditions known to those skilled in the art, such as those described hereinbefore in respect of process step (ii)(A)(b) above;
(iv) for compounds of formula I in which, preferably, Y is —C(O)— and/or R²⁸ is C₁₋₆ alkyl optionally substituted by one or more halo atoms, reaction of a compound of formula IX,

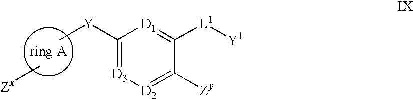

wherein at least one of Z^{x} and Z^{y} represents a suitable leaving group and the other may also independently represent a suitable leaving group, or, Z^{y} may represent -L²-Y² and Zₓ may represent -L³-Y³, in which the suitable leaving group may independently be fluoro or, preferably, chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and Y, ring A, D₁, D₂, D₃, L¹, Y¹, L², Y², L³ and Y³ are as hereinbefore defined, with a (or two separate) compound(s) (as appropriate/required) of formula X,

Y^{a}-L^{x}-H X

wherein Lx represents L² or L³ (as appropriate/required), and Y^{a} is as hereinbefore defined, under suitable reaction conditions known to those skilled in the art, for example such as those hereinbefore described in respect of process (ii)(B) above or (e.g. when L^{x} represents —S(O)₂A¹⁸-, in which A¹⁸ represents —N(R^{w})—) under e.g. Ullman reaction conditions such as those described in Tetrahedron Letters, (2006), 47(28), 4973-4978. The skilled person will appreciate that when compounds of formula I in which L² and L³ are different are required, then reaction with different compounds of formula X (for example, first reaction with a compound of formula X in which Lx represents —N(R^{w})A¹⁹-, followed by reaction with another, separate, compound of formula X in which L^{x} represents —OA²⁰-) may be required;
(v) compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), may be prepared by reaction of a corresponding compound of formula I in which such a group is present that does represent hydrogen with a compound of formula XI,

R^{wy}-L^{b} XI

wherein R^{wy} represents either R^{w} (as appropriate) as hereinbefore defined provided that it does not represent hydrogen (or R^{w} represents a R⁵ to R¹⁹ group in which those groups do not represent hydrogen), and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (ii)(C) above. The skilled person will appreciate that various groups (e.g. primary amino groups) may need to be mono-protected and then subsequently deprotected following reaction with the compound of formula XI;
(vi) for compounds of formula I that contain only saturated alkyl groups, reduction of a corresponding compound of formula I that contains an unsaturation, such as a double or triple bond, in the presence of suitable reducing conditions, for example by catalytic (e.g. employing Pd) hydrogenation;
(vii) for compounds of formula I in which Y¹ and/or, if present, Y^{1a} represents —C(O)OR^{9b}, —S(O)₃R^{9c}, —P(O)(OR^{9d})₂, or—B(OR^{9h})₂, in which R^{9b}, R^{9c}, R^{9d} and R^{9h} represent hydrogen (or, e.g. in the case of compounds in which Y¹ and/or Y^{1a} represent —C(O)OR^{9b}, other carboxylic acid or ester protected derivatives (e.g. amide derivatives)), hydrolysis of a corresponding compound of formula I in which R^{9b}, R^{9c}, R^{9d} or R^{9h} (as appropriate) does not represent H, or, for compounds of formula I in which Y represents —P(O)(OR^{9d})₂ or S(O)₃R^{9c}, in which R^{9c} and R^{9d} represent H, a corresponding compound of formula I in which Y represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate), all under standard conditions, for example in the presence of an aqueous solution of base (e.g. aqueous 2M NaOH) optionally in the presence of an (additional) organic solvent (such as dioxane or diethyl ether), which reaction mixture may be stirred at room or, preferably, elevated temperature (e.g. about 120° C.) for a period of time until hydrolysis is complete (e.g. 5 hours);
(viii) for compounds of formula I in which Y¹ and/or, if present, Y^{1a} represents —C(O)OR^{9b}, S(O)₃R^{9c}, —P(O)(OR^{9d})₂, —P(O)(OR^{9d})₂, —P(O)(OR^{9e})N(R^{10f})R^{9f} or —B(OR^{9h})₂ and R^{9b} to R^{9e} and R^{9h} (i.e. those R⁹ groups attached to an oxygen atom) do not represent H:
(A) esterification (or the like) of a corresponding compound of formula I in which R^{9b} to R^{9e} and R^{9h} represent H; or
(B) trans-esterification (or the like) of a corresponding compound of formula I in which R^{9b} to R^{9e} and R^{9h} do not represent H (and does not represent the same value of the corresponding R^{9b} to R^{9a} and R^{9h} group in the compound of formula Ito be prepared),
   under standard conditions in the presence of the appropriate alcohol of formula XII,

R^{9za}OH XII

in which R^{9za} represents R^{9b} to R^{9e} or R^{9h} (as appropriate) provided that it does not represent H, for example further in the presence of acid (e.g. concentrated H₂SO₄) at elevated temperature, such as at the reflux temperature of the alcohol of formula XII;
(ix) for compounds of formula I in which Y¹ and/or, if present, Y^{1a} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, and R¹⁰ⁱ are other than H, and L¹ and/or, if present, L^{1a}, are as hereinbefore defined, provided that they do not represent —(CH₂)ₚ-Q-(CH₂)_{q}— in which p represents 0 and Q represents —O—, and, preferably, Y is —C(O)— and/or R²⁸ is C₁₋₆ alkyl optionally substituted by one or more halo atoms, reaction of a compound of formula XIII,

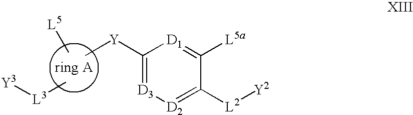

wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (as appropriate), and Y, ring A, D₁, D₂, D₃, L², Y², L³ and Y³ are as hereinbefore defined (the skilled person will appreciate that the compound of formula XIII in which L⁵ and/or L^{5a} represents an alkali metal (e.g. lithium), a Mg-halide or a zinc-based group may be prepared from a corresponding compound of formula XIII in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XIV,

L⁶-L^{xy}-Y^{b} XIV

wherein L^{xy} represents L¹ or L^{1a} (as appropriate) and Y^{b} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})R₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L⁶ represents a suitable leaving group known to those skilled in the art, such as halo (especially chloro or bromo), for example when Y^{b} represents —C(O)OR^{9b} or —S(O)₃R^{9c}, or C₁₋₃ alkoxy, for example when Y^{b} represents —B(OR^{9h})₂. For example, for compounds of formula I in which L¹ represents a single bond and Y¹ represents —C(O)OR^{9b}, the compound of formula XIV may be Cl—C(O)OR^{9b}. The reaction may be performed under standard reaction conditions, for example in the presence of a polar aprotic solvent (e.g. THF or diethyl ether). The skilled person will appreciate that compounds of formula XIII in which L⁵ represents —B(OH)₂ are also compounds of formula I;
(x) compounds of formula I in which L¹ and/or, if present, L^{1a} represent a single bond, and Y¹ and/or, if present, Y^{1a} represent either: —B(OR^{9h})₂ in which R^{9h} represents H; —S(O)₃R^{9c}; or any one of the following groups:

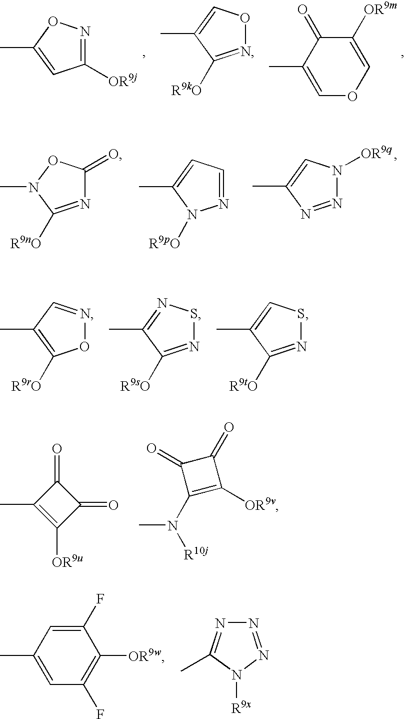

in which R^{9j}, R^{9k}, R^{9m}, R⁹ⁿ, R^{9p}, R^{9r}, R^{9s}, R^{9t}, R^{9u}, R^{9v}, R^{10j} and R^{9x} represent hydrogen, and R^{9w} is as hereinbefore defined (and, preferably, Y is —C(O)— and/or R²⁸ is C₁₋₆ alkyl optionally substituted by one or more halo atoms), may be prepared in accordance with the procedures described in international patent application WO 2006/077366;
(xi) compounds of formula I in which L¹ and/or, if present, L^{1a} represent a single bond, and Y¹ and/or, if present, Y^{1a} represent any one of the following groups:

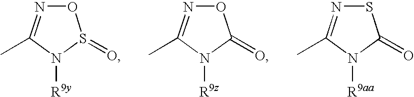

in which R^{9y}, R^{9z} and R^{9aa} represent H, may be prepared by reaction of a compound corresponding to a compound of formula I, but in which Y¹ and/or, if present, Y^{1a} represents —CN, with hydroxylamine (so forming a corresponding hydroxyamidino compound) and then with SOCl₂, R^{j}—OC(O)Cl (e.g. in the presence of heat; wherein R^{j} represents a C₁₋₆ alkyl group) or thiocarbonyl diimidazole (e.g. in the presence of a Lewis Acid such as BF₃.OEt₂), respectively, for example under reaction conditions such as those described in Naganawa et al, Bioorg. Med. Chem., (2006), 14, 7121.
(xii) compounds of formula I in which L¹ and/or, if present, L^{1a} represent a single bond, and Y¹ and/or, if present, Y^{1a} represent any one of the following groups:

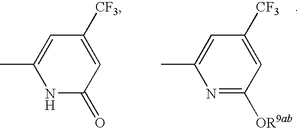

in which R^{9ab} is as hereinbefore defined (and, preferably, Y is —C(O)— and/or R²⁸ is C₁₋₆ alkyl optionally substituted by one or more halo atoms), may be prepared by reaction of a compound of formula XIII wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (as appropriate), and ring A, D₁, D₂, D₃, L², Y², L³ and Y³ are as hereinbefore defined (the skilled person will appreciate that the compound of formula XIII in which L⁵ and/or L^{5a} represents an alkali metal (e.g. lithium), a Mg-halide or a zinc-based group may be prepared from a corresponding compound of formula XIII in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XIVa or XIVb,

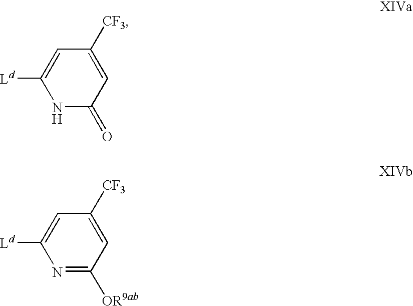

wherein R^{ab} is as hereinbefore defined and L^{d} represents (as appropriate) an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), the skilled person will appreciate that the compound of formula XIVa or XIVb in which L^{d} represents an alkali metal (e.g. lithium), a Mg-halide or a zinc-based group may be prepared from a corresponding compound of formula XIVa or XIVb in which L^{d} represents halo, for example under conditions such as Grignard reaction conditions, halogen-lithium exchange reaction conditions, which latter two may be followed by transmetallation, all of which reaction conditions are known to those skilled in the art. The reaction may be performed under standard reaction conditions, for example in the presence of a suitable solvent (e.g. THF, diethyl ether, dimethyl formamide) and, if appropriate, in the presence of a suitable catalyst (e.g. Pd(OAc)₂) and base (e.g. K₂CO₃). The skilled person will appreciate that compounds of formula XIII in which L⁵ represents —B(OH)₂ are also compounds of formula I;
(xiii) for compounds of formula I in which L¹ and/or, if present, L^{1a} represent a single bond, and Y¹ and/or, if present, Y^{1a} represent —C(O)OR^{9b} in which R^{9b} is H, (and, preferably, Y is —C(O)— and/or R²⁸ is C₁₋₆ alkyl optionally substituted by one or more halo atoms), reaction of a compound of formula XIII as hereinbefore defined but in which L⁵ and/or L^{5a} (as appropriate) represents either:
(I) an alkali metal (for example, such as one defined in respect of process step (ix) above); or
(II) —Mg-halide,
   with carbon dioxide, followed by acidification under standard conditions known to those skilled in the art, for example, in the presence of aqueous hydrochloric acid; (xiv) for compounds of formula I in which L¹ and/or, if present, L^{1a} represent a single bond, and Y¹ and/or, if present, Y^{1a} represent —C(O)OR^{9b} (and, preferably, Y is —C(O)— and/or R²⁸ is C₁₋₆ alkyl optionally substituted by one or more halo atoms), reaction of a corresponding compound of formula XIII as hereinbefore defined but in which L⁵ and/or L^{5a} (as appropriate) is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound of formula XV,

R^{9b}OH XV

wherein R^{9b} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;
(xv) for compounds of formula I in which Y represents —C(O)—, reaction of either a compound of formula XVI or XVII,

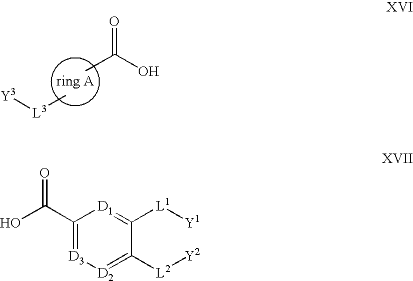

respectively with a compound of formula XVIII or XIX,

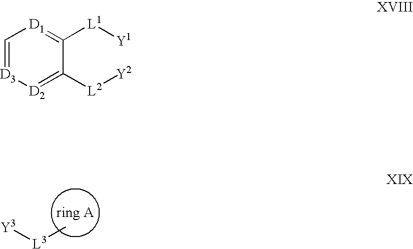

wherein (in all cases) ring A, D₁, D₂, D₃, L¹, Y¹, L², Y², L³ and Y³ are as hereinbefore defined, in the presence of a suitable reagent that converts the carboxylic acid group of the compound of formula XVI or XVII to a more reactive derivative (e.g. an acid chloride or acid anhydride, or the like) such as POCl₃, in the presence of ZnCl₂, for example as described in Organic and Biomolecular Chemistry (2007), 5(3), 494-500 or, more preferably, PCl₅, PCl₅, SOCl₂ or (COCl)₂. Alternatively, such a reaction may be performed in the presence of a suitable catalyst (for example a Lewis acid catalyst such as SnCl₄), for example as described in Journal of Molecular Catalysis A: Chemical (2006), 256(1-2), 242-246 or under alternative Friedel-crafts acylation reaction conditions (or variations thereupon) such as those described in Tetrahedron Letters (2006), 47(34), 6063-6066; Synthesis (2006), (21), 3547-3574; Tetrahedron Letters (2006), 62(50), 11675-11678; Synthesis (2006), (15), 2618-2623; Pharmazie (2006), 61(6), 505-510; and Synthetic Communications (2006), 36(10), 1405-1411. Alternatively, such a reaction between the two relevant compounds may be performed under coupling reaction conditions (e.g. Stille coupling conditions), for example as described in Bioorganic and Medicinal Chemistry Letters (2004), 14(4), 1023-1026;
(xvi) for compounds of formula I in which Y represents —C(O)—, reaction of either a compound of formula XX or XXI,

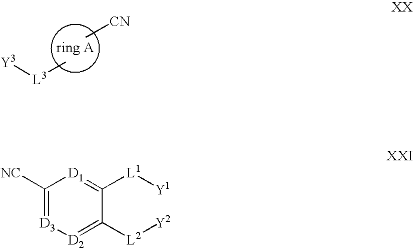

with a compound of formula XXII or XXIII,

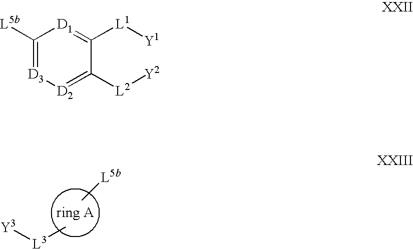

respectively, wherein L^{5b} represents L⁵ as hereinbefore defined provided that it does not represent -L¹-Y¹, and which L^{5b} group may therefore represents —B(OH)₂ (or a protected derivative thereof), an alkali metal (such as lithium) or a —Mg-halide (such as —MgI or, preferably, —MgBr), and (in all cases) ring A, D₁, D₂, D₃, L¹, Y¹, L², Y², L³ and Y³ are as hereinbefore defined, and (in the case of compounds of formulae XXII and XXIII), for example in the presence of a suitable solvent, optionally in the presence of a catalyst, for example, as described in Organic Letters (2006), 8(26), 5987-5990. Compounds of formula I may also be obtained by performing variations of such a reaction, for example by performing a reaction of a compound of formula XX or XXI respectively with a compound of formula XVIII or XIX as hereinbefore defined, for example under conditions described in Journal of Organic Chemistry (2006), 71(9), 3551-3558 or US patent application US 2005/256102;
(xvii) for compounds of formula I in which Y represents —C(O)—, reaction of an activated derivative of a compound of formula XVI or XVII as hereinbefore defined (for example an acid chloride; the preparation of which is hereinbefore described in process step (xv) above), with a compound of formula XXII or XXIII (as hereinbefore defined), respectively, for example under reaction conditions such as those hereinbefore described in respect of process step (xvi) above;
(xviii) for compounds of formula I in which Y represents —C(═N—OR²⁸)—, reaction of a corresponding compound of formula I, with a compound of formula XXIIIA,

H₂N—O—R²⁸ XXIIIA

wherein R²⁸ is represents hydrogen or C₁₋₆ alkyl optionally substituted by one or more halo atoms, under standard condensation reaction conditions, for example in the presence of an anhydrous solvent (e.g. dry pyridine, ethanol and/or another suitable solvent);
(xix) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXIIIB,

R^{28a}-L⁷ XXIIIB

wherein R^{28a} represents R²⁸, provided that it does not represent hydrogen and L⁷ represents a suitable leaving group, such as one hereinbefore defined in respect of L^{a} (e.g. chloro or bromo), under standard alkylation reaction conditions, such as those hereinbefore described in respect of process step (ii);
(xx) compounds of formula I in which -L¹-Y¹ and/or, if present, -L^{1a}-Y^{1a} represent —S(O)₃H, may be prepared by reaction (sulfonylation) of a compound corresponding to a compound of formula I, but in which -L¹-Y¹ and/or -L^{1a}-Y^{1a} (as appropriate) represents hydrogen, with a suitable reagent for the introduction of the sulfonic acid group, such as sulfuric acid at an appropriate concentration (e.g. concentrated, fuming or H₂SO₄*H₂O), SO₃ (i.e. oleum) and/or a halosulfonic acid (e.g. followed by hydrolysis), under conditions known to those skilled in the art;
(xxi) compounds of formula I in which -L¹-Y¹ and/or, if present, -L^{1a}-Y^{1a} represent —S(O)₃H, may be prepared by oxidation of a compound corresponding to a compound of formula I, but in which -L¹-Y¹ and/or -L^{1a}-Y^{1a} (as appropriate) represents —SH, under standard oxidation conditions, for example employing HNO₃ (e.g. boiling nitric acid) or m-chloroperbenzoic acid in, where necessary, an appropriate solvent system (e.g. dichloromethane).

Compounds of formula II may be prepared by reaction of a compound of formula XVIII with a compound of formula XIX, both as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.). Preferably, the formaldehyde is added (e.g. slowly) to an acidic solution of the compound of formula XVIII at about 50° C., with the reaction temperature rising to about 70° C. after addition is complete. When acidic conditions are employed, precipitation of the compound of formula II may be effected by the neutralisation (for example by the addition of a base such as ammonia). Compounds of formula I may also be prepared in accordance with such a procedure, for example under similar reaction conditions, employing similar reagents and reactants.

Compounds of formulae III, VIII, IX and XIII in which Y represents —C(O)—, may be prepared by oxidation of a compound of formulae XXIV, XXV, XXVI and XXVII, respectively,

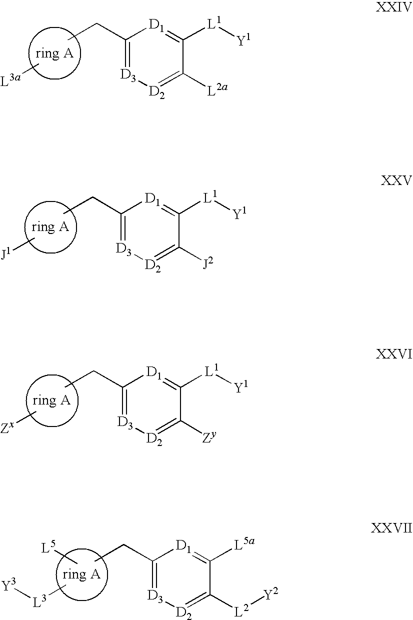

wherein ring A, D₁, D₂, D₃, L¹, Y¹, L^{2a}, L^{3a}, Z^{x}, Z^{y}, L², Y², L³, Y³, J¹, J², L⁵ and L^{5a} are as hereinbefore defined, under standard oxidation conditions known to those skilled in the art, for example such as those hereinbefore described in respect of preparation of compounds of formula I (process step (i) above). The skilled person will appreciate that, similarly, compounds of formulae XXIV, XXV, XXVI and XXVII may be prepared by reduction of corresponding compounds of formulae III, VIII, IX and XIII, under standard reaction conditions, such as those described herein.

Compounds of formula III in which Y represents —C(O)—, or, preferably, XXIV (or protected, e.g. mono-protected derivatives thereof) may be prepared by reduction of a compound of formula XXVIII,

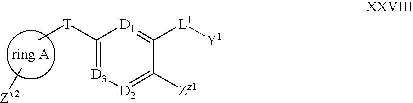

wherein T represents —C(O)— (in the case where compounds of formula III are to be prepared) or, preferably, —CH₂— (in the case where compounds of formula XXIV are to be prepared), Z^{z1} represents —N₃, —NO₂, —N(R^{w})A¹⁹-Y² or a protected —NH₂ group, Z^{z2} represents —N₃, —NO₂, —N(R^{w})A¹⁹-Y³ or a protected —NH₂ group, provided that at least one of Z^{z1} and Z^{z2} represents —N₃ or —NO₂, under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane). The skilled person will appreciate that where the reduction is performed in the presence of a —C(O)— group (e.g. when T represents —C(O)—), a chemoselective reducing agent may need to be employed.

Compounds of formula III in which both L^{2a} and L^{3a} represent —NH₂ (or protected derivatives thereof) may also be prepared by reaction of a compound of formula IX as defined above, with ammonia, or preferably with a protected derivative thereof (e.g. benzylamine or Ph₂C═NH), under conditions such as those described hereinbefore in respect of preparation of compounds of formula I (process step (iv) above).

Compounds of formulae III, IX, XXIV or XXVI in which L¹ represents a single bond, and Y¹ represents —C(O)OR^{9b}, may be prepared by:

(I) reaction of a compound of formula XXIX,

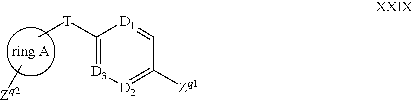

wherein Z^{q1} and Z^{q2} respectively represent Zₓ and Z^{y} (in the case of preparation of compounds of formulae IX or XXVI) or —NH₂ (or —N(R^{w})A¹⁹-Y², —N(Fr)A¹⁹-Y³ or a protected derivative thereof; in the case of preparation of compounds of formulae III or XXIV), and ring A, D₁, D₂, D₃, Z^{x}, Z^{y} and T are as hereinbefore defined, with a suitable reagent such as phosgene or triphosgene in the presence of a Lewis acid, followed by reaction in the presence of a compound of formula XV as hereinbefore defined, hence undergoing a hydrolysis or alcoholysis reaction step;
(II) for such compounds in which R^{9b} represents hydrogen, formylation of a compound of formula XXIX as hereinbefore defined, for example in the presence of suitable reagents such as P(O)Cl₃ and DMF, followed by oxidation under standard conditions;
(III) reaction of a compound of formula XXX,

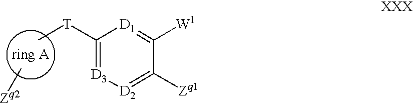

wherein W¹ represents a suitable leaving group such as one defined by Zₓ and Z^{y} above, and ring A, D₁, D₂, D₃, Z^{q1}, Z^{q2} and T are as hereinbefore defined, are as hereinbefore defined, with CO (or a reagent that is a suitable source of CO (e.g. Mo(CO)₆ or CO₂(CO)₈) followed by reaction in the presence of a compound of formula XV as hereinbefore defined, under reaction conditions known to those skilled in the art, for example such as those hereinbefore described in respect of preparation of compounds of formula I (process step (ii)(A)(b) above), e.g. the carbonylation step being performed in the presence of an appropriate precious metal (e.g. palladium), catalyst;
(IV) reaction of a compound of formula XXXI,

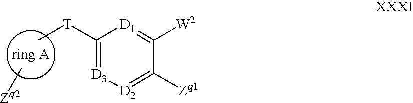

wherein W² represents a suitable group such as an appropriate alkali metal group (e.g. sodium, potassium or, especially, lithium), a —Mg-halide or a zinc-based group, and ring A, Dᵢ, D₂, D₃, Z^{q1}, Z^{q2} and T are as hereinbefore defined, with e.g. CO₂ (in the case where R^{9b} in the compounds to be prepared represents hydrogen) or a compound of formula XIV in which L^{xy} represents a single bond, Y^{b} represents —C(O)OR^{9b}, in which R^{9b} is other than hydrogen, and L⁶ represents a suitable leaving group, such as chloro or bromo or a C₁₋₁₄ (such as C₁₋₈ (e.g. 3) alkoxy group), under reaction conditions known to those skilled in the art. The skilled person will appreciate that this reaction step may be performed directly after (i.e. in the same reaction pot) the preparation of compounds of formula XXXI (which is described hereinafter).

Compounds of formula IX in which Zₓ and Z^{y} represent a sulfonate group may be prepared from corresponding compounds in which the Zₓ and Z^{y} groups represent a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i), e.g. an aqueous solution of K₃PO₄ in toluene) preferably at or below room temperature (e.g. at about 10° C.).

Compounds of formulae XXII and XXIII in which L^{5b} represents a —Mg-halide may be prepared by reaction of a compound corresponding to a compound of formula XXII or XXIII but in which L^{5b} represents a halo group (e.g. bromo or iodo), under standard Grignard formation conditions, for example in the presence of i-PrMgCl (or the like) in the presence of a polar aprotic solvent (such as THF) under inert reaction condition, and preferably at low temperature (such as at below 0° C., e.g. at about 30° C.). The skilled person will appreciate that these compounds may be prepared in situ (see e.g. the process for the preparation of compounds of formula I (process steps (xvi) and (xvii)).

Compounds of formulae XXIX or XXX in which T represents —CH₂— may be prepared by reduction of a corresponding compound of formulae XXIX or XXX in which T represents —C(O)— (or from compounds corresponding to compounds of formulae XXIX or XXX but in which T represents —CH(OH)—), for example under standard reaction conditions known to those skilled in the art, for example reduction in the presence of a suitable reducing reagent such as LiAlH₄, NaBH₄ or trialkylsilane (e.g. triethylsilane) or reduction by hydrogenation (e.g. in the presence of Pd/C).

Alternatively, compounds of formulae XXIX or XXX in which T represents —CH₂— may be prepared by reaction of a compound of formula XXXII,

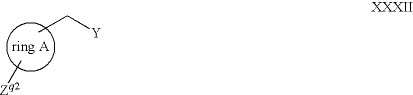

wherein Y represents a suitable group such as —OH, bromo, chloro or iodo, and ring A and Z^{q2} are as hereinbefore defined, with a compound of formula XXXIII,

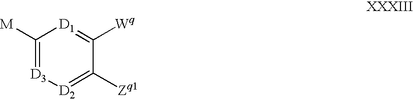

wherein M represents hydrogen and W^{q} represents hydrogen (for compounds of formula XXIX) or W¹ (for compounds of formula XXX) and D₁, D₂, D₃ and Z^{q1} are as hereinbefore defined, under standard conditions, for example in the presence of a Lewis or Brønsted acid. Alternatively, such compounds may be prepared from reaction of a compound of formula XXXII in which Y represents bromo or chloro with a compound corresponding to a compound of formula XXXIII but in which M represents —BF₃K (or the like), for example in accordance with the procedures described in Molander et al, J. Org. Chem. 71, 9198 (2006).

Compounds of formulae XXIX or XXX in which T represents —C(O)— may be prepared by reaction of a compound of formula XXXIV,

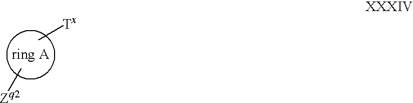

wherein T^{x} represents —C(O)Cl or —C═N—NH(t-butyl) (or the like) and ring A and Z^{q2} are as hereinbefore defined, with a compound of formula XXXIII in which M represents hydrogen or an appropriate alkali metal group (e.g. sodium, potassium or, especially, lithium), a —Mg-halide or a zinc-based group, or, a bromo group, and D₁, D₂, D₃, Z^{q1} and W^{q} are as hereinbefore defined, under reaction conditions known to those skilled in the art. For example in the case of reaction of a compound of formula XXXIV in which T^{x} represents —C(O)Cl with a compound of formula XXXIII in which M represents hydrogen, in the presence of an appropriate

Lewis acid. In the case where M represents an appropriate alkali metal group, a —Mg-halide or a zinc-based group, under reaction conditions such as those hereinbefore described in respect of preparation of compounds of formulae III, IX, XXIV or XXVI (process step (IV) above) and preparation of compounds of formula XXXI (see below). In the case of a reaction of a compound of formula XXXIV in which T^{x} represents —C═N—NH(t-butyl) (or the like) with a compound of formula XXXIII in which M represents bromo, under reaction conditions such as those described in Takemiya et al, J. Am. Chem. Soc. 128, 14800 (2006).

For compounds corresponding to compounds of formula XXIX or XXX in which T represents —CH(OH)—, reaction of a compound corresponding to a compound of formula XXXIV, but in which T^{x} represents —C(O)H, with a compound of formula XXXIII as defined above, under reaction conditions such as those hereinbefore described in respect of preparation of compounds of formulae XXIX or XXX in which T represents —C(O)—.

Compounds of formula XXXI may be prepared in several ways. For example, compounds of formula XXXI in which W² represents an alkali metal such as lithium, may be prepared from a corresponding compound of formula XXIX (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamido group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XXXI may be prepared by reaction of a compound of formula XXX in which W¹ represents chloro, bromo or iodo by a halogen-lithium reaction in the presence of an organolithium base such as t- or n-butyllithium under reaction conditions such as those described above. Compounds of formula XXXI in which W² represents —Mg-halide may be prepared from a corresponding compound of formula XXX in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XXXI in which W² represents a zinc-based group (e.g. using ZnCl₂).

Compounds of formulae IV, V, VA, VI, VII, X, XI, XII, XIII, XIV, XIVa, XIVb, XV, XVI, XVII, XVIII, XIX, XX, XXI, XXII, XXIII, XXIIIA, XXIIIB, XXV, XXVII, XXVIII, XXXII, XXXIII and XXXIV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents D₁, D₂, D₃, L¹, Y¹, L², Y², L³ and Y³ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ (or, if present, Y^{1a}) represents —C(O)OR^{9b} in which R^{9b} does not initially represent hydrogen (so providing at least one ester functional group), the skilled person will appreciate that at any stage during the synthesis (e.g. the final step), the relevant R^{9b}-containing group may be hydrolysed to form a carboxylic acid functional group (i.e. a group in which R^{9b} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include the reduction of a nitro group to an amino group, the hydrolysis of a nitrile group to a carboxylic acid group, and standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or, preferably, potassium cyanide) as a reagent (alternatively, in this case, palladium catalysed cyanation reaction conditions may also be employed).

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate nucleophile e.g. a source of the relevant anions, for example by reaction in the presence of a halogen gas (e.g. bromine, iodine or chlorine), or a reagent that is a source of azido or cyanide anions, such as NaN₃ or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing heterocycles may also be performed with compounds that are pre-cursors to heterocycles, and which pre-cursors may be converted to those heterocycles at a later stage in the synthesis.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an α-carboxy group (i.e. such that the cyclic moiety is formed between the two functional groups.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-interscience (1999).

### Medical and Pharmaceutical Uses

Compounds of the invention are indicated as pharmaceuticals. According to a further aspect of the invention there is provided a compound of the invention, as hereinbefore defined but without proviso (B), for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention, including, but not limited to:
(a) compounds of formula I in which Y¹ (or, if present, Y^{1a}) represents —C(O)OR^{9b} in which R^{9b} is/are other than hydrogen, so forming an ester group; and/or
(b) compounds of formula I in which Y represents —C(═N—OR²⁸)—, i.e. the following compound of formula Ia,

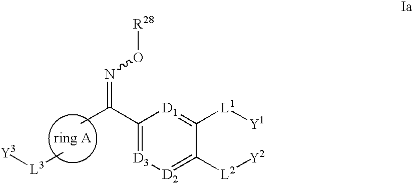

in which the integers are as hereinbefore defined (and the squiggly line indicates that the oxime may exist as a cis or trans isomer, as is apparent to the skilled person),
   may possess no or minimal pharmacological activity as such, but may be administered parenterally or orally, and thereafter be metabolised in the body to form compounds of the invention that possess pharmacological activity as such, including, but not limited to:
(A) corresponding compounds of formula I, in which Y¹ (or, if present, Y^{1a}) represents —C(O)OR^{9b} in which R^{9b} represent hydrogen (see (a) above); and/or
(B) corresponding compounds of formula I in which Y represents —C(O)—, for example in the case where the oxime or oxime ether of the compound of formula Ia (see (b) above) is hydrolysed to the corresponding carbonyl moiety.

Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III (preferably, MGST-II), thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTC₄ and/or LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the an to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, LOffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemic, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without proviso (B), in admixture with a pharmaceutically acceptable adjuvant, diluent or carrier.

The invention further provides a process for the preparation of a pharmaceutical formulation, as hereinbefore defined, which process comprises bringing into association a compound of the invention, as hereinbefore defined but without proviso (B), or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. leukotriene receptor antagonists (LTRas), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activating protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined but without the provisos; and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos, another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined but without the provisos, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined but without the provisos, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another); which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay LTC₄ synthase catalyses the reaction where the substrate LTA₄ methyl ester is converted to LTC₄ methyl ester. Recombinant human LTC₄ synthase is expressed in Piccia pastoralis and the purified enzyme is dissolved in 25 mM Tris-buffer pH 7.8 and stored at −20° C. The assay is performed in phosphate buffered saline (PBS) pH 7.4, supplemented with 5 mM glutathione (GSH). The reaction is terminated by addition of acetonitrile/MeOH/acetic acid (50/50/1). The assay is performed at rt in 96-well plates. Analysis of the formed LTC₄ methyl ester is performed with reversed phase HPLC (Waters 2795 utilizing an Onyx Monolithic C18 column). The mobile phase consists of acetonitrile/MeOH/H₂O (32.5/30/37.5) with 1% acetic acid pH adjusted with NH₃ to pH 5.6, and absorbance measured at 280 nm with a Waters 2487 UV-detector.

The following is added chronologically to each well:
1. 50 μl assay buffer, PBS with 5 mM GSH.
2. 0.5 μl inhibitor in DMSO.
3. 2 μl LTC₄ synthase in PBS. The total protein concentration in this solution is 0.025 mg/ml. Incubation of the plate at room temperature for 10 minutes.
4. 0.5 μl LTA₄ methyl ester. Incubation of the plate at rt for 1 min.
5. 50 μl stop solution. 80 μl of the incubation mixture is analysed with HPLC.

### EXAMPLES

The invention is illustrated by way of the following examples, in which the following abbreviations may be employed:

aq aqueous
atm atmosphere
brine saturated solution of NaCl in water
DMAP N,N-dimethyl-4-aminopyridine
DMF dimethylformamide
DPE-phos 2,2′-bis(diphenylphosphino)diphenyl ether
EtOAc ethyl acetate
MeOH methanol
NMR nuclear magnetic resonance
Pd/C palladium on charcoal
rt room temperature
rx reflux temperature
sat saturated
THF tetrahydrofuran

Chemicals specified in the synthesis of the compounds in the examples were commercially available from, e.g. Sigma-Aldrich Fine Chemicals or Acros Int.

### Examples 1 to 13

### Preparation of Starting Materials and Active Inhibitors

### Dimethyl 3,3′-methylenebis(6-aminobenzoate) I

Methyl 2-amino benzoate (15.1 g, 100 mmol) was added to water (126 mL) at 50° C. Concentrated HCl (26 mL) and formaldehyde (3% aq, 36 mL) were added in portions during 30 min. The reaction mixture was stirred at 70° C. for 4.5 h. After cooling to rt, ammonia (aq, sat, 35 mL) was added to pH˜8. The precipitate was collected and washed with water, dried under vacuum, and the residue purified by chromatography, furnishing 8.33 g (53%) of intermediate I.

### Dimethyl 3,3′-methylenebis(6-acetamidobenzoate) II

Acetyl chloride (5.2 mL, 72.8 mmol), I (7.54 g, 24 mmol), triethylamine (10.08 mL, 72.8 mmol) was mixed in dioxane (160 mL) at 0° C. and stirred at rt for 22 h. The mixture was concentrated to a small volume and poured into water (200 mL). The precipitate was collected and washed with water. Drying gave intermediate II. Yield: 9.09 g (95%).

### Dimethyl 3,3′-carbonylbis(6-acetamidobenzoate) III

KMnO₄ (12 g, 76 mmol) was added in portions to a stirred mixture of compound II (9.0 g, 22.5 mmol), MgSO₄ (15% aq, 20 mL) and acetone (500 mL). After 8 d at rt, the mixture was filtered through celite and washed with CH₂Cl₂. The filtrate was washed with water, brine and MgSO₄ (aq, sat) and concentrated to afford 6.3 g (68%) of intermediate III.

### Dimethyl 3,3′-carbonylbis(6-aminobenzoate) IV

Compound III (6.0 g, 14.5 mmol) was dissolved in MeOH (600 mL) and HCl (aq, 5M, 540 mL). The mixture was stirred at rx for 1.5 h and concentrated to a smaller volume. NaHCO₃ was added to pH˜7-8 and the mixture extracted with EtOAc. The combined extracts where washed with brine and dried (MgSO₄) to afford product IV in 3.85 g (81%) yield.

### Procedure A for Aroylation of IV Producing dimethyl 3,3′-carbonylbis(6-aroylaminobenzoate) V

A mixture of compound IV (164 mg, 0.5 mmol), aroyl chloride (1.5 mmol) and toluene (10 mL) was heated at rx under inert atm for 4 h. The mixture was cooled and diluted with EtOAc. Extractive workup (NaHCO₃ (aq, sat) and brine) followed by drying (Na₂SO₄) and recrystallisation gave the esters V in yields given in table 1.

### Procedure B for Arylation of IV Producing dimethyl 3,3′-carbonylbis(6-arylaminobenzoate) V

Compound IV (210 mg, 0.64 mmol), aryl bromide (1.92 mmol), palladium acetate (5.8 mg, 0.0128 mmol), DPE-phos (20.6 mg, 0.0192 mmol), and cesium carbonate (0.875 g, 2.69 mmol) were mixed in dioxane (10 mL). The reaction vessel was sealed with a septa and the mixture stirred at rt for 5 min and at 95° C. for 22 h with extra addition of DPE-phos (20.6 mg) and palladium acetate (5.8 mg) after 16 h. After cooling to rt, the mixture was concentrated and water (60 mL) was added. Acidification with HCl (1M, aq) to pH˜2-3 and extractive workup using EtOAc furnished, after drying (Na₂SO₄), concentration and purification by chromatography the esters V.

### Procedure C for arylation of IV producing dimethyl 3,3′-carbonylbis(6-arylamino-benzoate) V

Compound IV (204 mg, 0.62 mmol), arylboronic acid (1.86 mmol), copper acetate (338 mg, 1.86 mmol), pyridine (147 mg, 1.86 mmol), triethylamine (0.188 g, 1.86 mmol), and molecular sieves (4 Å) were mixed in CH₂Cl₂ (10 mL) under dry conditions. The mixture was stirred at rt for 50 h with two extra additions (0.93 mmol) of all reagents (except IV) after 21 and 45 h respectively. After cooling to rt, the mixture was filtered through celite and washed with CH₂Cl₂. The solution was washed with NH₃ (aq), water, brine and dried (Na₂SO₄). After concentration and chromatography the esters V was obtained.

### Method D for the Preparation of Example 4 and 5 (Table 1)

The intermediate 5,5′-methylenebis(2-aminobenzoic acid) (A) is commercially available (e.g. Maybridge), but was prepared as described in the literature (Bioorg. Med. Chem. 2006, 14, 2209).

Intermediate A (250 mg, 0.873 mmol) was added in portions to a solution of sodium carbonate (466 mg, 2.18 mmol, in 5 mL of water) at 50° C. Arylsulfonyl chloride (2.18 mmol) was added in portions and the mixture was stirred at 70° C. for 30 min and then at 85° C. for additional 30 min. After cooling to rt the mixture was acidified with dilute HCl (aq). The precipitate was collected and washed with dilute HCl (aq) and then water to give the 5,5′-methylenebis(2-(arylsulfonamido)benzoic acid (intermediate B) compound as a solid.

### Preparation of 5,5′-carbonylbis(2-(arylsulfonamido)benzoic Acid) VI

To a solution of B (0.304 mmol) in acetone (10 mL) was added 15% (aq) solution of MgSO₄ (0.334 mmol, 0.270 mL). KMnO₄ (164 mg, 1.04 mmol) was added in portions and the resulting mixture stirred at rt for two days. The mixture was filtered and concentrated and the dark brown residue treated with NaOH (0.2M, aq). The brown solids were filtered off and the filtrate acidified with HCl (2M, aq). The precipitate was collected, washed with water and recrystallised from THF/n-hexane to afford the title compound VI.

### Method E for One-Pot di-sulfonylation of IV

Compound IV (0.46 g, 1.4 mmol) and DMAP (34 mg, 0.28 mmol) was dissolved in pyridine (28 mL) and cooled to 0° C. 4-Butoxybenzenesulfonyl chloride (1.045 g, 4.2 mmol) was added and the mixture stirred at rt for 38 h with addition of another portion of 4-butoxybenzenesulfonyl chloride (0.35 g, 1.4 mmol) after 14 h. The mixture was concentrated. Extractive workup (HCl (1M, aq), EtOAc) followed by drying (Na₂SO₄) concentration and purification by chromatography afforded pure mono- (220 mg) and disulfonylated product (213 mg, 19%). The disulfonylated product (0.16 g, 0.205 mmol) was hydrolysed according to the general procedure H affording the pure di-acid (109 mg, 73%).

### Procedure F for di-carbamoylation of IV Producing Inhibitor VI

Compound IV (1.0 g, 3.05 mmol) was dissolved in an aqueous solution of NaOH (1.22 g, 30.5 mmol) and 140 mL EtOH was added. The mixture was stirred at rx for 1.5 h then cooled and acidified with HCl (aq). The precipitate was collected, washed with water, dried and recrystallised from EtOH/water to give the free acid (0.4 g, 44%). The free acid (0.15 g, 0.5 mmol) was mixed with 4-trifluoromethyl-phenylisocyanate (206 mg, 1.1 mmol) in DMF (2 mL) under argon and stirred over night. A second portion of isocyanate was added (60 mg) and the mixture was stirred over nigh. Water (3 mL) was added and the precipitate collected. Recrystallisation afforded the pure compound VI (29 mg, 8.6%).

### Procedure G for Sequential di-aroylation of IV Producing, After Hydrolysis, Inhibitors IX as Depicted in Table 2

Compound IV (296 mg, 0.902 mmol), aroyl chloride (0.902 mmol) and triethyl amine (91.2 mg, 0.902) were dissolved in dioxane (30 mL) and heated at 55° C. for 100 min under inert atm. After cooling and concentration, dilution with EtOAc gave a precipitate that was collected and purified by chromatography furnished mono-aroylated compound VII. Compound VII (0.190 mmol) and aroyl chloride (0.209 mmol) were dissolved in toluene (30 mL) and heated at rx for 20 h under inert atm. Cooling of the reaction mixture and dilution with MeOH delivered VIII as a precipitate which was collected and hydrolysed (e.g. see general procedure H) which delivered di-aroylated inhibitors IX.

### General Procedure H for Hydrolysis of V and VIII Producing Inhibitors Depicted in Table 1 and 3

Compound V (0.15 mmol) and NaOH (60 mg, 1.5 mmol) were dissolved in water (2 mL) and EtOH (10 mL) and heated at 60° C. for 0.5 h. After cooling to 0° C. and addition of HCl (1M, aq) to obtain pH˜2, the precipitate was collected and recrystallised, delivering the inhibitors as free acids.

### General Procedure I for Aroylation of methyl 2-amino-5-(4-(4-butoxyphenyl-sulfonamido)-3-(methoxycarbonyl)benzoyl)benzoate and Subsequent Hydrolysis

Methyl 2-amino-5-(4-(4-butoxyphenylsulfonamido)-3-(methoxycarbonyl)benzoyl)benzoate (0.11 g, 0.198 mmol), prepared by procedure G, was mixed with aroyl chloride (0.218 mmol), dissolved in toluene and stirred at rt for 20 h. After concentration, MeOH was added and the precipitate was collected and purified by chromatography. The hydrolysis was performed according to the general procedure H affording the pure di-acid in yields depicted in table 3.

TABLE 1
Symmetric Compounds of Examples 1-7 using Procedure A-F

| | | | | Yield (%) | |
|---|---|---|---|---|---|
| | | | | | Acid |
| No | Chemical name | Method | Substrate | Ester V | VI |
| 1 | 2-(3,4-difluorophenylamino)-5-((4- | B | 4-bromo-1,2- | 22 | 57 |
| | (3,4-difluorophenylamino)-3- | | difluorobenzene | | |
| | carboxyphenylcarbonyl))benzoic | | | | |
| | acid | | | | |
| 2 | 2-(4-isopropoxyphenylamino)-5- | C | 4-isopropoxyphenyl- | 24 | 38 |
| | (4-(4-isopropoxyphenylamino)-3- | | boronic acid | | |
| | carboxyphenylcarbonyl)benzoic | | | | |
| | acid | | | | |
| 3 | 2-(4-butylbenzamido)-5-(4-(4- | A | 4-butylbenzoyl chloride | 75 | 63 |
| | butylbenzamido)-3-carboxy- | | | | |
| | phenylcarbonyl)benzoic acid | | | | |
| 4 | 2-((4-nitrophenyl)sulfonylamino)- | D | 4-nitrobenzene-1- | — | 12 |
| | 5-(3-carboxy-4-(((4-nitrophenyl)- | | sulfonyl chloride | | |
| | sulfonyl)amino)benzoyl)benzoic | | | | |
| | acid | | | | |
| 5 | 2-((3,4-dichlorophenyl)sulfonyl- | D | 3,4-dichlorobenzene-1- | — | 13 |
| | amino)-5-(3-carboxy-4-(((3,4- | | sulfonyl chloride | | |
| | dichlorophenyl)sulfonyl)amino)- | | | | |
| | benzoyl)benzoic acid | | | | |
| 6 | 2-[3-(4-trifluoromethylphenyl)- | F | 1-isocyanato-4-(tri- | — | 9 |
| | ureido]-5-{3-carboxy-4-[3-(4- | | fluoromethyl)benzene | | |
| | trifluoromethylphenyl)ureido]- | | | | |
| | benzoyl}benzoic acid | | | | |
| 7 | 2-(4-n-Butoxybenzene- | E | 4-butoxybenzene-1- | 19 | 73 |
| | sulfonylamino-5-[3-carboxy-4-(4- | | sulfonyl chloride | | |
| | n-butoxybenzenesulfonylamino)- | | | | |
| | benzoyl]benzoic acid | | | | |

TABLE 2
Final compounds (Examples 8 to 11) prepared via two-step aroylation
according to the general method G

| | | First | Second | Yield (%) | |
|---|---|---|---|---|---|
| No | Chemical name | substrate | substrate | VIII | IX |
| 8 | 2-(2-fluoro-4-(trifluoromethyl)benzamido)- | 4-(trifluoro- | 2-fluoro-4- | 97 | 41 |
| | 5-(4-(4-(tri- | methyl)benzoyl | (trifluoro- | | |
| | fluoromethyl)benzamido)-3- | chloride | methyl)benzoyl | | |
| | carboxyphenylcarbonyl)- | | chloride | | |
| | benzoic acid | | | | |
| 9 | 2-(4-butylbenzamido)-5-(4-(4- | 4-(trifluoro- | 4-butyl- | 76 | 52 |
| | (trifluoromethyl)benzamido)-3- | methyl)benzoyl | benzoyl | | |
| | carboxyphenylcarbonyl)- | chloride | chloride | | |
| | benzoic acid | | | | |
| 10 | 2-(2,4-dichlorobenzamido)-5- | 2,4-dichloro- | 2,4-dichloro- | Impure | 85 |
| | (4-(2,4-dichlorobenzamido)-3- | benzoyl | benzoyl | VII | |
| | carboxyphenylcarbonyl)- | chloride | chloride | was | |
| | benzoic acid | | | used | |
| 11 | 2-(4-(trifluoromethoxy)benzamido)- | 2,4-dichloro- | 4-(trifluoro- | 48 | 31 |
| | 5-(4-(2,4-dichloro- | benzoyl | methoxy)- | | |
| | benzamido)-3-carboxyphenyl- | chloride | benzoyl | | |
| | carbonyl)benzoic acid | | chloride | | |

TABLE 3
Inhibitors (Examples 12 and 13) prepared according to general method I

| | | | Yield (%) | |
|---|---|---|---|---|
| | | | es- | |
| No | Chemical name | Substrate | ter | acid |
| 12 | 5-[(3-Carboxy-4-(4-n-butoxy- | 2,3-dichlorobenzoyl | 69 | 46 |
| | benzenesulfonylamino)benzoyl)]-2- | chloride | | |
| | (2,3-dichlorobenzoylamino)benzoic | | | |
| | acid | | | |
| 13 | 5-[(3-Carboxy-4-(4-n-butoxy- | 4- | 73 | 59 |
| | benzenesulfonylamino)benzoyl)]-2- | isopropoxybenzoyl | | |
| | (4-isopropoxybenzoylamino)- | chloride | | |
| | benzoic acid | | | |

TABLE 4
Spectroscopic data of the compounds of Examples 1-13

| No | ¹H NMR (DMSO-d₆, 400 or 200 MHz), δ: |
|---|---|
| 1 | 10.0 (2H, br s) 8.31 (2H, d, J = 2.0 Hz) 7.81-7.76 (2H, m) 7.56-7.40 (4H, m) |
| | 7.22-7.16 (4H, m) |
| 2 | 11.4-11.0 (2H, br s,) 8.39 (2H, s) 7.63-7.60 (2H, m) 7.16-7.14 (4H, m) |
| | 6.98-6.92 (6H, m) 4.56 (2H, septet, J = 6.0 Hz) 1.26 (12H, d, J = 6.0 Hz) |
| 3 | 12.45 (2H, s) 8.90 (2H, d, J = 8.8 Hz) 8.45 (2H, d, J = 2.0 Hz) 8.08 (2H, dd, |
| | J = 8.8 and 2.0 Hz) 7.89-7.84 (4H, m) 7.48-7.38 (4H, m) 2.98 (4H, t, J = 7.7 Hz) |
| | 1.69-1.50 (4H, m) 1.43-1.23 (4H, m) 0.91 (6H, t, J = 7.3 Hz) |
| 4 | 12.23 (2H, br s), 8.43-8.35 (4H, m), 8.25-8.15 (6H, m), 7.89-7.81 (2H, m), |
| | 7.58 (2H, d, J = 8.7 Hz) |
| 5 | 11.85 (2H, br s), 8.26-8.18 (4H, m), 7.93-7.83 (6H, m), 7.57 (2H, d, J = 8.7 Hz) |
| 6 | 11.0-10.8 (2H, br s) 10.46 (2H, s) 8.60 (1H, d, J = 9.3 Hz) 8.38 (1H, d, J = 2.4 Hz) |
| | 7.98 (1H, dd, J = 9.3 2.4 Hz) 7.83-7.68 (8H, m) |
| 7 | 11.6-11.4 (2H, br s) 8.20 (2H, d, J = 2.1 Hz) 7.88-7.81 (6H, m) 7.59 (2H, d, |
| | J = 8.8 Hz) 7.10-7.04 (4H, m) 4.00 (4H, t, J = 6.4 Hz) 1.69-1.61 (4H, m) |
| | 1.43-1.32 (4H, m) 0.88 (6H, t, J = 7.3 Hz) |
| 8 | 12.61 (1H, s) 12.33 (1H, d, J = 4.9 Hz) 8.86-8.81 (2H, m) 8.43-8.40 (2H, m) |
| | 8.17-7.93 (8H, m) 7.81-7.77 (1H, m) |
| 9 | 12.54 (1H, s) 12.42 (1H, s) 8.91-8.81 (2H, m) 8.43-8.42 (2H, m) |
| | 8.18-7.86 (8H, m) 7.43-7.39 (2H, m) 2.66 (2H, t, J = 7.5 Hz) 1.65-1.51 (2H, m) |
| | 1.36-1.51 (2H, m) 0.89 (3H, t, J = 7.3 Hz) |
| 10 | 11.94 (2H, s) 8.75 (2H, d, J = 8.8 Hz) 8.39 (2H, d, J = 2.4 Hz) 8.07 (2H, dd, |
| | J = 8.8, 2.0 Hz) 7.84-7.78 (4H, m) 7.63 (2H, dd, J = 8.3, 2.0 Hz) |
| 11 | 12.52 (1H, s) 11.97 (1H, s) 8.84 (1H, d, J = 8.8 Hz) 8.74 (1H, d, J = 8.3 Hz) |
| | 8.43-8.38 (2H, m) 8.12-8.04 (4H, m) 7.84-7.60 (5H, m) |
| 12 | 11.83 (1H, s) 11.6-11.5 (1H, br s) 8.66 (1H, d, J = 8.8 Hz) 8.31 (1H, d, J = 2.0 Hz) |
| | 8.26 (1H, d, J = 2.0 Hz) 8.01-7.91 (2H, m) 7.87-7.81 (3H, m) 7.71 (1H, dd, |
| | J = 7.8, 1.5 Hz) 7.59 (1H, d, J = 8.8 Hz) 7.57-7.49 (1H, m) 7.10-7.05 (2H, m) |
| | 4.00 (2H, t, J = 6.3 Hz) 1.69-1.58 (2H, m) 1.43-1.32 (2H, m) 0.87 (3H, t, J = 7.3 Hz) |
| 13 | 12.34 (1H, s) 11.7-11.6 (1H, br s) 8.84 (1H, d, J = 8.8 Hz) 8.36 (1H, d, J = 2.0 Hz) |
| | 8.26 (1H, d, J = 2.0 Hz) 7.99-7.83 (6H, m) 7.63 (1H, d J = 8.9 Hz) |
| | 7.12-7.06 (4H, m) 4.75 (1H, septet, J = 6.1 Hz) 4.01 (2H, t, J = 6.4 Hz) 1.70-1.59 (2H, m) |
| | 1.43-1.32 (2H, m) 1.29 (6H, d, J = 6.1 Hz) 0.88 (3H, t, J = 7.3 Hz) |

### Examples 14 to 19

### Preparation of Starting Materials and Active Inhibitors

### Methyl 2-hydroxy-5-(4-nitrobenzoyl)benzoate X

AlCl₃ (9.06 g, 68 mmol) was stirred in nitrobenzene (34 mL) at 0° C. under dry and inert conditions. Methyl 2-hydroxybenzoate (5.17 g, 34 mmol) was added to the mixture. 4-Nitrobenzoyl chloride (6.43 g, 34.66 mmol) was added in portions while maintaining the temperature at 0° C. The reaction mixture was heated at 100° C. for 1.5 h. After cooling and acidification using HCl (2M, aq), extractive workup (EtOAc, brine), drying of the combined extracts (Na₂SO₄) afforded the crude product after concentration. Recrystallisation of the crude in EtOAc afforded X (3.691 g, 36%).

### Methyl 5-(4-nitrobenzoyl)-2-(trifluoromethylsulfonyloxy)benzoate XI

X (3.31 g, 11 mmol) was dissolved in CH₂Cl₂ (120 mL) and pyridine (1.91 mL) at 0° C. Triflic anhydride (3.72 g, 13.2 mmol) was added in portions at 0° C. during 20 min. The reaction was allowed to slowly reach rt. The reaction mixture was diluted with EtOAc (360 mL) and 200 mL HCl (0.1M, aq) was added. Extractive workup (EtOAc, NaHCO₃ (aq, sat), brine) with subsequent drying of the extracts (Na₂SO₄) and concentration in vacuo afforded the pure product XI after chromatography (3.9 g, 82%).

### Methyl 2-(3,4-difluorophenylamino)-5-(4-nitrobenzoyl)benzoate XII

XI (0.660 g, 1.523 mmol), 3,4-difluoroaniline (0.236 g, 1.83 mmol), Cs₂CO₃ (0.695 g, 2.132 mmol), Pd(OAc)₂ (17.06 mg, 0.076 mmol), and rac-BINAP (71 mg, 0.114 mmol) were dissolved in toluene and heated at 100° C. under stirring and inert atmosphere for 20 h. After cooling of the reaction mixture, filtration through celite, washing of the precipitate with EtOAc, the crude was isolated after concentration of the filtrate. Recrystallisation (CH₂Cl₂) afforded XII, (399 mg, 63%).

### Methyl 5-(4-aminobenzoyl)-2-(3,4-difluorophenylamino)benzoate XIII

XII (0.288 g, 0.7 mmol), iron (0.585 g, 10.5 mmol) and ammonium chloride (15 mL, aq, sat) were dissolved in dioxane (20 mL) and isopropyl alcohol (30 mL). The mixture was heated at rx for 2.5 h. After cooling the mixture was filtered through Celite® and washed with EtOAc. Extractive workup of the filtrate (EtOAc, water, brine), drying of the combined extracts (Na₂SO₄) and concentration afforded after chromatography XIII (144 mg, 53%).

### Methyl 2-((3,4-difluorophenyl)(methyl)amino)-5-(4-nitrobenzoyl)benzoate XIV

XI (1.04 g, 2.4 mmol), 3,4-difluoro-N-methylaniline (0.412 g, 2.88 mmol), cesium carbonate (1.1 g, 3.36 mmol), palladium acetate (27 mg, 0.12 mmol) and rac-BINAP (0.112 g, 0.18 mmol) were dissolved in toluene, stirred and heated under inert atmosphere at 100° C. during 22 h. Cooling of the reaction mixture, filtration and washing (EtOAc) through Celite® furnished after concentration of the filtrate the crude which after chromatography delivered the pure compound XIV (910 mg, 88%).

### Methyl 5-(4-aminobenzoyl)-2-((3,4-difluorophenyl)(methyl)amino)benzoate XV

XIV (0.90 g, 2.11 mmol), iron (1.77 g, 31.66 mmol) and ammonium chloride (80 mL, aq, sat) were dissolved in isopropyl alcohol (100 mL). The mixture was heated at rx for 1.5 h. After cooling the mixture was filtered through Celite® and washed with EtOAc. Concentration of the filtrate and extractive workup (EtOAc, water, brine), drying of the combined extracts (Na₂SO₄) and concentration afforded after chromatography XV (803 mg, 95%).

### Method J for Aryl Sulfonylation of XIII

Aryl sulfonyl chloride (0.221 mmol) and XIII (77 mg, 0.201 mmol) were dissolved in pyridine (8 mL) at 0° C. and the mixture was stirred at rt for 7 h. Extractive workup (EtOAc, water, HCl (0.5M, aq), brine), drying of the combined extracts (Na₂SO₄) and concentration afforded after chromatography methyl 5-(4-(4-arylsulfonamido)benzoyl)-2-((3,4-difluorophenyl)amino)benzoate. Hydrolysis according to general method H (see above) furnished the free acid (see table 5).

### Method K for Aroylation of XIII

XIII (71 mg, 0.185 mmol) and aroyl chloride (0.204 mmol) were dissolved in toluene under an inert atmosphere and heated at rx for 4 h. The reaction was quenched by the addition of methanol (5 mL) and stirred for 10 min. Concentration and chromatography of the residue afforded methyl 2-(3,4-difluorophenylamino)-5-(4-(arylamido)benzoyl)benzoate. Hydrolysis according to general method H furnished the free acid (see table 5).

### Method L for Arylation of XIII

XIII (0.144 g, 0.376 mmol), aryl bromide (0.452 mmol), cesium carbonate (172 mg, 0.527 mmol), palladium acetate (4.2 mg, 0.018 mmol) and rac-BINAP (17.2 mg, 0.0277 mmol) were dissolved in toluene (2.8 mL), stirred and heated under inert atmosphere at 100° C. during 20 h. Cooling of the reaction mixture, filtration and washing (EtOAc) through Celite® furnished after concentration of the filtrate the crude which after chromatography delivered the pure compound methyl 5-(4-(arylamino)benzoyl)-2-(3,4-difluorophenylamino)benzoate. Hydrolysis according to general method H furnished the free acid (see table 5).

### Method M for Aryl Sulfonylation of XV

Aryl sulfonyl chloride (0.333 mmol) and XV (120 mg, 0.303 mmol) were dissolved in pyridine (8 mL) at 0° C. and the mixture was stirred at rt for 6 h. Concentration of the reaction mixture and subsequent extractive workup (EtOAc, water, HCl (0.5M, eq), brine), drying of the combined extracts (Na₂SO₄) and concentration afforded after chromatography methyl 5-(4-(4-arylsulfonamido)benzoyl)-2-((3,4-difluorophenyl)(methyl)amino)benzoate. Hydrolysis according to general method H furnished the free acid (see table 5).

### Method N for Aroylation of XV

XV (120 mg, 0.303 mmol) and aroyl chloride (0.333 mmol) were dissolved in toluene (12 mL), put under inert atmosphere and heated at rx for 0.5 h. The reaction was quenched by addition of methanol (5 mL) and stirring for 10 min. Concentration and chromatography of the residue afforded methyl 5-(4-(arylamido)benzoyl)-2-(3,4-difluorophenyl)(methyl)amino)benzoate. Hydrolysis according to general method H furnished the free acid (see table 5).

### Method O for Arylation of XV

XV (0.180 g, 0.454 mmol), aryl bromide (0.545 mmol), cesium carbonate (207 mg, 0.636 mmol), palladium acetate (5.1 mg, 0.0225 mmol) and rac-BINAP (21 mg, 0.0377 mmol) were dissolved in toluene (3.4 mL), stirred and heated under inert atmosphere at 100° C. during 16 h. Cooling of the reaction mixture, filtration and washing (EtOAc) through Celite® furnished after concentration of the filtrate the crude mixture, which after chromatography delivered pure methyl 5-(4-(arylamino)benzoyl)-2-(3,4-difluorophenyl)(methyl)amino)benzoate. Hydrolysis according to general method H furnished the free acid.

TABLE 5
Examples 14 to 19

| | | Starting | | | |
|---|---|---|---|---|---|
| | | material/ | | Yield % | |
| No | Chemical name | method | Substrate | Ester | Acid |
| 14 | 5-(4-(4-butoxyphenylsulfonamido)benzoyl)- | XIII/J | 4-butoxybenzene- | 92 | 91 |
| | 2-(3,4- | | 1-sulfonyl | | |
| | difluorophenylamino)benzoic | | chloride | | |
| | acid | | | | |
| 15 | 2-(3,4- | XIII/K | 4-isopropoxy- | 96 | 65 |
| | difluorophenylamino)-5-(4- | | benzoyl chloride | | |
| | (4-isopropoxybenzamido)benzoyl)benzoic | | | | |
| | acid | | | | |
| 16 | 5-(4-(4-chlorophenylamino)- | XIII/L | 1-bromo-4- | 34 | 76 |
| | benzoyl)-2-(3,4-difluoro- | | chlorobenzene | | |
| | phenylamino)benzoic acid | | | | |
| 17 | 5-(4-(4-butoxyphenylsulfonamido)benzoyl)- | XV/M | 4-butoxybenzene- | 75 | 94 |
| | 2-((3,4-di- | | 1-sulfonyl | | |
| | fluorophenyl)(methyl)amino)benzoic | | chloride | | |
| | acid | | | | |
| 18 | 5-(4-(5-chloro-2-hydroxy- | XV/N | 4-chloro-2- | 90 | 48 |
| | benzamido)benzoyl)-2- | | (chloro- | | |
| | ((3,4- | | carbonyl)phenyl | | |
| | difluorophenyl)(methyl)- | | acetate | | |
| | amino)benzoic acid | | | | |
| 19 | 5-(4-(4-chlorophenylamino)- | XV/O | 1-bromo-4- | 51 | 48 |
| | benzoyl)-2-((3,4-difluoro- | | chlorobenzene | | |
| | phenyl)(methyl)amino)- | | | | |
| | benzoic acid | | | | |

TABLE 6
Spectroscopic Data of the Compounds of Examples 14 to 19

| Example | |
|---|---|
| No | ¹H NMR (DMSO-d₆, 200 MHz), δ: |
| 14 | 13.7-13.2 (1H, br s) 10.71 (1H, s) 10.05 (1H, s) 8.25 (1H, d, J = 2.0 Hz) |
| | 7.79-7.69 (3H, m) 7.63-7.54 (2H, m) 7.54-7.41 (2H, m) 7.27-7.00 (6H, |
| | m) 3.99 (2H, t, J = 6.3 Hz) 1.71-1.57 (2H, m) 1.45-1.30 (2H, m) |
| | 0.88 (3H, t, J = 7.3 Hz) |
| 15 | 13.8-13.1 (1H, br s) 10.38 (1H, s) 10.1-10.0 (1H, br s) 8.34 (1H, d, |
| | J = 2.4 Hz) 8.00-7.90 (4H, m) 7.82 (1H, dd, J = 8.8 and 2.0 Hz) |
| | 7.75-7.67 (2H, m) 7.56-7.39 (2H, m) 7.24-7.15 (2H, m) 7.08-6.99 (2H, m) |
| | 4.74 (1H, septet, J = 5.9 Hz) 1.28 (6H, d, J = 5.9 Hz) |
| 16 | 13.8-13.0 (1H, br s) 10.2-9.9 (1H, br s) 8.92 (1H, s) 8.30 (1H, d, J = 2.0 Hz) |
| | 7.77 (1H, dd, J = 8.8 and 2.0 Hz) 7.69-7.59 (2H, m) 7.57-7.41 (2H, |
| | m) 7.40-7.29 (2H, m) 7.26-7.14 (4H, m) 7.14-7.04 (2H, m) |
| 17 | 13.1-12.7 (1H, br s) 10.9-10.6 (1H, br s) 7.94 (1H, d, J = 2.0 Hz) |
| | 7.83 (1H, dd, J = 8.3 and 2.0) 7.79-7.71 (2H, m) 7.71-7.61 (2H, m) 7.42 (1H, |
| | d, J = 8.3 Hz) 7.29-7.13 (3H, m) 7.12-7.01 (2H, m) 6.86-6.69 (1H, m) |
| | 6.51-6.39 (1H, m) 3.99 (2H, t, J = 6.3 Hz) 3.25 (3H, s) 1.73-1.55 (2H, m) |
| | 1.47-1.27 (2H, m) 0.88 (3H, t, J = 7.3 Hz) |
| 18 | 13.3-12.3 (1H, br s) 12.2-11.1 (1H, br s) 10.69 (1H, s) 8.03 (1H, d, |
| | J = 2.0 Hz) 7.97-7.92 (1H, m) 7.92-7.86 (3H, m) 7.85-7.76 (2H, m) |
| | 7.46 (2H, dd, J = 8.8 and 2.4 Hz) 7.29-7.12 (1H, m) 7.02 (1H, d, J = 8.8 Hz) |
| | 6.88-6.73 (1H, m) 6.53-6.42 (1H, m) 3.28 (3H, s) |
| 19 | 13.0-12.9 (1H, br s) 9.00 (1H, s) 7.98 (1H, d, J = 2.0 Hz) 7.88 (1H, dd, |
| | J = 8.3 and 2.0 Hz) 7.53-7.65 (2H, m) 7.45 (1H, d, J = 8.3 Hz) |
| | 7.40-7.30 (2H, m) 7.25-7.07 (5H, m) 6.83-6.66 (1H, m) 6.48-6.36 (1H, m) |
| | 3.26 (3H, s) |

### Preparation of methyl 5-(4-((4-chlorophenyl)(methyl)amino)benzoyl)-2-fluorobenzoate XVI

Step 1: 2-Fluoro-5-iodobenzoic acid (25.15 g, 94.5 mmol), Me₂SO₄ (12.9 g, 102 mmol) and K₂CO₃ (14.36 g, 104 mmol) were dissolved in DMF (25 mL) and stirred at 150° C. for 2 h. The reaction mixture was cooled to rt, diluted with water and extracted (EtOAc). The combined extracts were washed with water, dried (Na₂SO₄) and concentrated. The crude was purified by chromatography to furnish methyl 2-fluoro-5-iodobenzoate (9.11 g, 34%).

Step 2: Methyl 2-fluoro-5-iodobenzoate (5.231 g, 18.68 mmol) was dissolved in dry THF and cooled to −30° C. i-PrMgCl (sol. in THF, ˜0.8M, 33.53 mL) was added dropwise while maintaining the temperature. The reaction mixture was stirred for 1 h and then added to a cooled (−60° C.) THF solution of 4-bromobenzoyl chloride. The resulting solution was stirred at −40° C. for 4 h. Extractive workup (EtOAc, water, brine, K₂CO₃ (aq, sat)) with drying (Na₂SO₄) of the combined extracts, gave after concentration the crude which was recrystallized in an appropriate solvent to furnish methyl 5-(4-bromobenzoyl)-2-fluorobenzoate (3.32 g, 53%).

Step 3. Methyl 5-(4-bromobenzoyl)-2-fluorobenzoate was reacted with 4-chloro-N-methylaniline according to method L to furnish methyl 5-(4-((4-chlorophenyl)(methyl)amino)benzoyl)-2-fluorobenzoate XVI (2.216 g, 57%).

### General Method for Etherification of XVI

XVI (0.2 g, 0.5 mmol, 1 equiv), aryl alcohol (1 equiv), potassium fluoride on aluminium oxide (2 equiv), and 18-crown-6-ether (1 equiv) was dissolved in CH₃CN (3 mL) and heated at rx for 20 h. Extractive workup (EtOAc, water, brine, HCl (aq, 0.1M)) with drying (Na₂SO₄) and concentration of the combined extracts gave the crude which was purified by chromatography to furnish methyl 2-(aryloxy)-5-(4-((4-chlorophenyl)(methyl)amino)benzoyl)benzoate. Subsequent hydrolysis according to procedure H furnished the inhibitors depicted in table 8.

### Preparation of methyl 5-(4-aminobenzoyl)-2-fluorobenzoate

Step 1: Methyl 2-fluoro-5-iodobenzoate (4.3 g, 15.4 mmol) was dissolved in dry THF and cooled to −30° C. i-PrMgCl (sol. in THF, 1M, 21.5 mL) was added dropwise while maintaining the temperature and stirring for 1 h. The mixture was cooled to −78° C. and then added to a solution of 4-nitrobenzoyl chloride (5.72 g, 31 mmol) in THF (20 mL) at −78° C. The resulting mixture was allowed to reach rt before NaHCO₃ (aq, sat) was added. After 10 min stirring, extractive workup (EtOAc, NaHCO₃ (aq, sat)), drying (Na₂SO₄) of the combined extracts, and concentration furnished methyl 2-fluoro-5-(4-nitrobenzoyl)benzoate (2.10 g).

Step 2: Methyl 2-fluoro-5-(4-nitrobenzoyl)benzoate (2.05 g, 6.76 mmol), iron (1.89 g, 33.8 mmol) and iron trichloride hexahydrate (9.12 g, 33.8 mmol) were dissolved in ethanol/water (80/20 v/v) and heated at rx for 3 h. The resulting mixture was filtered through Celite, washed with EtOAc and concentrated. Extractive workup (EtOAc, water, brine) with drying (Na₂SO₄) and concentration of the combined extracts, afforded the crude product. Recrystallization in ethanol delivered the pure methyl 5-(4-aminobenzoyl)-2-fluorobenzoate (1.9 g).

### Preparation of methyl 2-amino-5-(4-(3-chlorobenzamido)benzoyl)benzoate XVII

Step 1: Methyl 5-(4-aminobenzoyl)-2-fluorobenzoate was aroylated with 3-chlorobenzoyl chloride, according to general method K, furnishing methyl 5-(4-(3-chlorobenzamido)benzoyl)-2-fluorobenzoate.

Step 2: Methyl 5-(4-(3-chlorobenzamido)benzoyl)-2-fluorobenzoate (1.49 g, 3.61 mmol) and NaN₃ (0.47 g, 7.24 mmol) were dissolved in DMSO (50 mL) and stirred at 70° C. for 48 h. The reaction mixture was poured into water and subsequent extractive workup (EtOAc, brine) with drying (Na₂SO₄) and concentration of the combined extracts, afforded the crude product which was purified by chromatography to yield methyl 2-azido-5-(4-(3-chlorobenzamido)-benzoyl)benzoate (0.78 g).

Step 3: Methyl 2-azido-5-(4-(3-chlorobenzamido)benzoyl)benzoate (0.78 g, 1.79 mmol), Zn (0.176 g, 2.69 mmol) and iron trichloride hexahydrate (0.727 g, 2.69 mmol) were mixed in ethanol (50 mL) and heated at rt for 2 h. Cooling to rt, filtration through Celite, and washing with hot dioxane gave the crude product after concentration. Extractive workup (EtOAc, water, brine) with drying (Na₂SO₄) and concentration of the combined extracts, furnished a residue which was recrystallized in ethanol to afford the pure methyl 2-amino-5-(4-(3-chlorobenz-amido)benzoyl)benzoate XVII (0.508 g).

### Preparation of methyl 2-amino-5-(4-(4-chlorophenylsulfonamido)benzoyl)-benzoate XVIII

Step 1: Methyl 5-(4-aminobenzoyl)-2-fluorobenzoate was sulfonylated with 4-chlorobenzenesulfonyl chloride, according to general method J, furnishing methyl 5-(4-(4-chlorophenylsulfonamido)benzoyl)-2-fluorobenzoate.

Step 2: Methyl 5-(4-(4-chlorophenylsulfonamido)benzoyl)-2-fluorobenzoate was reacted according to step 2 and 3 in the preparation of XVII furnishing methyl 2-amino-5-(4-(4-chlorophenylsulfonamido)benzoyl)benzoate XVIII.

### Synthesis of methyl 5-(4-aminobenzoyl)-2-(aryloxy)benzoate XIX

Step 1: Methyl 5-(4-nitrobenzoyl)-2-(trifluoromethylsulfonyloxy)benzoate XI (0.1 g, 0.231 mmol, 1 equiv), aryl alcohol (1.2 equiv), K₃PO₄ (0.098 g, 0.462 mmol), Pd(OAc)₂ (1.04 mg, 0.0046 mmol) and biphenyl-2-yldi-tert-butylphosphine (2 mg, 0.0069 mmol) were dissolved in toluene (2 mL) and heated at 100° C. for 19 h under inert atmosphere. After cooling, and filtration through celite including washing with EtOAc, the resulting solution was concentrated. The residue was purified by chromatography to afford methyl 2-(aryloxy)-5-(4-nitrobenzoyl)benzoate.

Step 2: Methyl 2-(aryloxy)-5-(4-nitrobenzoyl)benzoate was reduced according to synthesis of XIII furnishing methyl 5-(4-aminobenzoyl)-2-(aryloxy)benzoate XIX.

### Synthesis of methyl 5-(4-bromobenzoyl)-2-(aryloxy)benzoate XX

Methyl 5-(4-bromobenzoyl)-2-fluorobenzoate (4.36 g, 12.93 mmol, 1 equiv), aryl alcohol (1 equiv), KF.Al₂O₃ (2 equiv) and 18-crown-6-ether (0.1 equiv) were dissolved in dry acetonitrile (30 mL) and heated at reflux temperature for 20 h. Extractive workup (EtOAc, HCl (aq, 1M), water, brine) afforded, after drying (Na₂SO₄) and concentration of the combined extracts, a residue which was purified by chromatography to deliver methyl 5-(4-bromobenzoyl)-2-(aryloxy)benzoate XX.

### General Method P for Carbamoylation

Aryl amine (0.444 mmol, 1 equiv) and aryl isocyanate (1.2 equiv) was dissolved in dioxane (10 mL) and stirred at rt for a few days until full conversion of starting material was achieved. The mixture was concentrated affording the crude which was purified by chromatography to furnish the inhibitors in table 7, after subsequent hydrolysis according to procedure H.

### Method Q for Amidation

Aryl bromide (0.22 mmol, 1 equiv), aryl amide (1.2 equiv), CuI (0.1 equiv), N1,N2-dimethylethane-1,2-diamine (0.2 equiv) and K₃PO₄ (2.2 equiv) were dissolved in dioxane (3 mL) under inert atmosphere and stirred at rx for 18 h. The reaction mixture was filtered through celite and the residue purified by chromatography. Subsequent hydrolysis according to procedure H afforded the depicted example in table 10.

### Method R for Etherification

Methyl 2-(aryloxy)-5-(4-iodobenzoyl)benzoate XXb (prepared as in the preparation of XX using 4-iodobenzoyl chloride in the description for preparation of XVI (step 2)) (0.2 g, 0.405 mmol, 1 equiv), aryl alcohol (1.5 equiv), CuI (0.05 equiv), N,N-dimethyl glycine.HCl (0.2 equiv), and Cs₂CO₃ (2 equiv) were dissolved under inert atmosphere in dioxane (2.5 mL) and stirred at 100° C. for 40 h. Cooling to rt, filtration through celite, washing (EtOAc) and concentration afforded the crude, which was purified by chromatography to furnish methyl 2-(aryloxy)-5-(4-aryloxybenzoyl)benzoate. Subsequent hydrolysis according to procedure H afforded the depicted example in table 10.

### Preparation of 5-{4-[(4-Chloro-phenyl)(methyl)amino]-benzoyl}-2-(arylamino)-benzoic Acid XXI

Methyl 5-(4-bromobenzoyl)-2-hydroxybenzoate was prepared according to the procedure for synthesis of X substituting 4-nitrobenzoyl chloride with 4-bromobenzoyl chloride.

Step 1: Methyl 5-(4-bromobenzoyl)-2-hydroxybenzoate (7.55 g, 22.5 mmol), Me₂SO₄ (3.13 g, 24.8 mmol), and K₂CO₃ (3.42 g, 24.8 mmol) were mixed in DMF (56 mL) under dry conditions and heated at 60° C. until complete conversion was obtained. The reaction mixture was cooled and concentrated. Extractive workup (EtOAc, NaHCO₃, (5%, aq), water, brine) with drying (Na₂SO₄) and concentration of the organic extracts furnished after recrystallisation (EtOH) pure methyl 5-(4-bromobenzoyl)-2-methoxybenzoate (7.11 g, 90%).

Step 2: Coupling of methyl 5-(4-bromobenzoyl)-2-methoxybenzoate with 4-chloro-N-methylaniline according to method L furnished methyl 5-(4-((4-chlorophenyl)(methyl)amino)benzoyl)-2-methoxybenzoate (71%).

Step 3: Methyl 5-(4-((4-chlorophenyl)(methyl)amino)-benzoyl)-2-methoxy-benzoate (1.54 g, 3.76 mmol) was dissolved in dichloromethane (88 mL), cooled to −20° C. and mixed with BBr₃ (3.77 g in 44 mL CH₂Cl₂). Stirring was maintained at −20° C. for 0.5 h. Dry MeOH (120 mL) was added and the mixture stirred for 0.5 h. Concentration and extractive workup (EtOAc, water, brine) with drying (Na₂SO₄) and concentration of the organic extracts furnished, after purification by chromatography, pure methyl 5-(4-((4-chlorophenyl)(methyl)amino)benzoyl)-2-hydroxybenzoate (0.919 g, 61%).

Step 4: Triflatation as in the preparation of XI furnished methyl 5-(4-((4-chlorophenyl)(methyl)amino)benzoyl)-2-(trifluoromethylsulfonyloxy)benzoate (82%)

Step 5: Methyl 5-(4-((4-chlorophenyl)(methyl)amino)benzoyl)-2-(trifluoromethyl-sulfonyloxy)benzoate was reacted with an aryl amine (see table 11) as in the preparation of XII and hydrolysed according to procedure H to furnish the inhibitors depicted in table 11.

### Synthesis of Inhibitors in Table 12

Step 1: 2-Fluoro-5-iodobenzonitrile (2.0 g, 7.3 mmol) was dissolved in THF (13 mL) and cooled to −35° C., then i-PrMgCl (sol. in THF, 1.5M, 7.3 mL) was slowly added while maintaining the temperature. The mixture was stirred at −25° C. for 1 h and then transferred to a cooled (−70° C.) THF (9 mL) solution of 4-bromobenzoyl chloride (3.20 g, 14.6 mmol). After 1 h stirring at −70° C. the mixture was allowed to slowly attain rt. Concentration and addition of water (100 mL) gave a slurry that was neutralised. Extractive workup (EtOAc, NaHCO₃ (aq, sat), brine), drying (Na₂SO₄) of the combined extracts and concentration furnished, after purification by chromatography, 5-(4-bromobenzoyl)-2-fluorobenzonitrile (1.53 g, 69%).

Step 2: 5-(4-bromobenzoyl)-2-fluorobenzonitrile (1.50 g, 4.93 mmol), and 4-chloro-N-methyl aniline (0.98 g, 6.9 mmol) was coupled according to method O and furnished 64% of 5-(4-((4-chlorophenyl)(methyl)amino)benzoyl)-2-fluorobenzonitrile.

Step 3: 5-(4-((4-Chlorophenyl)(methyl)amino)benzoyl)-2-fluorobenzonitrile was coupled with the depicted alcohol according to synthesis of XX (ex 10:1-3), and method K (ex 10:4).

Step 4: The product from step 3 (0.36 mmol, 1 equiv) was mixed with NaN₃ (3 equiv), triethyl ammonium chloride (3 equiv) and dissolved in toluene (4 mL). The mixture was heated in a sealed vial at 130° C. for 18 h. Cooling and extractive workup (EtOAc, NaOH (2M, aq), HCl (2M, aq)) with drying (Na₂SO₄) and concentration of the combined extracts, delivered the final products depicted in table 12.

### Synthesis of Inhibitors in Table 13

Step 1: Dimethyl 5,5′-methylenebis(2-aminobenzoate) (5.00 g, 15.9 mmol) was added to 15.4 mL HBr (48%, aq) under stirring. A solution of NaNO₂ (2.28 g in 5.6 mL water) was added dropwise at 0° C. The resulting solution was added dropwise to a warm (90° C.) solution of CuBr (4.30 g, 29.97 mmol) in HBr (48%, aq). The heating (90° C.) was maintained for 10 min before cooling and addition of diethyl ether. Extractive workup (diethyl ether), washing of the combined extracts (brine) and drying (Na₂SO₄), afforded after concentration and chromatography dimethyl 5,5′-methylenebis(2-bromobenzoate) (4.37 g, 60%).

Step 2: Dimethyl 5,5-methylenebis(2-bromobenzoate) was oxidized in a similar manner as in the synthesis of III furnishing dimethyl 5,5′-carbonylbis-(2-bromobenzoate) (38%).

Step 3: Dimethyl 5,5′-carbonylbis(2-bromobenzoate) were coupled using a similar protocol as in method L (11:1-2). Example 11: 3 was prepared using the method in the preparation of XIX (step 1) to couple dimethyl 5,5′-carbonylbis-(2-bromobenzoate) with the aryl alcohol.

Step 4: Examples 11: 1-3 were hydrolysed according to procedure H.

TABLE 7
Inhibitors prepared via procedure K, L and P employing starting material
XIII

| | | | | Yield (%) | |
|---|---|---|---|---|---|
| No | Chemical name | Method | Substrate | ester | acid |
| 5:1 | 5-{4-[3-(3-Chloro-phenyl)- | P | 1-Chloro-3- | 58 | 63 |
| | ureido]-benzoyl}-2-(3,4- | | isocyanatobenzene | | |
| | difluoro-phenylamino)-benzoic | | | | |
| | acid | | | | |
| 5:2 | 2-(3,4-Difluoro-phenylamino)- | P | 1-Ethoxy-4- | 66 | 71 |
| | 5-{4-[3-(4-ethoxy-phenyl)- | | isocyanatobenzene | | |
| | ureido]-benzoyl}-benzoic acid | | | | |
| 5:3 | 2-(3,4-Difluoro-phenylamino)- | L | 4-Bromo-1,2- | 45 | 65 |
| | 5-[4-(3,4-difluoro- | | difluorobenzene | | |
| | phenylamino)-benzoyl]- | | | | |
| | benzoic acid | | | | |
| 5:4 | 5-[4-(5-Chloro-2-hydroxy- | K | 4-Chloro-2- | Crude | 65 |
| | benzoylamino)-benzoyl]-2-(3,4- | | (chlorocarbonyl)- | mixture | |
| | difluoro-phenylamino)-benzoic | | phenyl acetate | | |
| | acid | | | | |
| 5:5 | 5-[4-(2-Chloro-benzoylamino)- | K | 2-Chlorobenzoyl | 85 | 96 |
| | benzoyl]-2-(3,4-difluoro- | | chloride | | |
| | phenylamino)-benzoic acid | | | | |
| 5:6 | 5-[4-(5-Chloro-pyridin-2- | L | 2-Bromo-5- | 56 | 82 |
| | ylamino)-benzoyl]-2-(3,4- | | chloropyridine | | |
| | difluoro-phenylamino)-benzoic | | | | |
| | acid | | | | |
| 5:7 | 5-[4-(6-Chloro-pyridin-3- | L | 5-Iodo-2- | Crude | 89 |
| | ylamino)-benzoyl]-2-(3,4- | | chloropyridine | mixture | |
| | difluoro-phenylamino)-benzoic | | | | |
| | acid | | | | |
| 5:8 | 5-[4-(5-Chloro-2-methoxy- | K | 5-Chloro-2- | 82 | 67 |
| | benzoylamino)-benzoyl]-2-(3,4- | | methoxybenzoyl | | |
| | difluoro-phenylamino)-benzoic | | chloride | | |
| | acid | | | | |
| 5:9 | 5-[4-(4-Chloro-2-methoxy- | K | 4-Chloro-2- | 94 | 85 |
| | benzoylamino)-benzoyl]-2-(3,4- | | methoxybenzoyl | | |
| | difluoro-phenylamino)-benzoic | | chloride | | |
| | acid | | | | |
| 5:10 | 5-[4-(2,5-Dichloro- | K | 2,5- | 80 | 82 |
| | benzoylamino)-benzoyl]-2-(3,4- | | Dichlorobenzoyl | | |
| | difluoro-phenylamino)-benzoic | | chloride | | |
| | acid | | | | |
| 5:11 | 5-[4-(3-Chloro-benzoylamino)- | K | 3-Chlorobenzoyl | 97 | 64 |
| | benzoyl]-2-(3,4-difluoro- | | chloride | | |
| | phenylamino)-benzoic acid | | | | |
| 5:12 | 5-{4-[(2,5-Dichloro-pyridine-3- | K | 3,6- | 68 | 42 |
| | carbonyl)-amino]-benzoyl}-2- | | Dichloropicolinoyl | | |
| | (3,4-difluoro-phenylamino)- | | chloride | | |
| | benzoic acid | | | | |

TABLE 8
Inhibitors prepared via General method for etherification of XVI and
subsequent hydrolysis according to method H

| | | | Yield (%) | |
|---|---|---|---|---|
| No | Chemical name | Substrate | ester | acid |
| 6:1 | 2-(4-Chloro-phenoxy)-5-{4-[(4- | 4-Chlorophenol | 63 | 59 |
| | chloro-phenyl)-methyl-amino]- | | | |
| | benzoyl}-benzoic acid | | | |
| 6:2 | 2-(3-Chloro-phenoxy)-5-{4-[(4- | 3-Chlorophenol | 87 | 49 |
| | chloro-phenyl)-methyl-amino]- | | | |
| | benzoyl}-benzoic acid | | | |
| 6:3 | 5-{4-[(4-Chloro-phenyl)-methyl- | 3,4-Difluoro-phenol | 85 | 45 |
| | amino]-benzoyl}-2-(3,4-difluoro- | | | |
| | phenoxy)-benzoic acid | | | |
| 6:4 | 2-(2-Chloro-phenoxy)-5-{4-[(4- | 2-Chloro-phenol | 66 | 91 |
| | chloro-phenyl)-methyl-amino]- | | | |
| | benzoyl}-benzoic acid | | | |
| 6:5 | 2-(4-Chloro-2-methoxy-phenoxy)-5- | 4-Chloro-2-methoxy- | 60 | 44 |
| | {4-[(4-chloro-phenyl)-methyl-amino]- | phenol | | |
| | benzoyl}-benzoic acid | | | |
| 6:6 | 2-(3-Chloro-2-fluoro-phenoxy)-5-{4- | 3-Chloro-2-fluoro-phenol | 67 | 77 |
| | [(4-chloro-phenyl)-methyl-amino]- | | | |
| | benzoyl}benzoic acid | | | |
| 6:7 | 5-{4-[(4-Chloro-phenyl)-methyl- | 2-Fluoro-3- | 90 | 84 |
| | amino]-benzoyl}-2-(2-fluoro-3- | trifluoromethyl-phenol | | |
| | trifluoromethyl-phenoxy)-benzoic acid | | | |
| 6:8 | 5-{4-[(4-Chloro-phenyl)-methyl- | 2-(1,1,2,2-Tetrafluoro- | 56 | 88 |
| | amino]-benzoyl}-2-[2-(1,1,2,2- | ethoxy)-phenol | | |
| | tetrafluoro-ethoxy)-phenoxy]-benzoic | | | |
| | acid | | | |
| 6:9 | 5-{4-[(4-Chloro-phenyl)-methyl- | 2,3-Dichloro-phenol | 68 | 74 |
| | amino]-benzoyl}-2-(2,3-dichloro- | | | |
| | phenoxy)-benzoic acid | | | |
| 6:10 | 5-{4-[(4-Chloro-phenyl)-methyl- | 5,6,7,8-Tetrahydro- | 72 | 89 |
| | amino]-benzoyl}-2-(5,6,7,8- | naphthalen-1-ol | | |
| | tetrahydro-naphthalen-1-yloxy)- | | | |
| | benzoic acid | | | |

TABLE 9
Inhibitors prepared via procedure J-M employing starting material XVII or
XVIII and subsequent hydrolysis according to method H

| | | Starting | | | |
|---|---|---|---|---|---|
| | | material/ | | Yield (%) | |
| No | Chemical name | method | Substrate | ester | acid |
| 7:1 | 2-(3-Chloro-benzoylamino)-5- | XVII/K | 3-Chloro-benzoyl | 67 | 77 |
| | [4-(3-chloro-benzoylamino)- | | chloride | | |
| | benzoyl]-benzoic acid | | | | |
| 7:2 | 2-(4-Chloro- | XVII/J | 4-Chloro- | 43 | 65 |
| | benzenesulfonylamino)-5-[4-(3- | | benzenesulfonyl | | |
| | chloro-benzoylamino)- | | chloride | | |
| | benzoyl]-benzoic acid | | | | |
| 7:3 | 5-[4-(3-Chloro-benzoylamino)- | XVII/J | 5-Chloro-2- | 52 | 43 |
| | benzoyl]-2-(5-chloro-2- | | methoxy- | | |
| | methoxy- | | benzenesulfonyl | | |
| | benzenesulfonylamino)- | | chloride | | |
| | benzoic acid | | | | |
| 7:4 | 5-[4-(3-Chloro-benzoylamino)- | XVII/K | 2,3-Difluoro- | 88 | 70 |
| | benzoyl]-2-(2,3-difluoro- | | benzoyl chloride | | |
| | benzoylamino)-benzoic acid | | | | |
| 7:5 | 2-[(Benzo[b]thiophene-7- | XVII/K | Benzo[b]thiophene- | 63 | 94 |
| | carbonyl)-amino]-5-[4-(3- | | 7-carbonyl | | |
| | chloro-benzoylamino)- | | chloride | | |
| | benzoyl]-benzoic acid | | | | |
| 7:6 | 5-[4-(3-Chloro-benzoylamino)- | XVII/K | 2,2-Difluoro- | 70 | 42 |
| | benzoyl]-2-[(2,2-difluoro- | | benzo[1,3]dioxole- | | |
| | benzo[1,3]dioxole-4-carbonyl)- | | 4-carbonyl) chloride | | |
| | amino]-benzoic acid | | | | |
| 7:7 | 5-[4-(3-Chloro-benzoylamino)- | XVII/K | 2-Chloro-6- | 54 | 64 |
| | benzoyl]-2-(3-chloro-2- | | (chlorocarbonyl)- | | |
| | hydroxy-benzoylamino)- | | phenyl acetate | | |
| | benzoic acid | | | | |
| 7:8 | 5-[4-(4-Chloro- | XVIII/K | 4-Chloro-6- | 41 | 74 |
| | benzenesulfonylamino)- | | (chlorocarbonyl)- | | |
| | benzoyl]-2-(5-chloro-2- | | phenyl acetate | | |
| | hydroxy-benzoylamino)- | | | | |
| | benzoic acid | | | | |
| 7:9 | 5-[4-(4-Chloro- | XVIII/K | 2-Fluoro-3- | 35 | 31 |
| | benzenesulfonylamino)- | | trifluoromethyl- | | |
| | benzoyl]-2-(2-fluoro-3- | | benzoyl chloride | | |
| | trifluoromethyl-benzoylamino)- | | | | |
| | benzoic acid | | | | |
| 7:10 | 5-[4-(4-Chloro- | XVIII/K | 2,3-Dichloro- | 40 | 46 |
| | benzenesulfonylamino)- | | benzoyl chloride | | |
| | benzoyl]-2-(2,3-dichloro- | | | | |
| | benzoylamino)-benzoic acid | | | | |
| 7:11 | 5-[4-(4-Chloro- | XVIII/K | 3-Chloro- | 17 | 51 |
| | benzenesulfonylamino)- | | benzoylamino | | |
| | benzoyl]-2-(3-chloro- | | chloride | | |
| | benzoylamino)-benzoic acid | | | | |

TABLE 10
Inhibitors prepared via procedure J-M employing starting material XIX
or XX and subsequent hydrolysis according to method H

| | | Starting | | Yield (%) | |
|---|---|---|---|---|---|
| No | Chemical name | material/method | Substrate | ester | acid |
| 8:1 | 2-(3,4-Difluoro-phenoxy)-5-[4- | XIX/L | 4-Bromo-1,2- | 77 | 96 |
| | (3,4-difluoro-phenylamino)- | | difluorobenzene | | |
| | benzoyl]-benzoic acid | | | | |
| 8:2 | 5-[4-(3-Chloro-phenylamino)- | XX/L | 3-Chloro-aniline | 74 | 52 |
| | benzoyl]-2-(3,4-difluoro- | | | | |
| | phenoxy)-benzoic acid | | | | |
| 8:3 | 5-[4-(4-Chloro-phenylamino)- | XX/L | 4-Chloro-aniline | 32 | 86 |
| | benzoyl]-2-(3,4-difluoro- | | | | |
| | phenoxy)-benzoic acid | | | | |
| 8:4 | 5-[4-(3-Chloro-benzoylamino)- | XX/Q | 3-Chloro- | 57 | 34 |
| | benzoyl]-2-(3,4-difluoro- | | benzoylamine | | |
| | phenoxy)-benzoic acid | | | | |
| 8:5 | 2-(3,4-Difluoro-phenoxy)-5-[4- | XXb/R | 4- | 25 | 97 |
| | (4-trifluoromethyl-phenoxy)- | | (Trifluoromethyl)- | | |
| | benzoyl]-benzoic acid | | phenol | | |

TABLE 11
Inhibitors prepared from hydrolysis of XXI according to method H

| | | | Yield (%) | |
|---|---|---|---|---|
| No | Chemical name | Substrate | ester | acid |
| 9:1 | 5-{4-[(4-Chloro-phenyl)-methyl-amino]- | 3-Trifluoromethyl- | 45 | 95 |
| | benzoyl}-2-(3-trifluoromethyl-phenylamino)- | phenylaniline | | |
| | benzoic acid | | | |
| 9:2 | 5-{4-[(4-Chloro-phenyl)-methyl-amino]- | 2-Trifluoromethyl- | 85 | 82 |
| | benzoyl}-2-(2-trifluoromethyl- | phenylaniline | | |
| | phenylamino)-benzoic acid | | | |
| 9:3 | 5-{4-[(4-Chloro-phenyl)-methyl-amino]- | 4-Trifluoromethyl- | 77 | 98 |
| | benzoyl}-2-(4-trifluoromethyl-phenylamino)- | phenylaniline | | |
| | benzoic acid | | | |

TABLE 12
Carboxylic acid isostere inhibitors

| | | | Yield (%) | |
|---|---|---|---|---|
| No | Chemical name | Substrate | Coupling | Cyclisation |
| 10:1 | {4-[(4-Chloro-phenyl)-methyl- | 5,6,7,8- | 40 | 23 |
| | amino]-phenyl}-[4-(5,6,7,8- | Tetrahydro- | | |
| | tetrahydro-naphthalen-1-yloxy)-3- | naphthalen-1- | | |
| | (1H-tetrazol-5-yl)-phenyl]- | ol | | |
| | methanone | | | |
| 10:2 | [4-(3-Chloro-phenoxy)-3-(1H- | 3-Chloro- | 89 | 63 |
| | tetrazol-5-yl)-phenyl]-{4-[(4-chloro- | phenol | | |
| | phenyl)-methyl-amino]-phenyl}- | | | |
| | methanone | | | |
| 10:3 | {4-[(4-Chloro-phenyl)-methyl- | 4- | 11 | 50 |
| | amino]-phenyl}-[3-(1H-tetrazol-5- | Trifluoromethyl- | | |
| | yl)-4-(4-trifluoromethyl- | phenylamine | | |
| | phenylamino)-phenyl]-methanone | | | |
| 10:4 | 2,3-Dichloro-N-[4-{4-[(4-chloro- | 2,3-Dichloro- | 21 | 22 |
| | phenyl)-methyl-amino]-benzoyl}-2- | benzoyl | | |
| | (1H-tetrazol-5-yl)-phenyl]- | chloride | | |
| | benzamide | | | |

TABLE 13
Di-acid inhibitors and subsequent hydrolysis according to method H

| | | | Yield (%) | |
|---|---|---|---|---|
| | | | es- | |
| No | Chemical name | Substrate | ter | acid |
| 11:1 | 5-{3-Carboxy-[4-(4-chloro- | 4-chloro- | 31 | 54 |
| | phenylamino)]-benzoyl}-2-(4- | phenylamine | | |
| | chloro-phenylamino)-benzoic acid | | | |
| 11:2 | 5-{3-Carboxy-4-[(4-chlorophenyl)- | 4-Chloro-N- | 75 | 44 |
| | methyl-amino]benzoyl}-2-[(4-chloro- | methylaniline | | |
| | phenyl)-methyl-amino]benzoic acid | | | |
| 11:3 | 5-{3-Carboxy-[4-(chlorophenoxy)]- | 4-Chlorophenol | 17 | 64 |
| | benzoyl}-2-(4-chlorophenoxy)- | | | |
| | benzoic acid | | | |

### Synthesis of Oxime Derivatives

### Example 12:1

### 2-(3-Chloro-benzoylamino)-5-{[4-(3-chloro-benzoylamino)-phenyl]-methoxyimino]-methyl}-benzoic Acid

2-(3-Chloro-benzoylamino)-5-[4-(3-chloro-benzoylamino)-benzoyl]-benzoic acid (Ex. 7:1) (0.20 g, 0.4 mmol) was dissolved in dry pyridine (10 mL) and O-methylhydroxylamine hydrochloride (0.07 g, 0.8 mmol) was added. The mixture was stirred at rt for a few days until no further conversion occurred. Concentration and addition of water formed a precipitate that was collected and washed with water. The residue was recrystallized to obtain the title compound as a mixture of the E and Z isomer (150 mg).

### Example 13:1

### 2-(3-Chloro-benzoylamino)-5-{[4-(3-chloro-benzoylamino)-phenyl]-hydroxyimino]-methyl}-benzoic Acid

### 2-(3-Chloro-benzoylamino)-5-[4-(3-chloro-benzoylamino)-benzoyl]-benzoic Acid (Ex. 7:1) (0.20 g, 0.4 mmol) was dissolved in dry pyridine (8 mL) and ethanol (20 mL). Hydroxylamine hydrochloride (0.06 g, 0.8 mmol) was added and the mixture was heated at rx for a few days until no further conversion occurred. Concentration and addition of water formed a precipitate that was collected and washed with water. The residue was purified by chromatography and recrystallized to obtain the title compound as a mixture of the E and Z isomer (140 mg).

TABLE 14
Spectroscopic Data of the Compounds of Examples 5:1 to 13:1

| No | ¹H NMR (DMSO-d₆, 400 or 200 MHz), δ: |
|---|---|
| 5:1 | 10.03 (1H, s) 9.29 (1H, s) 9.14 (1H, s) 8.32 (1H, d, J = 2.0 Hz) |
| | 7.81 (1H, dd, J = 8.8, 2.0 Hz) 7.74-7.57 (5H, m) 7.57-7.42 (2H, m) 7.34 (2H, |
| | m) 7.25-7.14 (2H, m) 7.09-6.97 (1H, m) |
| 5:2 | 13.6-13.3 (1H, br s) 10.03 (1H, s) 9.02 (1H, s) 8.60 (1H, s) 8.32 (1H, |
| | d, J = 2.0 Hz) 7.80 (1H, dd, J = 8.8, 2.0 Hz) 7.72-7.42 (6H, m) |
| | 7.41-7.29 (2H, m) 7.26-7.13 (2H, m) 6.93-6.78 (2H, m) 3.96 (2H, q, J = 7.3 Hz) |
| | 1.29 (3H, t, J = 7.3 Hz) |
| 5:3 | 11.8-10.8 (1H, br s) 8.90 (1H, s) 8.36 (1H, d, J = 2.4 Hz) 7.73-7.58 (3H, |
| | m) 7.49-7.27 (3H, m) 7.27-7.04 (5H, m) 7.04-6.94 (1H, m) |
| 5:4 | 13.8-13.0 (1H, br s) 12.0-11.3 (1H, br s) 10.64 (1H, s) 10.06 (1H, s) |
| | 8.34 (1H, d, J = 2.4 Hz) 7.95-7.79 (4H, m) 7.78-7.68 (2H, m) |
| | 7.58-7.38 (3H, m) 7.28-7.13 (2H, m) 7.02 (1H, d, J = 8.8 Hz) |
| 5:5 | 12.0-11.3 (1H, br s) 10.86 (1H, s) 8.40 (1H, d, J = 2.4 Hz) 7.87 (1H, d, |
| | J = 8.3 Hz) 7.95-7.81 (2H, m) 7.79-7.66 (3H, m) 7.65-7.27 (6H, m) |
| | 7.25-7.16 (1H, m) 7.15-7.02 (1H, m) |
| 5:6 | 14.1-12.5 (1H, br s) 10.05 (1H, s) 9.76 (1H, s) 8.32 (1H, d, J = 2.4 Hz) |
| | 8.24 (1H, d, J = 2.4 Hz) 7.91-7.59 (6H, m) 7.58-7.37 (2H, m) |
| | 7.29-7.11 (2H, m) 6.96 (1H, d, J = 8.8 Hz) |
| 5:7 | 14.0-12.6 (1H, br s) 10.2-9.9 (1H, br s) 9.07 (1H, s) 8.31 (1H, d, J = 2.1 Hz) |
| | 8.26 (1H, d, J = 2.9 Hz) 7.78 (1H, dd, J = 8.8, 2.1 Hz) 7.72-7.62 (3H, |
| | m) 7.55-7.37 (3H, m) 7.23-7.11 (4H, m) |
| 5:8 | 10.45 (1H, s) 10.05 (1H, s) 8.33 (1H, d, J = 2.0 Hz) 7.93-7.77 (3H, m) |
| | 7.77-7.66 (2H, m) 7.61 (1H, d, J = 8.3 Hz) 7.57-7.37 (2H, m) 7.27 (1H, |
| | d, J = 1.5 Hz) 7.25-7.04 (3H, m) 3.90 (3H, s) |
| 5:9 | 10.5-10.4 (1H, br s) 10.2-10.0 (1H, br s) 8.33 (1H, d, J = 2.4 Hz) |
| | 7.97-7.77 (3H, m) 7.77-7.66 (2H, m) 7.66-7.58 (1H, m) 7.57-7.35 (2H, m) |
| | 7.28 (1H, d, J = 2.0 Hz) 7.26-7.06 (3H, m) 3.91 (3H, s) |
| 5:10 | 12.4-11.9 (1H, br s) 10.93 (1H, s) 8.41 (1H, d, J = 2.4 Hz) |
| | 7.91-7.76 (3H, m) 7.76-7.56 (5H, m) 7.47-7.26 (2H, m) 7.20 (1H, d, J = 8.8 Hz) |
| | 7.14-6.96 (1H, m) |
| 5:11 | 13.8-13.0 (1H, br s) 10.65 (1H, s) 10.1 (1H, br s) 8.34 (1H, d, J = 2.4 Hz) |
| | 8.08-7.88 (4H, m) 7.83 (1H, dd, J = 8.8, 2.4 Hz) 7.79-7.64 (3H, m) |
| | 7.63-7.38 (3H, m) 7.29-7.11 (2H, m) |
| 5:12 | 12.2-11.8 (1H, br s) 11.2-11.0 (1H, br s) 8.64 (1H, d, J = 2.5 Hz) |
| | 8.45-8.37 (2H, m) 7.90-7.79 (2H, m) 7.77-7.65 (3H, m) 7.48-7.26 (2H, m) |
| | 7.20 (1H, d, J = 8.8 Hz) 7.12-7.00 (1H, m) |
| 6:1 | 13.7-12.5 (1H, br s) 8.08 (1H, d, J = 2.2 Hz) 7.82 (1H, dd, J = 8.4, 2.2 Hz) |
| | 7.69-7.60 (2H, m) 7.52-7.39 (4H, m) 7.34-7.24 (2H, m) |
| | 7.12-7.00 (3H, m) 6.91-6.81 (2H, m) 3.33 (3H, s, overlaped with water) |
| 6:2 | 8.08 (1H, d, J = 2.2 Hz) 7.83 (1H, dd, J = 8.4, 2.2 Hz) 7.71-7.61 (2H, m) |
| | 7.52-7.43 (2H, m) 7.38 (1H, d, J = 8.4 Hz) 7.34-7.27 (2H, m) |
| | 7.23-7.17 (1H, m) 7.16-7.08 (2H, m) 6.96 (1H, ddd, J = 8.4, 2.2, 0.8) |
| | 6.91-6.82 (2H, m) 3.33 (3H, s, overlaped with water) |
| 6:3 | 8.04 (1H, d, J = 2.1 Hz) 7.79 (1H, dd, J = 8.4 2.1) 7.70-7.60 (2H, m) |
| | 7.53-7.42 (3H, m) 7.38-7.17 (3H, m) 7.08 (1H, d, J = 8.4 Hz) |
| | 6.93-6.81 (3H, m) 3.33 (3H, s, overlaped with water) |
| 6:4 | 13.6-12.6 (1H, br s) 8.08 (1H, d, J = 2.2 Hz) 7.78 (1H, dd, J = 8.7, 2.2 Hz) |
| | 7.68-7.58 (3H, m) 7.51-7.43 (2H, m) 7.39 (1H, dd, J = 7.7, 1.8 Hz) |
| | 7.35-7.26 (3H, m) 7.21 (1H, dd, J = 7.7, 1.8 Hz) 7.11 (1H, dd, J = 7.7, |
| | 1.8 Hz) 6.91-6.82 (2H, m) 3.33 (3H, s, overlaped with water) |
| 6:5 | 13.5-12.7 (1H, br s) 8.04 (1H, d, J = 2.3 Hz) 7.72 (1H, dd, J = 8.7, 2.3 Hz) |
| | 7.67-7.57 (2H, m) 7.51-7.42 (2H, m) 7.36-7.25 (3H, m) 7.11 (1H, |
| | d, J = 8.7 Hz) 7.03 (1H, dd, J = 8.7, 2.3 Hz) 6.92-6.82 (2H, m) 6.73 (1H, |
| | d, J = 8.7 Hz) 3.76 (3H, s) 3.32 (3H, s, overlaped with water) |
| 6:6 | 13.4-13.1 (1H, br s) 8.11 (1H, d, J = 1.9 Hz) 7.84 (1H, dd, J = 8.6, 1.9) |
| | 7.71-7.59 (2H, m) 7.53-7.43 (2H, m) 7.42-7.36 (1H, m) 7.35-7.02 (5H, |
| | m) 6.92-6.80 (2H, m) 3.33 (3H, s, overlaped with water) |
| 6:7 | 8.11 (1H, d, J = 2.2 Hz) 7.85 (1H, dd, J = 8.5, 2.2) 7.71-7.60 (2H, m) |
| | 7.59-7.42 (3H, m) 7.41-7.26 (4H, m) 7.20 (1H, d, J = 8.5 Hz) |
| | 6.92-6.80 (2H, m) 3.33 (3H, s, overlaped with water) |
| 6:8 | 13.3-13.0 (1H, br s) 8.09 (1H, d, J = 1.8 Hz) 7.81 (1H, dd, J = 8.6, 1.8 Hz) |
| | 7.69-7.57 (2H, m) 7.52-7.42 (2H, m) 7.37 (1H, dd, J = 8.0, 1.5 Hz) |
| | 7.34-7.23 (3H, m) 7.15 (1H, dd, J = 8.0, 1.5 Hz) 6.98-6.80 (3H, m) |
| | 6.69 (1H, tt, J = 51.7, 3.0 Hz) 3.33 (3H, s, overlaped with water) |
| 6:9 | 13.4-12.9 (1H, br s) 8.12 (1H, dd, J = 2.1 Hz) 7.83 (1H, dd, J = 8.6, 2.1 Hz) |
| | 7.70-7.60 (2H, m) 7.52-7.43 (3H, m) 7.40-7.26 (3H, m) |
| | 7.08-6.98 (2H, m) 6.91-6.81 (2H, m) 3.33 (3H, s, overlaped with water) |
| 6:10 | 13.2-12.9 (1H, br s) 8.07 (1H, d, J = 2.1 Hz) 7.77 (1H, dd, J = 8.7, 2.1 Hz) |
| | 7.67-7.58 (2H, m) 7.51-7.42 (2H, m) 7.34-7.25 (2H, m) 7.13 (1H, |
| | t, J = 7.6 Hz) 6.95 (1H, d, J = 7.6 Hz) 6.90-6.82 (2H, m) 6.81-6.70 (2H, |
| | m) 3.32 (3H, s, overlaped with water) 2.83-2.67 (2H, m) 2.61-2.46 (2H, |
| | m, overlaped with DMSO) 1.77-1.60 (4H, m) |
| 7:1 | 12.50 (1H, s) 10.71 (1H, s) 8.83 (1H, d, J = 8.8 Hz) 8.42 (1H, d, J = 2.0 Hz) |
| | 8.12-7.90 (7H, m) 7.87-7.54 (6H, m) |
| 7:2 | 11.9-11.5 (1H, br s) 10.75-10.65 (1H, br s) 8.27 (1H, d, J = 2.0 Hz) |
| | 8.07-7.88 (7H, m) 7.80-7.53 (7H, m) |
| 7:3 | 12.2-11.6 (1H, br s) 10.8-10.5 (1H, br s) 8.27 (1H, d, J = 2.0 Hz) |
| | 8.04-7.85 (6H, m) 7.76-7.51 (6H, m) 7.23 (1H, d, J = 9.0 Hz) 3.82 (3H, s) |
| 7:4 | 12.28 (1H, s) 10.72 (1H, s) 8.85 (1H, d, J = 8.8 Hz) 8.41 (1H, d, J = 2.0 Hz) |
| | 8.13-7.91 (5H, m) 7.87-7.55 (6H, m) 7.51-7.37 (1H, m) |
| 7:5 | 12.79 (1H, s) 10.71 (1H, s) 8.95 (1H, d, J = 8.8 Hz) 8.44 (1H, d, J = 2.0 Hz) |
| | 8.23 (1H, d, J = 7.8 Hz) 8.14-7.91 (7H, m) 7.87-7.79 (2H, m) |
| | 7.74-7.55 (4H, m) |
| 7:6 | 14.7-13.4 (1H, br s) 12.34 (1H, s) 10.71 (1H, s) 8.87 (1H, d, J = 8.8 Hz) |
| | 8.42 (1H, d, J = 2.2 Hz) 8.13-7.90 (5H, m) 7.87-7.66 (5H, m) |
| | 7.65-7.54 (1H, m) 7.49-7.37 (1H, m) |
| 7:7 | 10.72 (1H, s) 8.72 (1H, d, J = 8.8 Hz) 8.41 (1H, d, J = 2.2 Hz) |
| | 8.11-7.90 (5H, m) 7.88-7.77 (3H, m) 7.76-7.66 (2H, m) 7.65-7.54 (1H, m) |
| | 7.14-7.02 (1H, m) |
| 7:8 | 14.0-13.2 (1H, br s) 12.8-12.2 (1H, br s) 12.0-11.4 (1H, br s) |
| | 11.1-10.7 (1H, br s) 8.84 (1H, d, J = 8.8 Hz) 8.32 (1H, d, J = 2.1 Hz) 7.99-7.81 (4H, |
| | m) 7.75-7.63 (4H, m) 7.49 (1H, dd, J = 2.7, 8.8 Hz) 7.33-7.23 (2H, m) |
| | 7.05 (1H, d, J = 8.8 Hz) |
| 7:9 | 12.4-12.2 (1H, br s) 11.1-11.0 (1H, br s) 8.78 (1H, d, J = 8.8 Hz) |
| | 8.32 (1H, d, J = 1.8 Hz) 8.30-8.18 (1H, m) 8.12-7.95 (2H, m) 7.91-7.80 (2H, |
| | m) 7.75-7.52 (5H, m) 7.33-7.22 (2H, m) |
| 7:10 | 11.95-11.80 (1H, br s) 11.1-11.0 (1H, br s) 8.68 (1H, d, J = 8.8 Hz) |
| | 8.30 (1H, d, J = 1.8 Hz) 7.99 (1H, dd, J = 8.8, 1.8 Hz) 7.92-7.80 (3H, m) |
| | 7.77-7.63 (5H, m) 7.60-7.49 (1H, m) 7.35-7.22 (2H, m) |
| 7:11 | 12.8-12.4 (1H, br s) 11.1-11.0 (1H, br s) 8.79 (1H, d, J = 8.8 Hz) |
| | 8.35 (1H, d, J = 1.2 Hz) 8.10-7.81 (5H, m) 7.80-7.60 (6H, m) 7.34-7.22 (2H, |
| | m) |
| 8:1 | 13.6-12.8 (1H, br s) 9.02 (1H, s) 8.11 (1H, d, J = 2.4 Hz) 7.85 (1H, dd, |
| | J = 8.3, 2.4 Hz) 7.78-7.63 (2H, m) 7.56-7.16 (4H, m) 7.16-7.06 (3H, m) |
| | 7.06-6.95 (1H, m) 6.95-6.82 (1H, m) |
| 8:2 | 9.07 (1H, s) 8.12 (1H, d, J = 2.0 Hz) 7.86 (1H, dd, J = 8.5, 2.0 Hz) |
| | 7.76-7.66 (2H, m) 7.56-7.39 (1H, m) 7.38-7.25 (2H, m) 7.24-7.07 (5H, m) |
| | 7.04-6.96 (1H, m) 6.94-6.83 (1H, m) |
| 8:3 | 9.02 (1H, s) 8.10 (1H, d, J = 2.2 Hz) 7.84 (1H, dd, J = 8.5, 2.2) |
| | 7.74-7.64 (2H, m) 7.55-7.31 (3H, m) 7.30-7.17 (3H, m) 7.15-7.06 (3H, m) |
| | 6.94-6.84 (1H, m) |
| 8:4 | 13.5-12.9 (1H, br s) 10.68 (1H, s) 8.16 (1H, d, J = 2.0 Hz) |
| | 8.04-7.95 (3H, m) 7.94-7.88 (2H, m) 7.82-7.78 (2H, m) 7.70-7.66 (1H, m) |
| | 7.58 (1H, t, J = 8.0 Hz) 7.48 (1H, m) 7.33-7.26 (1H, m) 7.13 (1H, d, J = 8.5 Hz) |
| | 6.94-6.88 (1H, m) |
| 8:5 | 13.8-13.0 (1H, br s) 8.15 (1H, d, J = 2.1 Hz) 7.98-7.75 (5H, m) |
| | 7.56-7.40 (1H, m) 7.40-7.17 (5H, m) 7.13 (1H, d, J = 8.7 Hz) 6.99-6.84 (1H, |
| | m) |
| 9:1 | 12.0-11.0 (1H, br s) 8.38 (1H, d, J = 2.1 Hz) 7.73 (1H, dd, J = 8.7, 2.1 Hz) |
| | 7.67-7.54 (5H, m) 7.52-7.36 (3H, m) 7.36-7.30 (2H, m) 7.28 (1H, |
| | d, J = 1.2 Hz) 7.01-6.84 (2H, m) 3.35 (3H, s) |
| 9:2 | 13.8-13.5 (1H, br s) 10.7-10.4 (1H, br s) 8.33 (1H, d, J = 2.1 Hz) |
| | 7.86-7.70 (4H, m) 7.68-7.59 (2H, m) 7.53-7.36 (3H, m) 7.36-7.24 (2H, m) |
| | 7.12 (1H, d, J = 8.7 Hz) 6.97-6.83 (2H, m) 3.35 (3H, s) |
| 9:3 | 12.1-11.5 (1H, br s) 8.38 (1H, d, J = 2.1 Hz) 7.73 (1H, dd, J = 8.7, 2.1 Hz) |
| | 7.70-7.57 (4H, m) 7.55-7.38 (5H, m) 7.35-7.26 (2H, m) |
| | 6.99-6.84 (2H, m) 3.35 (3H, s) |
| 10:1 | 8.79 (1H, d, J = 2.1 Hz) 7.85 (1H, dd, J = 8.7, 2.1 Hz) 7.79-7.69 (2H, m) |
| | 7.51-7.41 (2H, m) 7.37-7.27 (2H, m) 7.23 (1H, d, J = 8.7 Hz) |
| | 7.14-7.06 (1H, m) 7.04-6.97 (1H, m) 6.95-6.86 (2H, m) 6.80 (1H, d, J = 8.7 Hz) |
| | 3.41 (3H, s) 2.89-2.76 (2H, m) 2.63-2.51 (2H, m) 1.82-1.64 (4H, m) |
| 10:2 | 17.0-15.9 (1H, br s) 8.42 (1H, d J = 2.1 Hz) 7.87 (1H, dd, J = 8.7, 2.1 Hz) |
| | 7.76-7.64 (2H, m) 7.58-7.44 (3H, m) 7.44-7.28 (4H, m) 7.27-7.19 (1H, |
| | m) 7.09 (1H, d, J = 8.7 Hz) 6.95-6.83 (2H, m) 3.36 (3H, s, overlapped |
| | with DMSO) |
| 10:3 | 11.55-11.45 (1H, br s) 8.62 (1H, d J = 1.8 Hz) 7.75-7.57 (6H, m) |
| | 7.56-7.43 (4H, m) 7.37-7.28 (2H, m) 6.98-6.88 (2H, m) 3.37 (3H, s) |
| 10:4 | 8.88 (1H, d, J = 8.7 Hz) 8.62 (1H, d, J = 1.8 Hz) 7.87-7.63 (6H, m) |
| | 7.52-7.38 (3H, m) 7.31-7.23 (2H, m) 6.94-6.85 (2H, m) 3.40 (3H, s) |
| 11:1 | 13.7-13.2 (2H, br s) 10.10 (2H, s) 8.33 (2H, d, J = 2.1 Hz) 7.79 (2H, dd, |
| | J = 8.7, 2.1 Hz) 7.56-7.31 (8H, m) 7.22 (2H, d, J = 8.7 Hz) |
| 11:2 | 8.12-7.94 (2H, m) 7.92-7.77 (2H, m) 7.39 (2H, d, J = 8.4 Hz) |
| | 7.23-7.11 (4H, m) 6.79-6.64 (4H, m) 3.25 (6H, s) |
| 11:3 | 13.4-13.1 (2H, br s) 8.20 (2H, d, J = 2.1 Hz) 7.93 (2H, dd, J = 8.7, 2.1 Hz) |
| | 7.53-7.42 (4H, m) 7.16-7.07 (6H, m) |
| 12:1 | 12.3-12.2 (1H, br s) 10.6-10.5 (1H, br s) 8.73 and 8.66 (1H, d, J = 8.7 Hz |
| | and d, J = 8.7 Hz) 8.07-7.76 (7H, m) 7.75-7.29 (7H, m) 3.91 (3H, s) |
| 13:1 | 15.7-15.4 (1H, br s) 11.23 and 11.19 (1H, s and s) 10.59 and |
| | 10.56 (1H, s and s) 8.71 and 8.64 (1H, d, J = 8.7 Hz and d, J = 8.7 Hz) |
| | 8.16-7.75 (7H, m) 7.72-7.45 (5H, m) 7.43-7.29 (2H, m) mixture of E/Z |
| | isomers |

### Synthesis of Inhibitors in Table 15

### Preparation of methyl 5-(4-aminobenzoyl)-2-(2,4-dichlorobenzamido)benzoate

Step 1: Methyl 5-(4-(((9H-fluoren-9-yl)methoxy)carbonylamino)benzoyl)-2-aminobenzoate was synthesized according to the preparation of XVII using a standard Fmoc protection of methyl 5-(4-aminobenzoyl)-2-fluorobenzoate in step 1 (Fmoc chloride and pyridine in dichloromethane at 0° C.). Step 1 was repeated again after step 2.

Step 2: Aroylation of methyl 5-(4-(((9H-fluoren-9-yl)methoxy)carbonyl-amino)benzoyl)-2-aminobenzoate according to method N using 2,4-dichlorobenzoyl chloride furnished methyl 5-(4-(((9H-fluoren-9-yl)methoxy)carbonylamino)benzoyl)-2-(2,4-dichlorobenzamido)benzoate (58%).

Step 3: Methyl 5-(4-(((9H-fluoren-9-yl)methoxy)carbonylamino)benzoyl)-2-(2,4-dichlorobenzamido)benzoate (0.9 g, 1.35 mmol) and piperidine (0.946 g, 11.11 mmol) were mixed in dry DMF at rt for 1 h. Extractive workup (EtOAc, water, brine) with drying (Na₂SO₄) and concentration of the organic extracts furnished, after purification by chromatography, pure methyl 5-(4-aminobenzoyl)-2-(2,4-dichlorobenzamido)benzoate 0.36 g, 60%).

Examples 14:1 and 14:2 was prepared by reacting methyl 5-(4-aminobenzoyl)-2-(2,4-dichlorobenzamido)benzoate with the corresponding acid chloride (see table 14) according to method N using pyridine (Ex. 14:1) and toluene (Ex. 14:2) as solvent. Final hydrolysis according to procedure H furnished the inhibitors depicted in table 15.

Example 14:3 was prepared by reacting methyl 5-(4-((4-chlorophenyl)-(methyl)amino)benzoyl)-2-(trifluoromethylsulfonyloxy)benzoate (see preparation of XXI) with 4-tert-butylcyclohexanamine, according to the preparation of XII, followed by hydrolysis according to procedure H to furnish the inhibitor depicted in table 15.

TABLE 15

| | | | Yield (%) | |
|---|---|---|---|---|
| No | Chemical name | Substrate | ester | acid |
| 14:1 | 5-[4-(2-Cyclopentyl-acetylamino)-benzoyl]- | 2-cyclopentylacetyl | 70 | 89 |
| | 2-(2,4-dichloro-benzoylamino)-benzoic acid | chloride | | |
| 14:2 | 2-(2,4-Dichloro-benzoylamino)-5-(4- | heptanoyl chloride | 42 | 80 |
| | heptanoylamino-benzoyl)-benzoic acid | | | |
| 14:3 | 2-(4-tert-Butyl-cyclohexylamino)-5-{4-[(4- | 4-tert-butylcyclo- | 27 | 98 |
| | chloro-phenyl)-methyl-amino]-benzoyl}- | hexanamine | | |
| | benzoic acid, mixture of stereoisomers | | | |

TABLE 16
Spectroscopic Data of the Compounds of Table 15

| No | ¹H NMR (DMSO-d₆, 400 or 200 MHz), δ: |
|---|---|
| 14:1 | 11.92 (1H, s) 10.26 (1H, s) 8.73 (1H, d, J = 8.7 Hz) 8.36 (1H, d, J = 2.0 Hz) |
| | 8.04 (1H, dd, J = 8.7 and 2.0 Hz) 7.89-7.70 (6H, m) 7.67-7.60 (1H, dd, J = 8.4 |
| | 2.0 Hz) 2.39-2.14 (3H, m) 1.86-1.43 (6H, m) 1.35-1.09 (2H, m) |
| 14:2 | 11.92 (1H, s) 10.28 (1H, s) 8.73 (1H, d, J = 8.7 Hz) 8.36 (1H, d, J = 2.0 Hz) |
| | 8.04 (1H, dd, J = 8.7 and 2.0 Hz) 7.87-7.57 (7H, m) 2.36 (2H, t, J = 7.3 Hz) |
| | 1.70-1.49 (2H, m) 1.38-1.16 (6H, m) 0.94-0.79 (3H, m) |
| 14:3 | 13.3-12.6 (1H, br s) 9.0-8.8 (0.5H, m) 8.4-8.3 (0.5H, m) 8.21 (1H, dd, J = 7.4, |
| | 2.0 Hz) 7.80-7.64 (1H, m) 7.62-7.48 (2H, m) 7.48-7.35 (2H, m) |
| | 7.31-7.20 (2H, m) 6.95-6.76 (3H, m) 3.96-3.86 (0.5H, m) 3.42-3.37 (0.5H, m) 3.30 (3H, |
| | s) 2.10-2.03 (1H, m) 1.88-1.71 (2H, m) 1.65-1.44 (2H, m) 1.25-1.08 (4H, m) |
| | 0.83 and 0.82 (9H, two s) (a mixture of stereoisomers) |

### Example 20

Title compounds of the examples were tested in the biological test described above and were found to exhibit the following percentage inhibitions of LTC₄ at a concentration of 10 μM. For example, the following representative compounds of the examples exhibited the percentage inhibitions:

| | Percantage Inhibition at 10 μM |
|---|---|
| | (unless specified |
| Ex. | otherwise) |
| 1 | 93 |
| 2 | 99 (exhibitied an IC₅₀ of 258 nM) |
| 3 | 98 |
| 4 | 100 (exhibitied an IC₅₀ of 191 nM) |
| 5 | 98 |
| 6 | 95 |
| 7 | 97 |
| 8 | 100 (exhibitied an IC₅₀ of 73 nM) |
| 9 | 92 (at a concentration of 0.3 μM) |
| 10 | 82 |
| 11 | 100 |
| 12 | 98 |
| 13 | 100 (exhibitied an IC₅₀ of 86 nM) |
| 14 | 100 |
| 15 | 68 |
| 16 | 94 |
| 17 | 99 |
| 18 | 100 |
| 19 | 75 |
| 5:1 | 95 |
| 5:2 | 100 |
| 5:3 | 84 |
| 5:4 | 78 |
| 5:5 | 97 |
| 5:6 | 84 |
| 5:7 | 90 |
| 5:10 | 98 |
| 5:11 | 87 |
| 5:12 | 98 |
| 6:1 | 62 |
| 6:2 | 96 |
| 6:3 | 90 |
| 6:4 | 97 |
| 6:5 | 98 |
| 6:6 | 83 |
| 6:7 | 96 |
| 6:8 | 98 |
| 6:9 | 66 |
| 6:10 | 88 |
| 7:1 | 97 |
| 7:2 | 100 |
| 7:3 | 96 |
| 7:4 | 100 |
| 7:5 | 100 |
| 7:6 | 100 |
| 7:7 | 82 |
| 7:8 | 96 |
| 7:9 | 99 |
| 7:10 | 99 |
| 7:11 | 98 |
| 8:1 | 97 |
| 8:2 | 98 |
| 8:3 | 100 |
| 8:4 | 88 |
| 8:5 | 96 |
| 9:1 | 90 |
| 9:2 | 55 |
| 9:3 | 86 |
| 10:1 | 97 |
| 10:2 | 84 |
| 10:3 | 71 |
| 10:4 | 77 |
| 11:1 | 97 |
| 11:2 | 82 |
| 11:3 | 97 |
| 14:1 | 93 |
| 14:2 | 100 |
| 14:3 | 86 |

1. A compound of formula I, 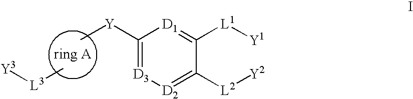
wherein
Y represents —C(O)— or —C(═N—OR²⁸)—;
R²⁸ represents hydrogen or C₁₋₆ alkyl optionally substituted by one or more halo atoms;
each of D₁, D₂ and D₃ respectively represent —C(R^{1a})═, —C(R^{1b})═ and —C(R^{1c})═, or,
each of D₁, D₂ and D₃ may alternatively and independently represent —N═;
ring A represents: 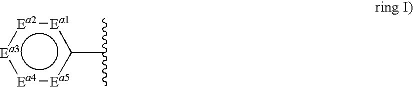
each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{a1} E^{a2}, E^{a3}, E^{a4} and E^{a5} may alternatively and independently represent —N═;
one of R^{2b}, R^{2c} and R^{2d} represents the requisite -L³-Y³ group, and the others independently represent hydrogen, -L^{1a}-Y^{1a} or a substituent selected from X¹; 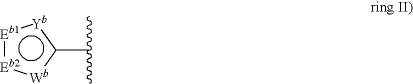
E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or 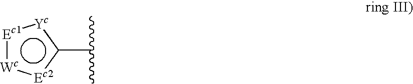
E^{c1} and E^{c2} each respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c}, R^{4b} and, if present, R^{4c} and R^{4d} represents the requisite -L³-Y³ group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c}, independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5b}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —NO₂, N(R^{5g})S(O)₂N(R^{6f})R^{7f}, —OR^{5b}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{1a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O, —OR^{5b} and R^{5a};
R⁵ⁱ, R^{5m} and R^{5p} independently represent R^{5a};
R^{5a} represents C₁₋₆ alkyl optionally substituted by one or more substituents selected from halo, —ON, —N₃, ═O, —OR^{8a}, —N(R^{8b})R^{8c}, —S(O)ₙR^{8d}, —S(O)₂N(R^{8e})R^{8f} and —OS(O)₂N(R^{8g})R^{8h};
n represents 0, 1 or 2;
R^{8a}, R^{8b}, R^{8d}, and R^{8g} independently represent H or C₁₋₆ alkyl optionally substituted by one or more substituents selected from halo, ═O, —OR^{11a}, —N(R^{12a})R^{12b} and —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and —S(O)₂-M²; or R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O and C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R¹², R^{12b}, R^{14a}, R^{14b}, R^{15a} and R^{15b} independently represent H, —CH₃ or —CH₂CH₃, Y¹ and Y^{1a} independently represent, on each occasion when used herein, —N(H)SO₂R^{9a}, —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9b})₂, —C(CF₃)₂OH, —S(O)₂N(R¹⁰ⁱ)R⁹ⁱ or any one of the following groups: 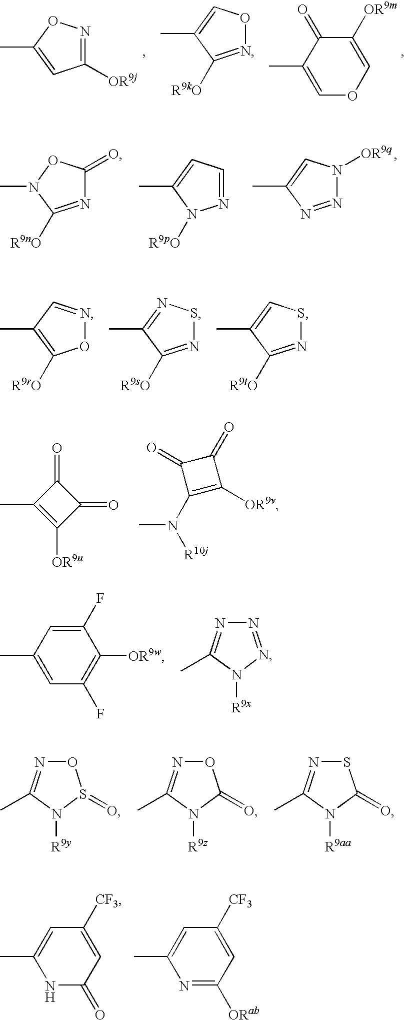
R^{9a} to R^{9z}, R^{9aa}, R^{9ab}, R^{10f}, R^{10g}, R¹⁰ⁱ and R^{10j} independently represent C₁₋₈ alkyl or a heterocycloalkyl group, both of which are optionally substituted by one or more substituents selected from G¹ and/or Z¹; or
R^{9b} to R^{9z}, R^{9aa}, R^{9ab}, R^{10f}, R^{10g}, R¹⁰ⁱ and R^{10j} independently represent hydrogen; or
R^{9a} to R^{9z}, R^{9aa}, R^{10f}, R^{10g}, R¹⁰ⁱ and R^{10j} independently represent C₁₋₈ alkyl or a heterocycloalkyl group, both of which are optionally substituted by one or more substituents selected from G¹ and/or Z¹; or
R^{9b} to R^{9z}, R^{9aa}, R^{10f}, R^{10g}, R¹⁰ⁱ and R^{10j} independently represent hydrogen; or
any pair of R^{9f} and R^{10f}, R^{9g} and R^{10g}, and R⁹ⁱ and R¹⁰ⁱ, may be linked together to form, along with the atom(s) to which they are attached, a 3- to 6-membered ring, which ring optionally contains a further heteroatom, in addition to the nitrogen atom to which these substituents are necessarily attached, and which ring is optionally substituted by one or more substituents selected from F, Cl, ═O, —OR^{5h} and R^{5a};
one of Y² and Y³ represents an aryl group or a heteroaryl group (both of which groups are optionally substituted by one or more substituents selected from A) and the other represents either:
(a) an aryl group or a heteroaryl group (both of which groups are optionally substituted by one or more substituents selected from A); or
(b) C₁₋₁₂ alkyl optionally substituted by one or more substituents selected from G¹ and/or Z¹;
but wherein:
(a) when Y² represents C₁₋₁₂ alkyl, then it is C₁₋₆ alkyl optionally substituted b one or more G¹ substituents; and
(b) when Y³ resents C₁₋₁₂ alkyl, then it is a cyclic C₃₋₆ alkyl group optionally substituted by one or more G¹ substituents;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{16a};
wherein A¹ represents a single bond or a spacer group selected from —C(O)A²-, —S—, —S(O)₂A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents ═O, ═S, ═NOR^{16b}, ═NS(O)₂N(R^{17f})R^{16c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{18a};
wherein A⁶ represents a single bond or a spacer group selected from —C(O)A⁷-, —S—, —S(O)₂A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{20a};
wherein A¹¹ represents a single bond or a spacer group selected from —C(O)A¹²-, —S—, —S(O)₂A¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents ═O, ═S, ═NOR^{20b}, ═NS(O)₂N(R^{21f})R^{20c}, ═NCN or ═C(H)NO₂;
R^{20a}, R^{20b}; R^{20c}; R^{21a}, R^{21b}; R^{21c}, R^{21d}; R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ and L^{1a} independently represent a single bond or —(CH₂)ₚ-Q-(CH₂)_{q}—;
Q represents —C(R^{y1})(R^{y2})—, —C(O)— or —O—;
R^{y1} and R^{y2} independently represent H, F or X⁴; or
R^{y1} and R^{y2} may be linked together to form a 3- to 6-membered ring, which ring optionally contains a heteroatom, and which ring is optionally substituted by one or more substituents selected from F, Cl, ═O and X⁵;
L² and L³ independently represent a single bond or a spacer group selected from —(CH₂)ₚ—C(R^{y3})(R^{y4})—(CH₂)_{q}-A¹⁶, —C(O)A¹⁷, —S—, —SC(R^{y3})(R^{y4})—, —S(O)₂A¹⁸-, —N(R^{w})A¹⁹- or —OA²⁰-, in which:
A¹⁶ represents a single bond, —O—, —N(R^{w})—, —C(O)—, or —S(O)ₘ—;
A¹⁷ and A¹⁸ independently represent a single bond, —C(R^{y3})(R^{y4})—, —O—, or —N(R^{w});
A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;
p and q independently represent 0, 1 or 2;
m represents 0, 1 or 2;
R^{y3} and R^{y4} independently represent H, F or X⁶; or
R^{y3} and R^{y4} may be linked together to form a 3- to 6-membered ring, which ring optionally contains a heteroatom, and which ring is optionally substituted by one or more substituents selected from F, Cl, ═O and X⁷;
R^{w} represents H or X⁸;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, —CN, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, —CN, C₁₋₄ alkyl (optionally substituted by one or more substituents selected from fluoro, chloro and ═O), —N(R^{26a})R^{26b} and —OR^{28c});
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt or prodrug thereof,
provided that:
when D₁, D₂ and D₃ all represent —C(H)═; ring A represents ring (I); E^{a1}, E^{a2}, E^{a3}, E^{a4} and E^{a5} respectively represent —C(H)═, —C(R^{2b})═, —C(R^{2c})═, —C(R^{2d})═ and —C(H)═; R^{2d} represents H; L¹ and L^{1a} both represent single bonds; Y¹ and Y^{1a} both represent —C(O)OR^{9b}; R^{9b} represents H:
(A) R^{2c} represents -L³-Y³; R^{2b} represents -L^{1a}-Y^{1a}; L² and L³ both represent —N(R^{w})A¹⁹-; R^{w} represents H; A¹⁹ represents —C(O)—, then Y² and Y³ do not both represent 1-naphthyl;
(B) L² and L³ both represent —C(O)A¹⁷-, A¹⁷ represents —N(R^{w})—; R^{w} represents H:
(i) R^{2b} represents -L³-Y³; R^{2c} represents -L^{1a}-Y^{1a}, then:
(I) Y² and Y³ do not both represent 4-pyridyl, 2-pyridyl, 4-methylphenyl or 4-methoxyphenyl;
(II) Y² and Y³ do not both represent phenyl substituted in the meta-position by a G¹ substituent in which G¹ is chloro, and in the para-position by methyl substituted by G¹, in which G¹ represents -A¹-R^{16a}; A¹ represents a single bond, and R^{16a} represents a heterocycloalkyl group that is 2-isoxazolidinyl group substituted in the 3-position with a Z³ group that is ═O and at the 4-position with two G³ groups in which G³ represents -A¹¹-R^{20a}, A¹¹ is a single bond; and R^{20a} represents —CH₃;
(ii) R^{2c} represents -L³-Y³; R^{2b} represents Y^{1a}, then:
(I) Y² and Y³ do not both represent 4-bromophenyl, phenyl, 4-methylphenyl, 4-methoxyphenyl, 3-nitro-4-aminophenyl or 3-nitro-4-hydroxy-phenyl, or, one of Y² or Y³ does not represent 4-bromophenyl when the other represents unsubstituted phenyl;
(II) when Y² and Y³ both represent phenyl substituted by A:
(1) A represents G¹; G¹ represents -A¹-R^{16a}: R^{16a} represents phenyl substituted by G³; G³ represents -A¹¹-R^{20a}; -A¹¹ represents N(R^{21a})A¹⁴; A¹⁴ represents —C(O)—; R^{21a} represents H; and R^{20a} represents an alkyl group terminally substituted at the same carbon atom with both a ═O and a —OR^{22b} group, in which R^{22b} is hydrogen when:
(a) A and G³ are both in the para-position, and R^{20a} represents either a C₄ alkyl group that is —CH═C(CH₃)₂ or a C₃ alkyl group that is —C(H)═C(H)—CH₃ (both of which are terminally substituted at one of the CH₃ groups), then when A¹ represents —OA⁵-, then A⁵ does not represent a single bond;
(b) A and G³ are both in the para-position, and R^{20a} represents —CH═C(CH₃)₂ (terminally substituted at one of the CH₃ groups), then when A¹ represents —S(O)₂A³, then A³ does not represent a single bond;
(c) A and G³ are both in the meta-position, and R^{20a} represents a —C(H)═C(H)—CH₃ (terminally substituted at the CH₃ group), then when A¹ represents —S(O)₂A³, then A³ does not represent a single bond;
(2) A represents methyl substituted by G¹; G¹ represents -A¹-R^{16a}, A¹ represents a single bond, R^{16a} phenyl substituted in the para-position by G³; G³ represents -A¹¹-R^{20a}; -A¹¹ represents
—N(R^{21a})A¹⁴; A¹⁴ represents —C(O)—; R^{21a} represents H; and R^{20a} represents either a C₄ alkyl group that is —CH₂—C(═CH₂)—CH₃ or a C₃ alkyl group that is —C(H)═C(H)—CH₃, then the latter two alkyl groups are not both terminally substituted at the respective —CH₃ moieties with both a ═O and a —OR^{22b} group, in which R^{22b} is hydrogen.

2-36. (canceled)

37. The compound of claim 1, wherein:
one of Y² and Y³ represents aryl or heteroaryl (both of which are optionally substituted by one or more substituents selected from A) and the other represents either:
(a) aryl or heteroaryl (both of which are optionally substituted by one or more substituents selected from A); or
(b) a cyclic C₃₋₆ alkyl group optionally substituted by one or more G¹ substituents.

38. The compound of claim 37, wherein Y² and Y³ independently represent aryl or heteroaryl, both of which are optionally substituted by one or more substituents selected from A.

39. The compound of claim 1, wherein L³ (and/or L²) represents —OA²⁰-, —S—, —SC(R^{y3})(R^{y4})—, —(CH₂)ₚ—C(R^{y3})(R^{y4})—(CH₂)_{q}-A¹⁶-, —S(O)₂A¹⁸- or —N(R^{w})A¹⁹-.

40. The compound of claim 1, wherein D₁, D₂ and D₃ independently represent —C(H)═.

41. The compound of claim 1, wherein ring A represents ring (I).

42. The compound of claim 1, wherein E^{a1} and E^{a5} independently represent —C(H)═ and E^{a2}, E^{a3} and E^{a4} respectively represent —C(R^{2b})═, —C(R^{2c})═ and —C(R^{2d})═.

43. The compound of claim 1, wherein R^{2b} represents H or -L^{1a}-Y^{1a}.

44. The compound of claim 1, wherein R^{2c} represents the requisite -L³-Y³ group.

45. The compound of claim 1, wherein R^{2d} represents H.

46. The compound of claim 1, wherein L¹ and L^{1a} independently represent a single bond.

47. The compound of claim 1, wherein Y¹ and Y^{1a} independently represent —C(O)OR^{9b}.

48. The compound of claim 1, wherein R^{9b} represents C₁₋₆ alkyl or H.

49. The compound of claim 1, wherein L² and L³ independently represent —N(R^{w})A¹⁹-.

50. The compound of claim 1, wherein A¹⁹ represents a single bond, —S(O)₂—, —C(O)— or —C(O)N(R^{w})—.

51. The compound of claim 1, wherein R^{w} represents C₁₋₃ alkyl or H.

52. The compound of claim 1, wherein Y² and Y³ independently represent optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl.

53. The compound of claim 52, wherein Y² and Y³ independently represent optionally substituted naphthyl, 2-benzoxazolyl, 2-benzimidazolyl, 2-benzothiazolyl, thienyl, oxazolyl, thiazolyl, pyridyl or phenyl.

54. The compound of claim 53, wherein Y² and Y³ independently represent phenyl optionally substituted by one or more substituents selected from A.

55. The compound of claim 52, wherein the optional substituents are selected from halo; cyano; —NO₂; C₁₋₆ alkyl optionally substituted with one or more halo groups; heterocycloalkyl optionally substituted by one or more substituents selected from C₁₋₃ alkyl and ═O; —OR²⁶; —C(O)R²⁶; —C(O)OR²⁶; and —N(R²⁶)R²⁷; wherein R²⁶ and R²⁷ independently represent H, C₁₋₆ alkyl (optionally substituted by one or more halo groups) or aryl (optionally substituted by one or more halo or C₁₋₃ alkyl groups (which alkyl group is optionally substituted by one or more halo atoms)).

56. The compound of claim 1, wherein A represents G¹ or C₁₋₆ alkyl optionally substituted by one or more substituents selected from G¹.

57. The compound of claim 1, wherein G¹ represents halo, NO₂ or -A¹-R^{16a}.

58. The compound of claim 1, wherein A¹ represents —O—.

59. The compound of claim 1, wherein R^{16a} represents hydrogen or C₁₋₆ alkyl optionally substituted by one or more substituents selected from G³.

60. The compound of claim 1, wherein G³ represents halo.

61. The compound of claim 1, but without proviso (B), or a pharmaceutically acceptable salt thereof, for use as a pharmaceutical.

62. The pharmaceutical formulation comprising a compound of claim 1, but without proviso (B), or a pharmaceutically acceptable salt thereof, in admixture with a pharmaceutically acceptable adjuvant, diluent or carrier.

63. The compound of claim 1, but without the provisos, or a pharmaceutically acceptable salt thereof, for use in the treatment of a disease in which inhibition of the synthesis of leukotriene C₄ is desired and/or required.

64. A use of a compound as defined in claim 1, but without the provisos, or a pharmaceutically acceptable salt thereof, for the manufacture of a medicament for the treatment of a disease in which inhibition of the synthesis of leukotriene C₄ is desired and/or required.

65. The compound of claim 63 or use of claim 64, wherein the disease is a respiratory disease, inflammation and/or has an inflammatory component.

66. The compound or use of claim 65 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy.

67. The compound or use of claim 66, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

68. A method of treatment of a disease in which inhibition of the synthesis of leukotriene C₄ is desired and/or required, which method comprises administration of a therapeutically effective amount of a compound as defined in claim 1, but without the provisos, or a pharmaceutically-acceptable salt thereof, to a patient suffering from, or susceptible to, such a condition.

69. A combination product comprising:
(A) a compound as defined in claim 1, but without the provisos, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

70. The combination product of claim 69 which comprises a pharmaceutical formulation including a compound as defined in claim 1 but without the provisos, or a pharmaceutically-acceptable salt thereof, another therapeutic agent that is useful in the treatment of a respiratory disorder and/or inflammation, and a pharmaceutically-acceptable adjuvant, diluent or carrier.

71. The combination product of claim 69 which comprises a kit of parts comprising components:
(a) a pharmaceutical formulation including a compound as defined in claim 1 but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier, which components (a) and (b) are each provided in a form that is suitable for administration in conjunction with the other.

72. A process for the preparation of a compound as defined in claim 1, which process comprises:
(i) for compounds of formula I in which Y represents —C(O)—, oxidation of a compound of formula II, 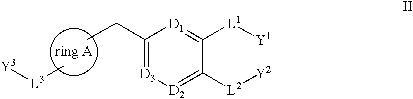
wherein ring A, D₁, D₂, D₃, L¹, Y¹, L², Y², L³ and Y³ are as defined in claim 1;
(ii) for compounds of formula I in which L² and/or L³ represents —N(R^{w})A¹⁹- in which R^{w} represents H, reaction of a compound of formula III, 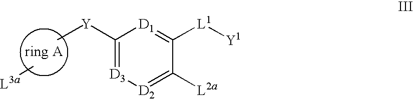
or a protected derivative thereof wherein L^{2a} represents —NH₂ or —N(R^{w})A¹⁹-Y², L^{3a} represents —NH₂ or —N(R^{w})A¹⁹-Y³, provided that at least one of L^{2a} and L^{3a} represents —NH₂, and Y, ring A, D₁, D₂, D₃, L¹ and Y¹ are as defined in claim 1, with:
(A) when A¹⁹ represents —C(O)N(R^{w})—, in which R^{w} represents H:
(a) a compound of formula IV,
Y^{a}—N═C═O IV
; or
(b) with CO (or a reagent that is a suitable source of CO), phosgene or triphosgene in the presence of a compound of formula V,
Y^{a}—NH₂ V
wherein, in both cases, Y^{a} represents Y² or Y³ (as appropriate/required) as defined in claim 1;
(B) when A¹⁹ represents —S(O)₂N(R^{w})—:
(a) ClSO₃H, PCl₅₃ and then a compound of formula V as defined above;
(b) SO₂Cl₂, and then a compound of formula V as hereinbefore defined;
(c) a compound of formula VA,
Y^{a}—N(H)SO₂Cl VA
wherein Y^{a} is as defined above;
(d) ClSO₂N═C═O, and then a compound of formula V as defined above;
(C) when A¹⁹ represents a single bond, with a compound of formula VI,
Y^{a}-L^{a} VI
wherein L^{a} represents a suitable leaving group and Y^{a} is as defined above;
(D) when A¹⁹ represents —S(O)₂—, —C(O)—, —C(R^{y3})(R^{y4})—, —C(O)—C(R^{y3})(R^{y4})— or —C(O)O—, with a compound of formula VII,
Y^{a}-A^{19a}-L^{a} VI
wherein A^{19a} represents —S(O)₂—, —C(O)—, —C(R^{y3})(R^{y4})—, —C(O)—C(R^{y3})(R^{y4})— or —C(O)O—, and Y^{a} and L^{a} are as defined above;
(iii) for compounds of formula I in which one of L² and L³ represents —N(R^{w})C(O)N(R^{w})— and the other represents —NH₂ (or a protected derivative thereof) or —N(R^{w})C(O)N(R^{w})—, in which R^{w} represents H (in all cases) reaction of a compound of formula VIII, 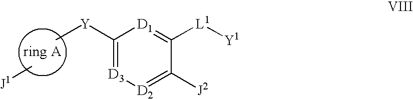
wherein one of J¹ or J² represents —N═C═O and the other represents —NH₂ (or a protected derivative thereof) or —N═C═O (as appropriate), and Y, ring A, D₁, D₂, D₃, L¹ and Y¹ are as defined in claim 1, with a compound of formula V as defined above;
(iv) for compounds of formula I in which L² and L³ independently represent a single bond, —S—, —SC(R^{y3})(R^{y4})—, —N(R^{w})A¹⁹- or —OA²⁰-, reaction of a compound of formula IX, 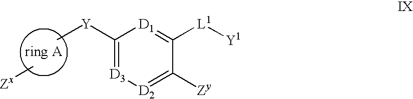
wherein at least one of Z^{X} and Z^{y} represents a suitable leaving group and the other may also independently represent a suitable leaving group, or, Z^{y} may represent -L²-Y² and Z^{X} may represent -L³-Y³, and Y, ring A, D₁, D₂, D₃, L¹, Y¹, L², s Y L³ and Y³ are as defined in claim 1, with a (or two separate) compound(s) (as appropriate/required) of formula X,
Y^{a}-L^{x}-H X
wherein L^{x} represents a single bond, —S—, —SC(R^{y3})(R^{y4})—, —N(R^{w})A¹⁹- or —OA²⁰-, and Y^{a} is as defined above;
(v) compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), may be prepared by reaction of a corresponding compound of formula I in which such a group is present that does represent hydrogen with a compound of formula X¹,
R^{wy}-L^{b} XI
wherein R^{wy} represents either R^{w} (as appropriate) as defined in claim 1 provided that it does not represent hydrogen (or R^{w} represents a R⁵ to R¹⁹ group in which those groups do not represent hydrogen), and L^{b} represents a suitable leaving group;
(vi) for compounds of formula I that contain only saturated alkyl groups, reduction of a corresponding compound of formula I that contains an unsaturation;
(vii) for compounds of formula I in which Y¹ and/or, if present, Y^{1a} represents —C(O)OR^{9b}, —S(O)₃R^{9c}, —P(O)(OR^{9d})₂, or —B(OR^{9h})₂, in which R^{9b}, R^{9c}, R^{9d} and R^{9h} represent hydrogen, hydrolysis of a corresponding compound of formula I in which R^{9b}, R^{9c}, R^{9d} or R^{9h} (as appropriate) does not represent H, or, for compounds of formula I in which Y represents —P(O)(OR^{9d})₂ or S(O)₃R^{9c}, in which R^{9c} and R^{9d} represent H, a corresponding compound of formula I in which Y represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate);
(viii) for compounds of formula I in which Y¹ and/or, if present, Y^{1a} represents —C(O)OR^{9b}, S(O)₃R^{9c}, —P(O)(OR^{9d})₂, —P(O)(OR^{9e})N(R^{10f})R^{9f} or —B(OR^{9h})₂ and R^{9b} to R^{9e} and R^{9h} do not represent H:
(A) esterification (or the like) of a corresponding compound of formula I in which R^{9b} to R^{9e} and R^{9h} represent H; or
(B) trans-esterification (or the like) of a corresponding compound of formula I in which R^{9b} to R^{9e} and R^{9h} do not represent H (and does not represent the same value of the corresponding R^{9b} to R^{9e} and R^{9h} group in the compound of formula I to be prepared),
in the presence of the appropriate alcohol of formula XII,
R^{9za}OH XII
in which R^{9za} represents R^{9b} to R^{9e} or R^{9h} (as appropriate) provided that it does not represent H;
(ix) for compounds of formula I in which Y¹ and/or, if present, Y^{1a} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L¹ and/or, if present, L^{1a}, are as defined in claim 1, provided that they do not represent —(CH₂)ₚ-Q-(CH₂)_{q}— in which p represents 0 and Q represents —O—, reaction of a compound of formula XIII, 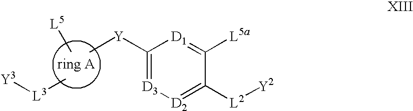
wherein at least one of L⁵ and L^{5a} represents an appropriate alkali metal group, a —Mg-halide, a zinc-based group or a suitable leaving group, or a protected derivative thereof, and the other may represent -L¹-Y¹ or -L^{1a}-Y^{1a} (as appropriate), and Y, ring A, D₁, D₂, D₃, L², Y², L³ and Y³ are as defined in claim 1, with a compound of formula XIV,
L⁶-L^{xy}-Y^{b} XIV
wherein L^{xy} represents L¹ or L^{1a} (as appropriate) and Y^{b} represents —C(O)OR^{9b}, —S(O)₃R^{9c}, —P(O)(OR⁹)₂, —P(O)(OR^{9e})N(R¹⁰)R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L⁶ represents a suitable leaving group;
(x) compounds of formula I in which L¹ and/or, if present, L^{1a} represent a single bond, and Y¹ and/or, if present, Y^{1a} represent either: B(OR^{9h})₂ in which R^{9h} represents H; —S(O)₃R^{9c}; or any one of the following groups: 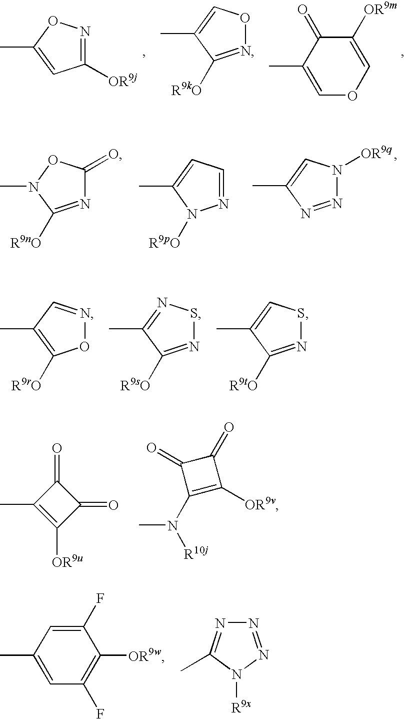
in which
R^{9j}, R^{9k}, R^{9m}, R⁹ⁿ, R^{9p}, R^{9r}, R^{9s}, R^{9t}, R^{9u}, R^{9v}, R^{10j} and R^{9x} represent hydrogen, and R^{9w} is as defined in claim 1, may be prepared in accordance with the procedures described in international patent application WO 2006/077366;
(xi) compounds of formula I in which L¹ and/or, if present, L^{1a} represent a single bond, and Y¹ and/or, if present, Y^{1a} represent any one of the following groups: 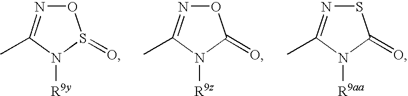
in which R^{9y}, R^{9z} and R^{9aa} represent H, reaction of a compound corresponding to a compound of formula I, but in which Y¹ and/or, if present, Y^{1a} represents —CN, with hydroxylamine (so forming a corresponding hydroxyamidino compound) and then with SOCl₂, R^{j}—OC(O)Cl (wherein R^{j} represents a C₁₋₆ alkyl group) or thiocarbonyl diimidazole, respectively;
(xii) compounds of formula I in which L¹ and/or, if present, L^{1a} represent a single bond, and Y¹ and/or, if present, Y^{1a} represent any one of the following groups: 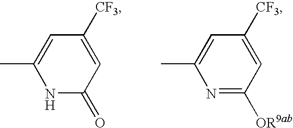
in which R^{9ab} is as defined in claim 1, may be prepared by reaction of a compound of formula XIII wherein at least one of L⁵ and L^{5a} represents an appropriate alkali metal group, a —Mg-halide, a zinc-based group or a suitable leaving group, and the other may represent -L¹-Y¹ or -L^{1a}-Y^{1a} (as appropriate), and ring A, D₁, D₂ₐ, D_{2b}, D₃, L³ and Y³ are as defined in claim 1, with a compound of formula XIVa or XIVb, 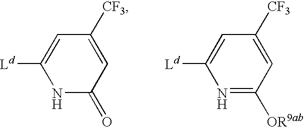
wherein R^{ab} is as defined in claim 1 and L^{d} represents (as appropriate) an appropriate alkali metal group, a —Mg-halide, a zinc-based group or a suitable leaving group;
(xiii) for compounds of formula I in which L¹ and/or, if present, L^{1a} represent a single bond, and Y¹ and/or, if present, Y^{1a} represent —C(O)OR^{9b} in which R^{9b} is H, reaction of a compound of formula XIII as defined above but in which L⁵ and/or L^{5a} (as appropriate) represents either:
(I) an alkali metal; or
(II) —Mg-halide,
with carbon dioxide, followed by acidification;
(xiv) for compounds of formula I in which L¹ and/or, if present, L^{1a} represent a single bond, and Y¹ and/or, if present, Y^{1a} represent —C(O)OR^{9b}, reaction of a corresponding compound of formula XIII as defined above but in which L⁵ and/or L^{5a} (as appropriate) is a suitable leaving group with CO (or a reagent that is a suitable source of CO), in the presence of a compound of formula XV,
R^{9b}OH XV
wherein R^{9b} is as defined in claim 1, and an appropriate catalyst system;
(xv) for compounds of formula I in which Y represents —C(O)—, reaction of either a compound of formula XVI or XVII, 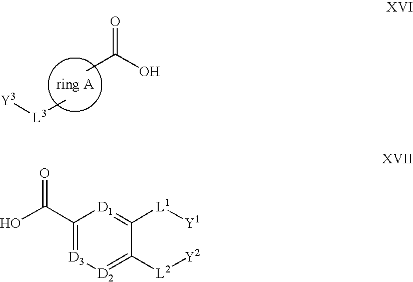
respectively with a compound of formula XVIII or XIX, 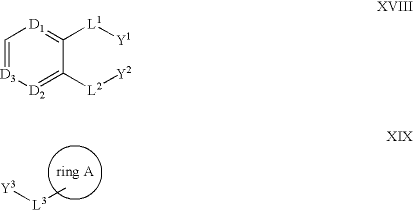
wherein (in all cases) ring A, D₁, D₂, D₃, L¹, Y¹, L², Y², L³ and Y³ are as defined in claim 1;
(xvi) for compounds of formula I in which Y represents —C(O)—, reaction of either a compound of formula XX or XXI, 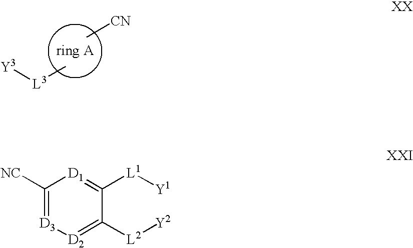
respectively with a compound of formula XXII or XXIII, 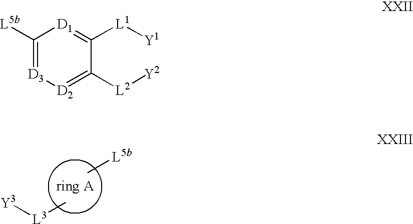
wherein L^{5b} represents L⁵ as defined above provided that it does not represent -L¹-Y¹, and (in all cases) ring A, D₁, D₂, D₃, L¹, Y¹, L², Y², L³ and Y³ are as defined in claim 1;
(xvii) for compounds of formula I in which Y represents —C(O)—, reaction of an activated derivative of a compound of formula XVI or XVII as defined above, with a compound of formula XXII or XXIII (as defined above), respectively;
(xviii) for compounds of formula I in which Y represents —C(═N—OR²⁸)—, reaction of a corresponding compound of formula I, with a compound of formula XXIIIA,
H₂N—O—R²⁸ XXIIIA
wherein R²⁸ is as defined in claim 1;
(xix) for compounds of formula I in which Y represents —C(═NR²⁸)— and R²⁸ represents C₁₋₆ alkyl optionally substituted by one or more halo atoms, reaction of a corresponding compound of formula I, in which R²⁸ represents hydrogen, with a compound of formula XXIIIB,
R^{28a}-L⁷ XXIIIB
wherein R^{28a} represents R²⁸, provided that it does not represent hydrogen and L⁷ represents a suitable leaving group;
(xx) compounds of formula I in which -L¹-Y¹ and/or, if present, -L^{1a}-Y^{1a} represent —S(O)₃H, sulfonylation of a compound corresponding to a compound of formula I, but in which -L¹-Y¹ and/or -L^{1a}-Y^{1a} (as appropriate) represents hydrogen;
(xxi) compounds of formula I in which -L¹-Y¹ and/or, if present, -L^{1a}-Y^{1a} represent —S(O)₃H, oxidation of a compound corresponding to a compound of formula I, but in which -L¹-Y¹ and/or -L^{1a}-Y^{1a} (as appropriate) represents —SH.

73. A process for the preparation of a pharmaceutical formulation as defined in claim 62, which process comprises bringing into association a compound as defined in claim 1, but without proviso (B), or a pharmaceutically acceptable salt thereof with a pharmaceutically-acceptable adjuvant, diluent or carrier.

74. A process for the preparation of a combination product as defined in claim 69, which process comprises bringing into association a compound as defined in claim 1, but without the provisos, or a pharmaceutically acceptable salt thereof with the other therapeutic agent that is useful in the treatment of a respiratory disorder and/or inflammation, and at least one pharmaceutically-acceptable adjuvant, diluent or carrier.
